(19) United States
(12) Patent Application Publication
(10) Pub. No.: US 2021/0024649 A1
(43) Pub. Date: Jan. 28, 2021

(54) Methods and Compositions for Antibody and Antibody-loaded Dendritic Cell Mediated Therapy

(71) Applicant: The Board of Trustees of the Leland Stanford Junior University, Stanford, CA (US)

(72) Inventors: Edgar George Engleman, Atherton, CA (US); Yaron Carmi, Palo Alto, CA (US)

(21) Appl. No.: 16/998,734

(22) Filed: Aug. 20, 2020

Related U.S. Application Data
(62) Division of application No. 15/112,409, filed on Jul. 18, 2016.
(60) Provisional application No. 62/066,574, filed on Oct. 21, 2014.
(60) Provisional application No. 61/930,386, filed on Jan. 22, 2014.

(51) Int. Cl. C07K 16/30; A61K 38/17; A61K 38/19; A61K 39/395; A61K 45/06; C07K 16/06; A61K 31/713; A61K 31/7105; A61K 31/4745; A61K 31/711; A61K 35/15; A61K 38/21; A61K 39/00

(52) U.S. Cl. CPC C07K 16/30; C07K 2317/92; A61K 38/191; A61K 39/39558; A61K 45/06; C07K 16/06; A61K 31/713; A61K 31/7105; A61K 31/4745; A61K 31/711; A61K 38/19; A61K 35/15; A61K 38/217; A61K 39/0011; A61K 2039/505; C07K 2317/73; A61K 2039/5154; A61K 38/177

(57) ABSTRACT

Methods, compositions, and kits are provided for inducing an immune response in an individual (e.g., an individual having cancer). Aspects of the methods include administering an antibody composition having an allogeneic IgG antibody; and administering a treatment that activates antigen presenting cells. In some cases, the antibody composition includes polyclonal allogeneic IgG antibodies with a plurality of binding specificities. In some cases, the polyclonal antibodies are from sera pooled from 2 or more individuals. In some cases, the methods include administering an antigen presenting cell stimulatory agent. Aspects of the methods also include contacting an antigen presenting cell (dendritic cell (DC)) from an individual with a target antigen and an antibody composition having an allogeneic IgG antibody to produce a loaded APC, which can be used to induce an immune response in the individual. Aspects of the methods also include contacting a T cell of the individual with the loaded APC.

### CROSS-REFERENCE

This application is a divisional of U.S. patent application Ser. No. 15/112,409, filed Jul. 18, 2016, which is a national stage entry of International Patent Application No. PCT/US2015/012511, filed Jan. 22, 2015 which claims the benefit of U.S. Provisional Patent Application No. 62/066,574, filed Oct. 21, 2014 and U.S. Provisional Patent Application No. 61/930,386, filed Jan. 22, 2014, each of which applications is incorporated herein by reference in its entirety.

### GOVERNMENT RIGHTS

This invention was made with Government support under contract CA141468 awarded by the National Institutes of Health. The Government has certain rights in the invention.

### BACKGROUND

Despite the ability of the immune system to distinguish subtle differences between self and non-self, cancers tend to grow and spread, often leading to the death of their hosts. An adaptive T cell response to tumor associated antigens (TAA) can occur in this setting, resulting in tumor regression or cessation of tumor growth for variable periods. However, most tumors eventually escape via immunoediting, the process whereby tumor cells evade immune detection through selection of variants that do not appropriately express the antigens recognized by effector T cells.

In contrast to autologous tumors, allogeneic tumors derived from genetically distinct individuals or mouse strains are reliably rejected when transferred to immunologically intact hosts, much like transplanted allogeneic organs. Remarkably, this occurs even when the tumor and host share the same alleles of the major histocompatibility complex (MHC) antigens, which have long been thought to be the primary determinants of transplant rejection.

Under these conditions, a variety of minor histocompatibility antigens are processed and presented in association with MHC class I or II molecules, resulting in the generation of effector T cells that attack tumors in an antigen specific manner. Such antigens are often comprised of polymorphic sequences of common proteins but can also result from gene deletions, differences in intracellular processing of peptides and other intracellular mechanisms. Presumably, due to the number and variety of unique proteins expressed by allogeneic tumors, these tumors do not escape the host T cell response. Indeed, recognition of minor histocompatibility antigens by T cells derived from the donor is believed to be the main reason why allogeneic hematopoietic cell transplants can cure certain cancers.

Regardless of tumor type or setting, antigen presenting cells (APCs) are thought to be responsible for processing and presenting TAA to T cells. Among APCs, classically activated dendritic cells (DC) and macrophages can give rise to a T cell-mediated immune response, which mediates tumor cytotoxicity and regression. Loading and activation of DC with TAA ex vivo can induce a clinically significant anti-tumor immune response in advanced cancer patients. Nonetheless, tumor associated DC generally fail to induce an effective response in the autologous setting and may even suppress anti-tumor immunity.

Given the wide range of mechanisms enabling tumors to evade immune mediated destruction, the mechanisms by which APCs generate an effective immune response against allogeneic tumors remains unexplained, yet these processes have important clinical implications. There is need in the art for compositions and methods for inducing effective anti-tumor immune responses. Identifying the mechanism responsible for inducing effective anti-tumor immunity in the allogeneic setting has enabled the discovery and development of novel and effective methods to treat autologous tumors.

### Publications

Steinman et al., Nature. 2007 Sep. 27; 449(7161):419-26; Kurts et al., Nat Rev Immunol. 2010 June; 10(6):403-14; Trombetta et. al., Annu Rev Immunol. 2005; 23:975-1028; Fong et al., J Immunol. 2001 Mar. 15; 166(6):4254-9; Hsu et. al., Nat Med. 1996 January; 2(1):52-8; Fong et. al., Annu Rev Immunol. 2000; 18:245-73; Gilboa et al, J Clin Invest. 2007 May; 117(5):1195-203; Melief et. al., Immunity. 2008 Sep. 19; 29(3):372-83; Palucka et al., Immunity. 2013 Jul. 25; 39(1):38-48; Tseng et al., Proc Natl Acad Sci USA. 213 July 2110(27):11103-8; Schuurhuis et al., J Immunol. 2006 Apr. 15; 176(8):4573-80; U.S. patent application number US20020155108; and U.S. Pat. No. 8,518,405.

### SUMMARY

Methods are provided for treating an individual having cancer. Aspects of the methods include administering to the individual: (i) an antibody composition having an allogeneic IgG antibody that specifically binds to an antigen of a cancer cell of the individual; and (ii) a treatment that activates dendritic cells of the individual. In some cases, the antibody composition includes polyclonal allogeneic IgG antibodies with a plurality of binding specificities. In some cases, the polyclonal allogeneic IgG antibodies can be a group of monoclonal antibodies (e.g., with defined target antigen specificities). In some cases, the polyclonal allogeneic IgG antibodies can be from sera from one or more individuals (e.g., sera from one individual, sera pooled from two or more individuals, etc.). In some cases, the antibody composition includes intravenous immunoglobulin (IVIG) or antibodies purified or enriched from IVIG. In some cases, the treatment that activates dendritic cells of the individual includes exposing the individual to local irradiation. In some cases, the treatment that activates dendritic cells of the individual includes administering to the individual a stimulatory composition having a dendritic cell stimulatory agent. In some cases, a dendritic cell stimulatory agent is conjugated to an allogeneic IgG antibody. In some cases, the stimulatory composition includes a CD40 agonist and a proinflammatory cytokine (e.g, TNFα, IFNγ, etc.). In some cases, the stimulatory composition includes a Toll like receptor (TLR) agonist.

Methods are provided for inducing an immune response in an individual. Aspects of the methods include: (a) contacting in vitro a dendritic cell (DC) from the individual (e.g. a population of dendritic cells from the individual) with a target antigen and an antibody composition having an allogeneic IgG antibody that specifically binds to the target antigen, at a dose and for a period of time effective for the uptake of the target antigen by the DC, thereby producing a loaded DC (e.g. a population of loaded DC); and (b) contacting a T cell of the individual (e.g., a population of T cells of the individual) with the loaded DC, where the loaded DC presents antigens to the T cell to produce a contacted T cell, and the contacted T cell generates an immune response specific to the presented antigens. In some cases, the DC is from an individual with cancer and the target antigen is associated with the cancer. In some cases, the DC is contacted with a cancer cell from the individual. In some cases, the DC is contacted with a lysate from cancer cells of the individual. In some cases, the DC is contacted with one or more (e.g., two or more) plasma membrane proteins from cancer cells of the individual. In some cases, the DC is contacted with a stimulatory composition comprising a DC stimulatory agent. In some cases, the stimulatory composition comprises a CD40 agonist and a proinflammatory cytokine. In some cases, the stimulatory composition comprises a TLR agonist. In some cases, the DC stimulatory agent is conjugated to an allogeneic IgG antibody. In some cases, the target antigen (e.g., a target cell, a cancer cell lysate/extract, a composition having two or more plasma membrane proteins) is contacted with the antibody composition, producing an immune complex, prior to contacting the DC. Thus, in some cases, the methods include contacting a DC with an immune complex. In some cases, the DC is simultaneously contacted with the target antigen and the antibody composition. In some cases, the step of contacting a T cell is performed in vivo and the method comprises introducing the loaded DC into the individual. In some cases, the step of contacting a T cell is performed in vitro and the method comprises introducing the contacted T cell into the individual.

Compositions and kits for practicing the methods of the disclosure are also provided. In some cases, a subject composition includes: polyclonal allogeneic IgG antibodies with a plurality of binding specificities; and at least one DC stimulatory agent. In some cases, a subject composition includes: polyclonal allogeneic IgG antibodies with a plurality of binding specificities; a CD40 agonist; and a proinflammatory cytokine (e.g., TNFα, IL-1α, IL-10, IL-19, interferon gamma (IFNγ), and the like). In some cases, a DC stimulatory agent is conjugated to at least one of the allogeneic IgG antibodies of the composition. In some cases, a subject composition includes intravenous immunoglobulin (IVIG) or antibodies purified or enriched from IVIG. In some cases, a subject composition includes IVIG or antibodies purified or enriched from IVIG, where at least one of the allogeneic IgG antibodies present in the composition is conjugated to a DC stimulatory agent. In some cases, at least one of the allogeneic IgG antibodies present in the composition is conjugated to a CD40 agonist and at least one of the allogeneic IgG antibodies present in the composition is conjugated to a proinflammatory cytokine. In some cases, at least one of the allogeneic IgG antibodies present in the composition is conjugated to a CD40 agonist; at least one of the allogeneic IgG antibodies present in the composition is conjugated to a proinflammatory cytokine; or at least one of the allogeneic IgG antibodies present in the composition is conjugated to a CpG oligodeoxynucleotide (CpG ODN).

In some embodiments, a composition comprising an allogenic IgG antibody and an APC stimulating composition for use in reducing the size of a tumor is provided.

In one aspect, the present invention provides a method of treating an individual having cancer, the method comprising: administering to the individual: (i) an antibody composition comprising an allogeneic IgG antibody that binds to an antigen of a cancer cell of the individual; and (ii) a treatment that activates an APC of the individual, wherein the APC is a dendritic cell, a macrophage, or a B-cell, thereby treating an individual having cancer.

In some embodiments, the allogeneic IgG antibody binds the antigen on the cancer cell in the individual to form an immunocomplex. In some cases, the activation of the APC comprises uptake of the immunocomplex by the APC and presentation of multiple antigens of the cancer cell to T cells in the individual. In some cases, at least one of the multiple antigens presented to T cells is different than the antigen in the immunocomplex.

In some cases, the method reduces the number of cancer cells, or the size of one or more tumors, in the individual. In some cases, the cancer is a solid tumor. In some cases, the solid tumor is less than 1 cm in diameter. In some cases, the individual is a human.

In some cases, the allogeneic IgG antibody binds an antigen that is present in at least 10,00 copies on the surface of the cancer cell. In some cases, the allogeneic IgG antibody binds the antigen on the cancer cell at an affinity at least 100, 1000, 10000× higher (Kd 100, 1000, 10000× lower) than an antigen on a non-cancer cell, wherein the antigen on the cancer cell has one or more polymorphisms as compared to the antigen on the non-cancer cell. In some cases, the allogeneic IgG antibody binds the cancer cell with higher avidity than the allogeneic IgG antibody binds a non-cancer cell.

In some embodiments, the treatment that activates a dendritic cell comprises a dendritic cell stimulatory composition comprising a dendritic cell stimulatory agent. In some cases, the dendritic cell stimulatory composition comprises one or more dendritic cell stimulatory agents selected from the group consisting of: (i) a Toll-like receptor (TLR) agonist; (ii) a CD40 agonist; (iii) a CD40 agonist and a proinflammatory cytokine; (iv) a checkpoint molecule neutralizing compound; (v) an indoleamine 2,3-dioxygenase (IDO) inhibitor; (vi) an NFkB activator; (vii) a compound that opens calcium channels; and (viii) a T cell-related co-stimulatory molecule. In some cases, the dendritic cell stimulatory composition comprises a CD40 agonist and a proinflammatory cytokine. In some cases, the proinflammatory cytokine is tumor necrosis factor alpha (TNFα) and/or IFNγ. In some cases, the dendritic cell stimulatory agent is conjugated to an allogeneic IgG antibody.

In some embodiments, the treatment that activates a B-cell comprises a B-cell stimulatory composition containing a B-cell stimulatory agent. In some cases, the B-cell stimulatory composition comprises one or more B-cell stimulatory agents selected from the group consisting of: (i) a Toll-like receptor (TLR) agonist; (ii) a CD40 agonist; (iii) a CD40 agonist and a proinflammatory cytokine; (iv) an antigen that binds the B-cell receptor (v) an anti-idiotype antibody; (vi) and an agent that cross-links surface immunoglobulin. In some cases, the proinflammatory cytokine is IL-1, IL-2, IL-3, IL-4, IL-6, IL-7, IL-9, IL-10, IL-12, IL-15, IL-18, IL-21, IFN-α, IFN-β, IFN-γ, G-CSF, or GM-CSF. In some cases, the TLR agonist is CpG ODN, immunostimulatory DNA, immunostimulatory RNA, immunostimulatory oligonucleotides, Imiquimod, Resiquimod, Loxribine, Flagellin, FSL-I or LPS. In some cases, the antigen is a self antigen, an allogeneic antigen, a peptide antigen, a nucleic acid antigen, a carbohydrate antigen, or a tumor associated antigen. In some cases, the agent that cross-links surface immunoglobulin is an anti-Ig antibody, an anti-idiotype antibody, or an anti-isotype antibody. In some cases, the B-cell stimulatory agent is conjugated to an allogeneic IgG antibody.

In some embodiments, the treatment that activates a macrophage comprises a macrophage stimulatory composition containing a macrophage stimulatory agent. In some cases, the macrophage stimulatory composition comprises one or more macrophage stimulatory stimulatory agents selected from the group consisting of: (i) a Toll-like receptor (TLR) agonist; (ii) a macrophage activating cytokine; and (iii) a glucocorticoid receptor agonist. In some cases, the macrophage activating cytokine is IL-1, IL-4, IL-6, IL-10; IL-13, TNF-α, TNF-β, G-CSF, GM-CSF, or IFN-γ. In some cases, the TLR agonist is a TLR4 agonist or a TLR2 agonist. In some cases, the TLR4 or TLR2 agonist is lipopolysaccharide, muramyl dipeptide, lipoteichoic acid, or a bacterial heat shock protein. In some cases, the macrophage stimulatory agent is conjugated to an allogeneic IgG antibody. In some cases,

In some embodiments, the antigen of the cancer cell is an antigen that is enriched in cancer cells. In some embodiments, the allogeneic IgG antibody is a monoclonal antibody. In some embodiments, the antibody composition comprises two or more allogeneic IgG antibodies, wherein at least two of the two more allogeneic IgG antibodies specifically bind to different antigens. In some embodiments, the antibody composition comprises two or more allogeneic IgG antibodies, wherein at least two of the two more allogeneic IgG antibodies specifically bind to a different epitope of the same antigen. In some cases, at least two of the two more allogeneic IgG antibodies are monoclonal antibodies.

In some embodiments, at least one of: (a) said antibody composition; and (b) said treatment that activates an APC of the individual, is administered by local injection into or near (i) a tumor and/or (ii) a site of tumor resection. In some embodiments, at least one of (a) said antibody composition; and (b) said treatment that activates an APC of the individual, is administered in a liposome, a microparticle, or a nanoparticle.

In some embodiments, the APC is a dendritic cell. In some embodiments, the APC is a macrophage. In some embodiments, the APC is a B-cell.

In another aspect, the present invention provides a method of treating an individual having cancer, the method comprising: administering to the individual: (i) an antibody composition that comprises polyclonal allogeneic IgG antibodies that bind a plurality of antigens on a cancer cell; and (ii) a treatment that activates an antigen presenting cell (APC) of the individual, wherein the APC is a dendritic cell, a macrophage, or a B-cell. In some embodiments, the polyclonal allogeneic IgG antibodies are from serum from a second individual. In some embodiments, the polyclonal allogeneic IgG antibodies are pooled from 2 or more individuals.

In some embodiments, the target antigen of at least one of the allogeneic IgG antibodies is not predetermined. In some embodiments, the treatment that activates dendritic cells comprises a dendritic cell stimulatory composition comprising a dendritic cell stimulatory agent.

In some cases, the dendritic cell stimulatory composition comprises one or more dendritic cell stimulatory agents selected from: (i) a Toll-like receptor (TLR) agonist; (ii) a CD40 agonist; (iii) a CD40 agonist and a proinflammatory cytokine; (iv) a checkpoint molecule neutralizing compound; (v) an indoleamine 2,3-dioxygenase (IDO) inhibitor; (vi) an NFkB activator; (vii) a compound that opens calcium channels; and (viii) a T cell-related co-stimulatory molecule. In some cases, the dendritic cell stimulatory composition comprises a CD40 agonist and a proinflammatory cytokine. In some cases, the proinflammatory cytokine is tumor necrosis factor alpha (TNFα) and/or IFNγ. In some cases, the dendritic cell stimulatory agent is conjugated to at least one of the allogeneic IgG antibodies.

In some embodiments, the treatment that activates a B-cell comprises a B-cell stimulatory composition containing a B-cell stimulatory agent. In some cases, the B-cell stimulatory composition comprises one or more B-cell stimulatory agents selected from the group consisting of: (i) a Toll-like receptor (TLR) agonist; (ii) a CD40 agonist; (iii) a CD40 agonist and a proinflammatory cytokine; (iv) an antigen that binds the B-cell receptor; (v) an anti-idiotype antibody; (vi) and an agent that cross-links surface immunoglobulin. In some cases, the proinflammatory cytokine is IL-1, IL-2, IL-3, IL-4, IL-6, IL-7, IL-9, IL-10, IL-12, IL-15, IL-18, IL-21, IFN-α, IFN-β, IFN-γ, G-CSF, or GM-CSF. In some cases, the TLR agonist is CpG ODN, immunostimulatory DNA, immunostimulatory RNA, immunostimulatory oligonucleotides, Imiquimod, Resiquimod, Loxribine, Flagellin, FSL-I or LPS. In some cases, the antigen is a self antigen, an allogeneic antigen, a peptide antigen, a nucleic acid antigen, a carbohydrate antigen, or a tumor associated antigen. In some cases, the agent that cross-links surface immunoglobulin is an anti-Ig antibody, an anti-idiotype antibody, or an anti-isotype antibody. In some cases, the B-cell stimulatory agent is conjugated to an allogeneic IgG antibody.

In some embodiments, the treatment that activates a macrophage comprises a macrophage stimulatory composition containing a macrophage stimulatory agent. In some cases, the macrophage stimulatory composition comprises one or more macrophage stimulatory stimulatory agents selected from the group consisting of: (i) a Toll-like receptor (TLR) agonist; (ii) a macrophage activating cytokine; and (iii) a glucocorticoid receptor agonist. In some cases, the macrophage activating cytokine is IL-1, IL-4, IL-6, IL-10; IL-13, TNF-α, TNF-β, G-CSF, GM-CSF, or IFN-γ. In some cases, the TLR agonist is a TLR4 agonist or a TLR2 agonist. In some cases, the TLR4 or TLR2 agonist is lipopolysaccharide, muramyl dipeptide, lipoteichoic acid, or a bacterial heat shock protein. In some cases, the macrophage stimulatory agent is conjugated to an allogeneic IgG antibody.

In some embodiments, at least one of (a) said antibody composition; and (b) said treatment that activates an APC of the individual, is administered by local injection into or near (i) a tumor and/or (ii) a site of tumor resection. In some embodiments, at least one of: (a) said antibody composition; and (b) said treatment that activates an APC of the individual, is administered in a liposome, a microparticle, or a nanoparticle.

In some embodiments, the polyclonal allogeneic IgG antibodies are two or more monoclonal antibodies. In some cases, at least two of the two or more monoclonal antibodies specifically bind an antigen that is enriched in cancer cells. In some cases, at least two of the two more monoclonal antibodies specifically bind to different antigens. In some cases, at least two of the two or more monoclonal antibodies specifically bind to two different epitopes on the same antigen.

In some embodiments, the polyclonal allogeneic IgG antibodies bind antigens on the cancer cell in the individual to form an immunocomplex. In some cases, the activation of the APC comprises uptake of the immunocomplex by the APC and presentation of multiple antigens of the cancer cell to T cells in the individual. In some cases, at least one of the multiple antigens presented to T-cells is different from any of the antigens in the immunocomplex.

In some embodiments, the method reduces the number of cancer cells, or reduces the size of a tumor, in the individual. In some cases, the cancer is a solid tumor. In some cases, the solid tumor is less than 1 cm in diameter. In some cases, the individual is human.

In another aspect, the present invention provides a method of inducing an immune response in an individual, the method comprising: (a) contacting in vitro an antigen presenting cell (APC) from the individual with: (i) a cancer cell or portion thereof; and (ii) an antibody composition comprising an allogeneic IgG antibody that binds to an antigen on the cancer cell, wherein the cancer cell and allogeneic IgG antibody that binds to the antigen on the cancer cell form an immunocomplex, and wherein said contacting results in the uptake of the immunocomplex by the APC, thereby producing a loaded APC, wherein the APC is a dendritic cell, a macrophage, or a B-cell; and (b) contacting a T cell of the individual with the loaded APC, wherein the loaded APC presents cancer cell antigens to the T cell to produce a contacted T cell, and the contacted T cell generates an immune response specific to the presented cancer cell antigens.

In some embodiments, the APC is a dendritic cell selected from the group consisting of: a bone marrow derived DC, a blood derived DC, a splenic DC, and a tumor associated DC (TADC). In some embodiments, the method further comprises contacting the APC with an APC stimulatory composition comprising an APC stimulatory agent. In some cases, the APC stimulatory composition is a dendritic cell stimulatory composition comprising a dendritic cell stimulatory agent.

In some cases, the dendritic cell stimulatory composition comprises one or more dendritic cell stimulatory agents selected from: (i) a Toll-like receptor (TLR) agonist; (ii) a CD40 agonist; (iii) a CD40 agonist and a proinflammatory cytokine; (iv) a checkpoint molecule neutralizing compound; (v) an indoleamine 2,3-dioxygenase (IDO) inhibitor; (vi) an NFkB activator; (vii) a compound that opens calcium channels; and (viii) a T cell-related co-stimulatory molecule. In some cases, the dendritic cell stimulatory composition comprises a CD40 agonist and a proinflammatory cytokine. In some cases, the proinflammatory cytokine is tumor necrosis factor alpha (TNFα) and/or IFNγ. In some cases, the dendritic cell stimulatory agent is conjugated to the allogeneic IgG antibody.

In some embodiments, the APC stimulatory composition is a B-cell stimulatory composition comprising a B-cell stimulatory agent. In some cases, the B-cell stimulatory composition comprises one or more B-cell stimulatory agents selected from the group consisting of: (i) a Toll-like receptor (TLR) agonist; (ii) a CD40 agonist; (iii) a CD40 agonist and a proinflammatory cytokine; (iv) an antigen that binds the B-cell receptor; (v) an anti-idiotype antibody; (vi) and an agent that cross-links surface immunoglobulin. In some cases, the proinflammatory cytokine is IL-1, IL-2, IL-3, IL-4, IL-6, IL-7, IL-9, IL-10, IL-12, IL-15, IL-18, IL-21, IFN-α, IFN-β, IFN-γ, G-CSF, or GM-CSF. In some cases, the TLR agonist is CpG ODN, immunostimulatory DNA, immunostimulatory RNA, immunostimulatory oligonucleotides, Imiquimod, Resiquimod, Loxribine, Flagellin, FSL-I or LPS. In some cases, the antigen is a self antigen, an allogeneic antigen, a peptide antigen, a nucleic acid antigen, a carbohydrate antigen, or a tumor associated antigen. In some cases, the agent that cross-links surface immunoglobulin is an anti-Ig antibody, an anti-idiotype antibody, or an anti-isotype antibody. In some cases, the B-cell stimulatory agent is conjugated to an allogeneic IgG antibody.

In some embodiments, the APC stimulatory composition is a macrophage stimulatory composition comprising a macrophage stimulatory agent. In some cases, the macrophage stimulatory composition comprises one or more macrophage stimulatory agents selected from the group consisting of (i) a Toll-like receptor (TLR) agonist; (ii) a macrophage activating cytokine; and (iii) a glucocorticoid receptor agonist. In some cases, the macrophage activating cytokine is IL-1, IL-4, IL-6, IL-10; IL-13, TNF-α, TNF-β, G-CSF, GM-CSF, or IFN-γ. In some cases, the TLR agonist is a TLR4 agonist or a TLR2 agonist. In some cases, the TLR4 or TLR2 agonist is lipopolysaccharide, muramyl dipeptide, lipoteichoic acid, or a bacterial heat shock protein. In some cases, the macrophage stimulatory agent is conjugated to an allogeneic IgG antibody.

In some embodiments, the cancer cell is contacted with the antibody composition prior to contacting the APC. In some embodiments, the APC is simultaneously contacted with the cancer cell and the antibody composition. In some embodiments, the step of contacting a T cell is performed in vivo and the method comprises introducing the loaded APC into the individual. In some embodiments, the step of contacting a T cell is performed in vitro and the method comprises introducing the contacted T cell into the individual. In some embodiments, the allogeneic IgG antibody is a monoclonal antibody. In some embodiments, the antibody composition comprises polyclonal allogeneic IgG antibodies that bind a plurality of cancer cell antigens. In some cases, the polyclonal allogeneic IgG antibodies are two or more monoclonal antibodies.

In another aspect, the present invention provides a composition for loading APCs, the composition comprising: (i) an antibody composition comprising an allogeneic IgG antibody that binds to an antigen of a cancer cell; and (ii) an APC stimulatory agent, wherein the APC stimulatory agent is a dendritic cell stimulatory agent, a macrophage stimulatory agent, or a B-cell stimulatory agent. In some embodiments, the allogeneic IgG antibody is a monoclonal antibody.

In some embodiments, the antibody composition comprises polyclonal allogeneic IgG antibodies that bind a plurality of cancer cell antigens. In some cases, the polyclonal allogeneic IgG antibodies comprises two or more monoclonal antibodies. In some cases, at least two of the two or more monoclonal antibodies specifically bind an antigen that is enriched in cancer cells. In some cases, at least two of the two or more monoclonal antibodies specifically bind to different antigens. In some cases, at least two of the two or more monoclonal antibodies specifically bind to a different epitope of the same antigen.

In some cases, the polyclonal allogeneic IgG antibodies are from serum from an individual. In some cases, the polyclonal allogeneic IgG antibodies are pooled from 2 or more individuals. In some cases, the composition comprises intravenous immunoglobulin (IVIG) or antibodies purified or enriched from IVIG.

In some embodiments, the dendritic cell stimulatory agent is selected from the group consisting of: (i) a Toll-like receptor (TLR) agonist; (ii) a CD40 agonist; (iii) a CD40 agonist and a proinflammatory cytokine; (iv) a checkpoint molecule neutralizing compound; (v) an indoleamine 2,3-dioxygenase (IDO) inhibitor (vi) an NFkB activator; (vii) a compound that opens calcium channels; and (viii) a T cell-related co-stimulatory molecule.

In some embodiments, the B-cell stimulatory agent is selected from the group consisting of: (i) a Toll-like receptor (TLR) agonist; (ii) a CD40 agonist; (iii) a CD40 agonist and a proinflammatory cytokine; (iv) an antigen that binds the B-cell receptor; (v) an anti-idiotype antibody; (vi) and an agent that cross-links surface immunoglobulin.

In some embodiments, the macrophage stimulatory agent is selected from the group consisting of: (i) a Toll-like receptor (TLR) agonist; (ii) a macrophage activating cytokine; and (iii) a glucocorticoid receptor agonist.

In some embodiments, at least one allogeneic IgG antibody of the antibody composition is conjugated to the APC stimulatory agent. In some cases, at least one allogeneic IgG antibody of the antibody composition is conjugated to a CD40 agonist, and at least one allogeneic IgG antibody of the antibody composition is conjugated to a proinflammatory cytokine. In some cases, the proinflammatory cytokine is TNFα and/or IFNγ.

In some embodiments, at least one allogeneic IgG antibody of the antibody composition is conjugated to a CD40 agonist; at least one allogeneic IgG antibody of the antibody composition is conjugated to a proinflammatory cytokine; and at least one allogeneic IgG antibody of the antibody composition is conjugated to a Toll-like receptor (TLR) agonist.

In another aspect, the present invention provides a kit for use in any of the foregoing methods. In another aspect, the present invention provides a kit comprising: (i) a compartment comprising an antibody composition comprising an allogeneic IgG antibody that binds to an antigen of a cancer cell; and (ii) at least one compartment comprising at least one APC stimulatory composition, wherein the APC stimulatory composition is a dendritic cell stimulatory composition, a macrophage stimulatory composition, or a B-cell stimulatory composition.

In some embodiments, the APC stimulatory composition comprises one or more dendritic cell stimulatory agents selected from: (i) a Toll-like receptor (TLR) agonist; (ii) a CD40 agonist; (iii) a CD40 agonist and a proinflammatory cytokine; (iv) a checkpoint molecule neutralizing compound; (v) an indoleamine 2,3-dioxygenase (IDO) inhibitor; (vi) an NFkB activator; (vii) a compound that opens calcium channels; and (viii) a T cell-related co-stimulatory molecule. In some cases, the CD40 agonist is CD40L and the proinflammatory cytokine is TNFα and/or IFNg. In some cases, the CD40 agonist and proinflammatory cytokine are in the same compartment. In some cases, the CD40 agonist and proinflammatory cytokine are in separate compartments.

In some embodiments, the APC stimulatory composition comprises one or more macrophage stimulatory agents selected from the group consisting of: (i) a Toll-like receptor (TLR) agonist; (ii) a macrophage activating cytokine; and (iii) a glucocorticoid receptor agonist. In some embodiments, the APC stimulatory composition comprises one or more B-cell stimulatory agents selected from the group consisting of: (i) a Toll-like receptor (TLR) agonist; (ii) a CD40 agonist; (iii) a CD40 agonist and a proinflammatory cytokine; (iv) an antigen that binds the B-cell receptor; (v) an anti-idiotype antibody; (vi) and an agent that cross-links surface immunoglobulin.

In another aspect, the present invention provides a method for reducing the size or number of cells in a tumor, comprising: contacting the tumor with (i) an antibody composition comprising an allogeneic IgG antibody that specifically binds to an antigen of a tumor cell, and (ii) an APC stimulatory composition, wherein the APC is a dendritic cell, a macrophage, or a B-cell, thereby reducing the size of the tumor or number of cells in the tumor. In some embodiments, the contacting the tumor comprises simultaneous or sequential direct injection of the antibody composition and APC stimulatory composition into or near the site of the tumor. In some embodiments, the APC is a dendritic cell, and the APC stimulatory composition comprises a dendritic cell stimulatory agent. In some embodiments, the APC is a macrophage, and the APC stimulatory composition comprises a macrophage stimulatory agent. In some embodiments, the APC is a B-cell, and the APC stimulatory composition comprises a B-cell stimulatory agent.

In some cases, the APC stimulatory composition comprises one or more dendritic cell stimulatory agents selected from: (i) a Toll-like receptor (TLR) agonist; (ii) a CD40 agonist; (iii) a CD40 agonist and a proinflammatory cytokine; (iv) a checkpoint molecule neutralizing compound; (v) an indoleamine 2,3-dioxygenase (IDO) inhibitor (vi) an NFkB activator, (vii) a compound that opens calcium channels; and (viii) a T cell-related co-stimulatory molecule.

In some cases, the APC stimulatory composition comprises one or more macrophage stimulatory agents selected from the group consisting of: (i) a Toll-like receptor (TLR) agonist; (ii) a macrophage activating cytokine; and (iii) a glucocorticoid receptor agonist.

In some cases, the APC stimulatory composition comprises one or more B-cell stimulatory agents selected from the group consisting of (i) a Toll-like receptor (TLR) agonist; (ii) a CD40 agonist; (iii) a CD40 agonist and a proinflammatory cytokine; (iv) an antigen that binds the B-cell receptor; (v) an anti-idiotype antibody; (vi) and an agent that cross-links surface immunoglobulin.

### BRIEF DESCRIPTION OF THE DRAWINGS

The invention is best understood from the following detailed description when read in conjunction with the accompanying drawings. It is emphasized that, according to common practice, the various features of the drawings are not to-scale. On the contrary, the dimensions of the various features are arbitrarily expanded or reduced for clarity. Included in the drawings are the following figures.

FIG. 1a-1k. Tumor-binding antibodes initiate rejection of allogeneic tumors. a. Experimental design: Injection of LMP cells subcutaneously (s.c.) into 12951 syngeneic and C57Bl/6 allogeneic hosts. Injection of B16F10 cells s.c. into C57Bl/6 syngeneic and 1291 allogeneic hosts. b. Growth of LMP and B16 tumors in C57Bl/6 (▪), 129S1 (▴), CD4⁺ cell-depleted allogeneic mice (⋄) or CD8⁺ cell-depleted allogeneic mice (∘) (n=16). c. Percentages of LMP-infiltrating CD4⁺ and CD8⁺ T cells among CD45⁺ cells in 12951 (□) and C57Bl/6 mice (▪) (n=8). d. Percentages of LMP-infiltrating immature myeloid cells (iMC) and mature DC in 12951 (□) and C57Bl/6 mice (▪) (n=8). e. Myeloid cells in the draining lymph node of 1291 or C57Bl/6 mice inoculated with CFSE-labeled LMP cells 3 days earlier (n=6) f. Tumor uptake, MHCII and CD86 expression by syngeneic BMDC (□) and blood monocytes (Mo)-DC ( [symbol] ), and alloeneic BMDC (▪) and Mo-DC ( [symbol] ), incubated overnight with CFSE-labeled LMP cells (n=10). g. IgG and IgM bound in vivo to CFSE-labeled LMP cells 48 h after tumor inoculation into 12951 or C57Bl/6 mice. (n=5). h. and i. Staining of tumor sections for IgM (h) and IgG (i) 24 h following inoculation of CFSE-labeled LMP cells into 12951 and C57Bl/6 mice (n=5). j. Tumor growth in 129S1 (□), C57Bl/6 (▪) and B cell-depleted allogeneic hosts (♦). (n=6) k. Left: B16 tumor size in naive C57Bl/6 (∘), or in mice injected i.v. on days −1 and 0 with syngeneic-IgG (▪), syngeneic-IgM (▴), allogeneic-IgG (□), or allogeneic-IgM (Δ) (n=6). Right: B16 tumor size in naive C57Bl/6 (∘) injected twice with allogeneic-IgG (□) or allogeneic-IgM (▴), or FcγR KO mice (C57Bl/6 background) injected with allogeneic-IgG (▪) or allogeneic-IgM (▴) (n=6). Asterisk (*) denotes p<0.05 and two asterisks (**) denote p<0.01.

FIG. 2a-2h. AlloIgG-IC are taken up and presented by BMDC and drive protective immunity in vivo. a. Experimental design: Tumor lysates were incubated with syngeneic or allogeneic IgG or IgM and cultured with syngeneic BMDC overnight. b. Expression of CD86 and MHCII on DC cultured with antibodies and tumor lysates (□) or intact tumor cells (▪) (n=16). c. IL-12 and TNFα in the supematants of BMDC cultured overnight with LMP lysate (□) or intact LMP cells (▪) Ig-IC (n=16). d. BMDC incubated overnight with IC formed from CFSE-labeled tumor lysates (□) or CFSE-labeled intact cells (▪) (n=8). e. MHCII expression in BMDC cultured overnight with CFSE-labeled LMP cells and allogeneic antibodies (×400). f. Proliferation of CD4⁺ T cells cultured with DC loaded with IC formed from tumor lysates (□) or intact cells (▪) (n=8). g. Experimental design: tumors were removed from mice, coated with antibodies and incubated for 24 h with syngeneic DC. DC were washed and injected s.c. into corresponding tumor-resected mice. h. Effect on tumor recurrence of PBS (●), DC loaded with tumor lysate (∘), C57Bl/6 IgG-IC (▴), C57Bl/6 IgM-IC (Δ), 129S1 IgG-IC (▪) or 129S1 IgM-IC (□) (n=16).

FIG. 3a-3g. Tumor-associated dendritic cells (TADC), but not bone marrow-derived dendritic cells (BMDC), require stimulation in order to respond to allogG-IC. a. Tumor growth following intratumoral injection of PBS (∘), 129S1 IgG (⋄) or C57Bl/6 IgG (♦) (n=12). b. CD86 and MHCII expression on DC incubated with PBS (left bar for each condition), tumor lysates (middle bar for each condition) or alloIgG-IC (right bar for each condition) (n=9). c. TNFα and IL-12 in the supernatants of DC cultured alone (left bar for each condition), with LMP lysate (middle bar for each condition) or with allogG-IC (right bar for each condition) (n=12). d. Proliferation of CD4⁺ T cells cultured with DC (left bar for each condition), DC loaded with tumor lysate (middle bar for each condition) or DC loaded with alloIgG-IC (right bar for each condition) (n=12). e. Recurrence of resected LMP and B16 tumors in untreated mice (∘), or mice treated with allogG-IC activated BMDC (▪) or TADC (▴) (n=12). f. p-P38, pERK1/2 and pJNK in untreated DC (red), or DC incubated with alloIgG-IC. Graphs show arcsinh ratios of p-pP38, pERK1/2 and pJNK levels in DC incubated for 15 min with LMP lysate (left bar for each condition) or alloIgG-IC (right bar for each condition) (n=8). g. MHCII⁺ and CD86⁺ expression or CFSE levels of TADC after overnight culture with CFSE-labeled alloIgG-IC (n=12). PBS (left bar for each condition); IgG₂₉IC (right bar for each condition).

FIG. 4a-4i. Injection of tumors in situ with alloantibodies in combination with CD40L and TNFα induces systemic DC-mediated anti-tumor immunity. a. Tumor growth in mice untreated (∘), or injected with alloIgG (●), TNFα+CD40L (□), Polyl:C (Δ), allogG+TNFα+CD40L (▪) or Polyl:C+allogG (▴) (n=12). b. Mean fluorescence levels of PE in myeloid cells from B16-bearing mice 2 hours after injection of PBS (bottom), PE-labeled IgG (middle), or PE-labeled IgG with TNFα+CD40L (top). c. CD40 and CD86 expression on DC from B16 tumors 5 days following treatment (n=6). d. B16 growth in mice vaccinated with 2×10⁶ DC from B16 tumors untreated (∘), or injected with allogG (●), TNFα+CD40L (□), Poly I:C (Δ), TNFα+CD40L+alloIgG (▪) or Polyl:C+alloIgG (▴) (n=6). e. Tumor number in Tyr.CreER; Braf^{V800E}/Pten^{lox/lox} mice untreated (∘) or treated with alloIgG (□), TNFα+CD40L (Δ), or TNFα+CD40L+alloIgG (▪). Photographs show representative mice on the day of treatment and day 24 after treatment (n=8). f. 4T1 primary tumor size in untreated mice (∘), or mice treated with alloIgG (□), TNFα+CD40L (Δ), or TNFα+CD40L+alloIgG (♦) (n=7). g. Mean counts of visible lung metastases on day 30. Photographs and histology of lung metastases on day 30 (magnitude ×10, n=7). h. Antigen uptake and CD40/CD86 co-expression on TADC from lung cancer patients cultured overnight with CFSE-stained autologous tumor cells coated with selfIgG or alloIgG (n=2). i. DC HLA-DR upregulation and proliferative response of CD4⁺ T cells from mesothelioma (MSTO) patients after autologous BMDC culture with selfIgG- or alloIgG-coated autologous tumor cells (n=2).

FIG. 5a-5f. a. LMP (right) and B16 (left) growth in 1291 (□) C57Bl/6 (▪), or allogeneic hosts pretreated with anti-asialo-GM1 (Δ) or anti-NK1.1 antibodies (⋄). (n=5). b. BrdU uptake by CD4⁺ T cells (top graphs) and CD8⁺ T cells (bottom graphs) in lymphoid organs of 129S1 (□) and C57Bl/6 (▪) LMP-bearing mice c. Flow cytometric analysis of Gr-1^{neg}/CD11c⁺/MHCII⁺ cells from LMP-bearing mice (left panel) and B16-bearing mice (right panel). Histograms show representative expression levels of co-stimulatory molecules on DC from C57Bl/6 (blue) and 129S1 mice (red). d. IL-12 and TNFα in the supematants of syngeneic BMDC (□), syngeneic blood monocytes (Mo)-DC ( [symbol] ), allogeneic BMDC (▪) or Mo-DC ( [symbol] ) incubated with LMP cells overnight. e. Flow cytometric analysis of the binding of various concentrations of IgG from C57Bl/6 (□), IgM from C57Bl/6 (Δ), IgG from 1291 (▪) and IgM from 1291 (▴) to LMP and B16 cells. The lower panel shows a representative histogram of the MFI of IgG (left) or IgM (right) after incubation of 1 μg of C57Bl/6 (red) or 129S1 (green) antibodies with 1×10⁵ LMP (upper) or B16 (lower) cells. f. LMP tumor size in näive 129S1 mice injected with allogeneic IgG (▪), allogeneic IgM (▴), syngeneic IgG (□) or syngeneic IgM (Δ). (n=6). Asterisk (*) denotes p<0.05 and two asterisks (**) denote p<0.01.

FIG. 6a-6j. a. Mean levels of CD40 and CD86 expression (left) and IL-12 secretion (right) in BMDC from C57Bl/6 (□) and FcγR KO mice (▪) activated with IgG-IC overnight. (n=5). b. Proliferation of CD4⁺ T cells cultured with BMDC from C57Bl/6 (□) and FcγR KO mice (▪) loaded with IgG-IC (n=4). c. Tumor recurrence in untreated mice (∘), mice treated with WT BMDC loaded with IgG-IC (▪), or mice treated with FcγR KO BMDC loaded with IgG-IC (⋄) (n=4). d. and e. Percentages of tumor-free mice following adoptive transfer of 5×10⁶ splenic CD4⁴ T cells (left graph) or CD8⁺ T cells (right graph) from naive mice (●), or from LMP (a)- or B16 (b)-resected mice treated with DC+IgG_{C57} IC (▴), DC+lgM_{C57} IC (Δ), DC+IgG₁₂₉ IC (▪), or DC+lgM₁₂₉ IC (□), and subsequently challenged with LMP (a) or B16 (b) (n=6). f. Left: Tumor frequency in mice untreated (∘) or treated with DC loaded with IC formed with allogG and cytosolic tumor proteins (⋄), nuclear tumor proteins (Δ) or membrane tumor proteins (▪). Right: Tumor frequency in mice untreated (∘), treated with DC loaded with IC formed from alloIgG and membrane proteins (□), membrane proteins without O- and N-gycans (Δ), or heat-denatured membrane proteins (♦). (n=5). g. B16 tumor growth in C57Bl/6 mice untreated (∘), or injected with TNFα+CD40L (Δ), TNFα+CD40L+alloIgG (▪), or TNFα+CD40L and alloIgG absorbed on normal cells of the IgG-donor background (♦) or on normal cells of the tumor background (▪) (n=6). h. Tumor recurrence rates following resection in mice left untreated (∘), treated with 2×10⁶ DC loaded with IgG-IC from conventionally-raised C57Bl/6 (⋄), or with 2×10⁶ DC loaded with IgG-IC from gnotobiotic C57Bl/6 mice (▪). (n=4). i. B16 frequency in mice untreated (∘), or treated with BMDC loaded with intact B16 cells coated with alloIgG (□), or with intact B16 cells cross-linked to syngeneic IgG (▴). (n=4). j. B16 tumor frequency in mice untreated (∘) or treated with BMDC loaded with intact B16 cells coated with alloIgG (□) or with intact B16 coated with monoclonal IgG against MHC-I (▴).

FIG. 7a-7d. a. Sorting and culture schema of DC from BM and tumor. b. Mean levels of IL-12 (left graph) and TNFα (right graph) in the supematants of DC cultured overnight in medium alone (open bars), with B16 lysates (gray bars), or with alloIgG-IC (solid bars). c. Percentage of MHCII/CD86 cells (left panel) or CFSE levels (right panel) in tumor-associated DC following overnight activation with PBS (left barfor each condition) or CFSE-labeled alloIgG-IC (right barfor each condition) with or without stimulatory molecules. d. Flow cytometric analysis and confocal images of B16-derived DC cultured overnight with CFSE-labeled fixed B16 cells. Results represent mean values from at least 4 experiments±SEM. Asterisk (*) denotes p<0.05 and two asterisks (**) denotes p<0.01.

FIG. 8a-8h. a. B16 tumor size in C57Bl/6 mice left untreated (∘) or injected intratumorally with 129S1 alloIgG (⋄), LPS (□), TNFα+CD28 (Δ), LPS+alloIgG (▪) or TNFα+CD28+alloIgG (▴). b. B16 tumor size in C57Bl/6 mice left untreated (∘) or injected intratumorally with 1291 alloIgG (●), TNFα (□), CD28 (Δ), CD40L c. LL/2 tumor size in C57Bl/6 mice left untreated (∘), or injected intratumorally with 129S1 alloIgG (●), TNFα+CD40L (□), TNFα+CD28 (Δ), 129S1 alloIgG+TNFα+CD40L (▪) or TNFα+CD28+129S1 IgG (▴). d. Representative flow cytometric analysis of IgG binding total myeloid cells in B16 tumor-bearing mice 3 hours after intratumoral injection of PBS or 5 μg PE-labeled alloIgG (n=6). e. Total numbers of CD11c cells in the draining lymph nodes of B16 tumor-bearing mice 4 days after treatment (n=6). f. B16 growth in mice vaccinated with 2×10⁶ B cells, NK cells, mast cells or macrophages from B16 tumors untreated (∘), or injected with alloIgG (□) or alloIgG+TNFα+CD40L (⋄) (n=5). g. H&E sections of lung metastases on day 30 (magnitude ×10, n=7). h. Widefield microscopy of TADC from a lung carcinoma patient incubated overnight with autologous tumor cells (green) coated with selfIgG or alloIgG derived from a pool of 10 donors (1 μg/2×10⁵ cells) and in the presence of 50 ng/mL TNFα and 1 μg/mL CD40L.

FIG. 9a-9b. a. CD115+monocytes were isolated from the peripheral blood of mice and cultured for 5-7 days with GM-SCF to obtain DC. DC were than cultured with B16 tumor cells, or with B16 tumor cells pre-coated with allogeneic IgG. In some cases, 10 ng/mL TLR3 agonist (polyinosinic:polycytidylic acid (Poly I:C)) or 20 ng/mLTLR-9 agonist (CpG ODN) was also present. Shown are the mean percentages of DC expressing both CD40 and CD86. b. CD14+human monocytes were isolated from the peripheral blood of 3 healthy donors and cultured for 5-7 days with GM-SCF and IL-4 to obtain DC. DC were then cultured with PANC-1 tumor cells, or with PANC-1 tumor cells pre-coated with allogeneic IgG. In some cases, 10 ng/mL TLR3 agonist (polyinosinic:polycytidylic acid (Poly L:C)) or 1.5 μM calcium channel opener (ionomycin) was also present. Shown are the mean percentages of DC expressing both CD40 and CD86.

FIG. 10. Monoclonal allogeneic anti-MHC I antibody in combination with DC stimuli induces complete tumor regression. 4×10⁶ C.T26 colon cancer cells were injected s.c. into Balb/c mice above the right flank. Once tumors reached 25 mm², they were left untreated (open circles), injected intratumorally with TNFα+aCD40 agonist+allogeneic IgG (open squares), or with TNFα+aCD40 agonist+aH-2K^{d} IgG (an anti-MHC class I antibody)(solid squares).

FIG. 11a-11c. Immune cell infiltrate in tumors following therapy. Mice were injected s.c. with 2×10⁵ B16 melanoma cells which were allowed to grow until tumors reached 25 mm². Mice were then injected intratumorally with PBS (untreated), with TNFα+aCD40 alone, or with the combination of TNFα+aCD40+allogeneic IgG (from 129S1 mice), or TNFα+aCD40+antibody to Transmembrane Glycoprotein-NMB (TG-NMB, GPNMB). In some cases, mice lacking functional Fcg receptor signaling were injected with TNFα+aCD40+allogeneic IgG. After 6 days, tumors were excised and the entire cellular composition, including tumor cells, was tested by flow cytometry (n=8). a. Y axis is % CD45 cells among total tumor cells. b. Y axis is % INFg⁺ CD44⁺ cells among CD45⁺ cells (quantified for CD8 T cells and for CD4 T cells). c. Y axis is % of CD8⁺ cells expressing gp100 tetramer and % of CD8⁺ cells expressing Trp2 tetramer.

FIG. 12. Effect of adoptive transfer of T cells from treated mice on tumor development in naive mice. Splenic T cells were purified from B16-bearing mice, 6 days following their treatment with PBS (untreated), withTNFa+aCD40, or TNFa+aCD40 in combination with allogeneic IgG (alloIgG) or in combination with antibody to Transmembraine Glycoprotein-NMB (TG-NMB; GPNMB). 5×10⁶ CD4⁺ cells (Top) or CD8⁺ cells (Bottom) were injected i.v. into naive mice followed 1 hour later by s.c injection of 2.5×10⁵ B16 cells.

FIG. 13. Representative FACS plots from B16 tumors 6 days after treatment. Numbers represent % of positive cells.

FIG. 14. Representative FACS plots from B16 tumors 6 days after treatment.

FIG. 15a-15b. a. B6 cells were fixed and incubated for 1 hr with different allogeneic IgG subclasses to form immune complexes (IC). IC were added to BMDC cultures from wild type (WT) and FCγR knockout (KO) mice and DC expression of MHCII and CD86 was tested. b. C57Bl/6 mice were injected s.c. with B16 melanoma tumor cells. The tumors were resected at day 16 and used to form alloIgG-ICs with different alloIgG subclass antibodies. The ICs were cultured overnight with syngeneic BMDC which were then injected into the corresponding mice from which the tumor was removed.

### DETAILED DESCRIPTION OF THE EMBODIMENTS

### I. Introduction

Described herein are methods, compositions, and kits for treatment of cancer. Some of the methods, compositions, and kits are based on a discovery by the inventors that the combination of a cancer cell bridging agent and antigen presenting cell (APC) stimulation is surprisingly effective at treating cancer. In some embodiments, the APC is a dendritic cell.

The cancer cell bridging agent is an agent that bridges between an antigen on the cancer cell and one or more receptors on the antigen presenting cell. Generally, the bridging agent is an antibody. In some cases, the bridging agent is an antibody that recognizes one or more antigens on the surface of the cancer cell. Typically, the antibody can bind to a cancer cell or associate with a tumor mass and be recognized by an Fc receptor on an APC. In some embodiments, the antibody is an allogeneic antibody (an alloantibody). In one embodiment, the antibody is an IgG antibody, e.g., an allogeneic IgG antibody.

Although APC are often inhibited in the context of a tumor, the use of an APC stimulatory agent can overcome tumor-induced inhibition. Additionally, or in the alternative, the APC stimulatory agent can activate the APC to a greater extent than would otherwise occur. The activated APC can thus recognize the bridging agent bound to the cancer antigen and internalize the cancer cell, or a portion thereof. The APC can then generate and present numerous antigens from the cancer cell to CD4 and CD8 T-cells, thus activating T-cells against numerous antigens expressed by the cancer cells. This surprisingly robust activation of T-cells against multiple tumor antigens, which antigens do not have to be predetermined prior to treatment, dramatically decreases the likelihood that the tumor can escape recognition or destruction by the immune system.

Anti-tumor antibodies can promote tumor growth or progression or induce T-cell tolerance to a tumor. See, e.g., Cancer Cell 2005 v7 p411; Cancer Cell 2010 v17 p121; J Exp Med. 2008 Jul. 7; 205(7):1687-700); and references cited therein. Intravenous application of anti-tumor IgG antibodies is not generally an effective cancer treatment. See, e.g., Ann. NY Acad Sci 2007 v1110 p305-314. Similarly, stimulation of antigen presenting cells has been shown to promote tumor growth or progression. See, e.g., Oncotarget. 2014 Dec. 15; 5(23):12027-42; Cancer Biol Ther. 2014 January; 15(1):99-107. Therefore, the robust anti-tumor response induced by the combination of a bridging agent (e.g., antibody or alloantibody, such as an allogeneic IgG) and APC stimulation (e.g., dendritic cell stimulation) is a surprising and unexpected result. Moreover, unlike other successful antibody-based cancer treatments, this effect does not primarily result from, or require, antibody mediated interference with cancer cell signaling or antibody dependent cellular cytotoxicity.

Aspects of the methods include administering to an individual (e.g., an individual having cancer): (i) an antibody composition having an allogeneic IgG antibody that specifically binds to an antigen of a cancer cell of the individual; and (ii) a treatment that activates an APC of the individual, wherein the APC is a dendritic cell, a macrophage, or a B-cell. Other embodiments include administering to an individual a population of APCs exposed to a tumor antigen in the presence of (i) an antibody composition having an allogeneic IgG antibody that specifically binds to an antigen of a cancer cell of the individual; and (ii) a treatment that activates an APC of the individual, wherein the APC is a dendritic cell, a macrophage, or a B-cell. In some cases, the antibody composition includes polyclonal allogeneic IgG antibodies with a plurality of binding specificities.

In some cases, the treatment that activates an APC, e.g., a dendritic cell (DC), of the individual includes administering to the individual a stimulatory composition having an APC stimulatory agent (e.g., a DC stimulatory agent). In some cases, an APC stimulatory agent (e.g., a DC stimulatory agent) is conjugated to an allogeneic IgG antibody. For example, the APC stimulatory agent can be conjugated to an allogeneic antibody such that it is likely to be labile. In some cases, the conjugation is labile after internalization by the APC. For instance, the APC stimulatory agent can be conjugated to the allogeneic antibody, internalized by an APC, and the APC stimulatory agent released from the antibody, thereby stimulating the APC. In some cases, the conjugation is labile under conditions likely to be encountered at or near a tumor cell, or when bound to the surface of an APC. For example, the stimulatory agent can be conjugated via an ester or peptide linkage that can be cleaved by one or more cell surface proteases or esterases. In some cases, the stimulatory agent, either alone or conjugated to an allogenic antibody, binds to a receptor on the surface of the APC. In some cases, the stimulatory composition includes CD40 ligand (CD40L) and a proinflammatory cytokine.

Methods are provided for inducing an immune response in an individual. Aspects of the methods include: (a) contacting in vitro an APC, e.g., DC, from the individual with a target antigen and an antibody composition having an allogeneic IgG antibody that specifically binds to the target antigen, at a dose and for a period of time effective for the uptake of the target antigen by the APC, e.g., DC, thereby producing a loaded APC, e.g., DC; and (b) contacting a T cell of the individual with the loaded APC, e.g., DC, where the loaded APC, e.g., DC, presents antigens to the T cell to produce a contacted T cell, and the contacted T cell generates an immune response specific to the presented antigens. In some cases, the APC, e.g., DC, is from an individual with cancer and the target antigen is associated with the cancer. In some cases, the APC, e.g., DC, is contacted with a cancer cell from the individual. In some cases, the APC, e.g., DC, is contacted with a lysate from cancer cells of the individual. In some cases, the APC, e.g., DC, is contacted with two or more plasma membrane proteins (which can be part of alysate) from cancer cells of the individual. In some cases, the APC, e.g., DC, is contacted with a stimulatory composition comprising an APC stimulatory agent (e.g., dendritic cell stimulatory agent). In some cases, the stimulatory composition comprises a CD40L and a proinflammatory cytokine. In some cases, the dendritic cell stimulatory agent is conjugated to an allogeneic IgG antibody. In some cases, the target antigen is contacted with the antibody composition prior to contacting the APC, e.g., DC. In some cases, the APC, e.g., DC, is simultaneously contacted with the target antigen and the antibody composition. In some cases, the step of contacting a T cell is performed in vivo and the method comprises introducing the loaded APC, e.g., DC, into the individual. In some cases, the step of contacting a T cell is performed in vitro and the method comprises introducing the contacted T cell into the individual.

Compositions and kits for practicing the methods of the disclosure are also provided. In some cases, a subject composition includes: polyclonal allogeneic IgG antibodies with a plurality of binding specificities; and at least one APC stimulatory agent (e.g., dendritic cell stimulatory agent). In some cases, a subject composition includes: polyclonal allogeneic IgG antibodies with a plurality of binding specificities; a CD40L; and a proinflammatory cytokine (e.g., TNFα, IL-1α, IL-1β, IL-19, interferon gamma (IFNγ), and the like). In some cases, an APC stimulatory agent (e.g., dendritic cell stimulatory agent) is conjugated to at least one of the allogeneic IgG antibodies of the composition. In some cases, a subject composition includes intravenous immunoglobulin (IVIG) or antibodies purified or enriched from IVIG.

Before the present methods and compositions are described, it is to be understood that this invention is not limited to particular method or composition described, as such may, of course, vary. It is also to be understood that the terminology used herein is for the purpose of describing particular embodiments only, and is not intended to be limiting, since the scope of the present invention will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the invention. The upper and lower limits of these smaller ranges may independently be included or excluded in the range, and each range where either, neither or both limits are included in the smaller ranges is also encompassed within the invention, subject to any specifically excluded limit in the stated range. Where the stated range includes one or both of the limits, ranges excluding either or both of those included limits are also included in the invention.

Unless defined otherwise, all technical and scientific terms used herein have the same meaning as commonly understood by one of ordinary skill in the art to which this invention belongs. Although any methods and materials similar or equivalent to those described herein can be used in the practice or testing of the present invention, some potential and preferred methods and materials are now described. All publications mentioned herein are incorporated herein by reference to disclose and describe the methods and/or materials in connection with which the publications are cited. It is understood that the present disclosure supercedes any disclosure of an incorporated publication to the extent there is a contradiction.

As will be apparent to those of skill in the art upon reading this disclosure, each of the individual embodiments described and illustrated herein has discrete components and features which may be readily separated from or combined with the features of any of the other several embodiments without departing from the scope or spirit of the present invention. Any recited method can be carried out in the order of events recited or in any other order which is logically possible.

It must be noted that as used herein and in the appended claims, the singular forms “a”, “an”, and “the” include plural referents unless the context clearly dictates otherwise. Thus, for example, reference to “a cell” includes a plurality of such cells and reference to “the peptide” includes reference to one or more peptides and equivalents thereof, e.g. polypeptides, known to those skilled in the art, and so forth.

The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates which may need to be independently confirmed

### II. Definitions

The terms “specific binding,” “specifically binds,” and the like, refer to non-covalent or covalent preferential binding to a molecule relative to other molecules or moieties in a solution or reaction mixture (e.g., an antibody specifically binds to a particular polypeptide or epitope relative to other available polypeptides). For example, a subject allogeneic IgG antibody that specifically binds to an antigen (a target antigen) of a cancer cell preferentially binds to that particular antigen relative to other available antigens. However, the target antigen need not be specific to the cancer cell or even enriched in cancer cells relative to other cells (e.g., the target antigen can be expressed by other cells). Thus, in the phrase “an allogeneic antibody that specifically binds to an antigen of a cancer cell,” the term “specifically” refers to the specificity of the antibody and not to the uniqueness of the antigen in that particular cell type. In some embodiments, the affinity of one molecule for another molecule to which it specifically binds is characterized by a K_{D} (dissociation constant) of 10⁻⁵ M or less (e.g., 10⁻⁶ M or less, 10⁻⁷ M or less, 10⁻⁸ M or less, 10⁻⁹ M or less, 10⁻¹⁰ M or less, 10⁻¹¹ M or less, 10⁻¹² M or less, 10⁻¹³ M or less, 10⁻¹4 M or less, 10⁻¹⁵ M or less, or 10⁻¹⁶ M or less). “Affinity” refers to the strength of binding. For example increased binding affinity can be indicated by alower K_{D}. In some cases, increased binding affinity is correlated with a lower K_{D}.

The term “specific binding member” as used herein refers to a member of a specific binding pair (i.e., two molecules, usually two different molecules, where one of the molecules, e.g., a first specific binding member, through non-covalent means specifically binds to the other molecule, e.g., a second specific binding member).

The term “specific binding agent” as used herein refers to any agent that specifically binds a biomolecule (e.g., a marker such as a nucleic acid marker molecule, a protein marker molecule, etc.). In some cases, a “specific binding agent” for a marker molecule (e.g., a dendritic cell marker molecule) is used. Specific binding agents can be any type of molecule. In some cases, a specific binding agent is an antibody or a fragment thereof. In some cases, a specific binding agent is a nucleic acid probe (e.g., an RNA probe; a DNA probe; an RNA/DNA probe; a modified nucleic acid probe, e.g., a locked nucleic acid (LNA) probe, a morpholino probe, etc.; and the like).

As used herein, a “marker molecule” does not have to be definitive (i.e., the marker does not have to definitely mark the cell as being of a particular type. For example, the expression of a marker molecule by a cell can be indicative (i.e., suggestive) that the cell is of a particular cell type. For example, if 3 cell types (type A, type B, and type C) express a particular marker molecule (e.g., a particular mRNA, a particular protein, etc.), expression of that marker molecule by a cell cannot necessarily be used by itself to definitively determine that the cell is a type A cell. However, expression of such a marker can suggest that the cell is a type A cell. In some cases, expression of such a marker, combined with other evidence, can definitively show that the cell is a type A cell. As another illustrative example, if a particular cell type is known to express two or more particular marker molecules (e.g., mRNAs, proteins, a combination thereof, etc.) then the expression by a cell of one of the two or more particular marker molecules can be suggestive, but not definitive, that the cell is of the particular type in question. In such a case, the marker is still considered a marker molecule.

“Antibody” refers to a polypeptide comprising an antigen binding region (including the complementarity determining region (CDRs)) from an immunoglobulin gene or fragments thereof that specifically binds and recognizes an antigen. The recognized immunoglobulin genes include the kappa, lambda, alpha, gamma, delta, epsilon, and mu constant region genes, as well as the myriad immunoglobulin variable region genes. Light chains are classified as either kappa or lambda. Heavy chains are classified as gamma, mu, alpha, delta, or epsilon, which in turn define the immunoglobulin classes, IgG, IgM, IgA, IgD and IgE, respectively. IgG antibodies are large molecules of about 150 kDa composed of four peptide chains. IgG antibodies contain two identical class γ heavy chains of about 50 kDa and two identical light chains of about 25 kDa, thus a tetrameric quaternary structure. The two heavy chains are linked to each other and to a light chain each by disulfide bonds. The resulting tetramer has two identical halves, which together form the Y-like shape. Each end of the fork contains an identical antigen binding site. There are four IgG subclasses (IgG1, 2, 3, and 4) in humans, named in order of their abundance in serum (IgG1 being the most abundant). Typically, the antigen-binding region of an antibody will be most critical in specificity and affinity of binding.

An exemplary immunoglobulin (antibody) structural unit comprises a tetramer. Each tetramer is composed of two identical pairs of polypeptide chains, each pair having one “light” (about 25 kD) and one “heavy” chain (about 50-70 kD). The N-terminus of each chain defines a variable region of about 100 to 110 or more amino acids primarily responsible for antigen recognition. The terms variable light chain (V_{L}) and variable heavy chain (V_{H}) refer to these light and heavy chains respectively.

Antibodies exist, e.g., as intact immunoglobulins or as a number of well-characterized fragments produced by digestion with various peptidases. Thus, for example, pepsin digests an antibody below the disulfide linkages in the hinge region to produce F(ab)′₂, a dimer of Fab which itself is a light chain joined to V_{H}-C_{H}1 by a disulfide bond. The F(ab)′₂ may be reduced under mild conditions to break the disulfide linkage in the hinge region, thereby converting the F(ab)′₂ dimer into an Fab′ monomer. The Fab′ monomer is essentially Fab with part of the hinge region (see Fundamental Immunology (Paul ed., 3d ed. 1993). While various antibody fragments are defined in terms of the digestion of an intact antibody, one of skill will appreciate that such fragments may be synthesized de novo either chemically or by using recombinant DNA methodology. Thus, the term antibody, as used herein, also includes antibody fragments either produced by the modification of whole antibodies, or those synthesized de novo using recombinant DNA methodologies (e.g., single chain Fv) or those identified using phage display libraries (see, e.g., McCafferty et al., Nature 348:552-554 (1990))

The term “antibody” is used in the broadest sense and specifically covers monoclonal antibodies (including full length monoclonal antibodies), polyclonal antibodies, multispecific antibodies (e.g., bispecific antibodies), and antibody fragments so long as they exhibit the desired biological activity. “Antibody fragment”, and all grammatical variants thereof, as used herein are defined as a portion of an intact antibody comprising the antigen binding site or variable region of the intact antibody, wherein the portion is free of the constant heavy chain domains (i.e. CH2, CH3, and CH4, depending on antibody isotype) of the Fc region of the intact antibody. Examples of antibody fragments include Fab, Fab′, Fab′-SH, F(ab′)₂, and Fv fragments; diabodies; any antibody fragment that is a polypeptide having a primary structure consisting of one uninterrupted sequence of contiguous amino acid residues (referred to herein as a “single-chain antibody fragment” or “single chain polypeptide”), including without limitation (1) single-chain Fv (scFv) molecules (2) single chain polypeptides containing only one light chain variable domain, or a fragment thereof that contains the three CDRs of the light chain variable domain, without an associated heavy chain moiety (3) single chain polypeptides containing only one heavy chain variable region, or a fragment thereof containing the three CDRs of the heavy chain variable region, without an associated light chain moiety; (4) nanobodies comprising single Ig domains from non-human species or other specific single-domain binding modules; and (5) multispecific or multivalent structures formed from antibody fragments. In an antibody fragment comprising one or more heavy chains, the heavy chain(s) can contain any constant domain sequence (e.g. CH1 in the IgG isotype) found in a non-Fc region of an intact antibody, and/or can contain any hinge region sequence found in an intact antibody, and/or can contain a leucine zipper sequence fused to or situated in the hinge region sequence or the constant domain sequence of the heavy chain(s).

As used in this disclosure, the term “epitope” means any antigenic determinant on an antigen to which the paratope of an antibody binds. Epitopic determinants usually consist of chemically active surface groupings of molecules such as amino acids or sugar side chains and usually have specific three dimensional structural characteristics, as well as specific charge characteristics.

The terms “polypeptide,” “peptide” and “protein” are used interchangeably herein to refer to a polymer of amino acid residues. The terms also apply to amino acid polymers in which one or more amino acid residue is an artificial chemical mimetic of a corresponding naturally occurring amino acid, as well as to naturally occurring amino acid polymers and non-naturally occurring amino acid polymer.

As used herein, the term “APC” or “antigen presenting cell” refers to a cell that expresses major histocompatibility complex class II (MHC class II) proteins on its cell membrane surface and is capable of presenting antigens in complex with MHC class II to T-cells, thereby activating T-cells to the presented antigens. In some embodiments, the APC is a dendritic cell. In some embodiments, the APC is a macrophage. In some embodiments, the APC is a B-cell. In some embodiments, the APC is a dendritic cell, macrophage, or B-cell. In some embodiments, the APC is a dendritic cell or a macrophage. In some embodiments, the APC is a dendritic cell or a B-cell. In some cases, the APC is not a macrophage. In some cases, the APC is not a B-cell.

The terms “passaging” or “passage” (i.e., splitting or split) in the context of cell culture are known in the art and refer to the transferring of a small number of cells into a new vessel. Cells can be cultured if they are split regularly because it avoids the senescence associated with high cell density. For adherent cells, cells are detached from the growth surface as part of the passaging protocol. Detachment is commonly performed with the enzyme trypsin and/or other commercially available reagents (e.g., TrypLE, EDTA (Ethylenediaminetetraacetic acid), a policemen (e.g., a rubber policemen) for physically scrapping the cells from the surface, etc.). A small number of detached cells (e.g., as few as one cell) can then be used to seed a new cell population, e.g., after dilution with additional media. Therefore, to passage a cell population means to dissociate at least a portion of the cells of the cell population, dilute the dissociated cells, and to plate the diluted dissociated cells (i.e., to seed a new cell population).

The terms “media” and “medium” are herein used interchangeably. Cell culture media is the liquid mixture that baths cells during in vitro culture.

The term “population”, e.g., “cell population” or “population of cells”, as used herein means a grouping (i.e., a population) of two or more cells that are separated (i.e., isolated) from other cells and/or cell groupings. For example, a 6-well culture dish can contain 6 cell populations, each population residing in an individual well. The cells of a cell population can be, but need not be, clonal derivatives of one another. A cell population can be derived from one individual cell. For example, if individual cells are each placed in a single well of a 6-well culture dish and each cell divides one time, then the dish will contain 6 cell populations. A cell population can be any desired size and contain any number of cells greater than one cell. For example, a cell population can be 2 or more, 10 or more, 100 or more, 1,000 or more, 5,000 or more, 10⁴ or more, 10⁵ or more, 10⁶ or more, 10⁷ or more, 10⁸ or more, 10⁹ or more, 10¹⁰ or more, 10¹¹ or more, 10¹² or more, 10¹³ or more, 10¹⁴ or more, 10¹⁵ or more, 10¹⁶ or more, 10¹⁷ or more, 10¹⁸ or more, 10¹⁹ or more, or 10²⁰ or more cells.

The term “plurality” as used herein means greater than one. For example, a plurality can be 2 or more, 5 or more, 10 or more, 25 or more, 50 or more, 100 or more, 500 or more, 1,000 or more, 2,000 or more, 5,000 or more, 10⁴ or more, 10⁵ or more, 10⁶ or more, 10⁷ or more, etc. For example, an antibody composition having polyclonal allogeneic IgG antibodies with a plurality of binding specificities is a composition of allogeneic IgG antibodies, where two or more (e.g., 5 or more, 10 or more, 25 or more, 50 or more, 100 or more, 500 or more, 1,000 or more, 2,000 or more, 5,000 or more, 10⁴ or more, 10⁵ or more, 10⁸ or more, 10⁷ or more) of the antibodies have different binding specificities (e.g., specifically bind to different epitopes of the same antigen, specifically bind to different antigens, and the like).

### III. Methods and Compositions

Aspects of the disclosure include methods and compositions for inducing an immune response in an individual. Because such methods can be used to treat an individual, such methods can also be referred to as methods of treating an individual.

In some embodiments, methods of treating an individual having cancer include administering to the individual: (i) an antibody composition (as described in detail above) that includes an allogeneic IgG antibody that specifically binds to an antigen of a cancer cell of the individual; and (ii) a treatment that activates antigen presenting cells (APC), e.g., dendritic cells (DC), of the individual. In such cases, uptake of target antigens (e.g., cancer cells, cancer cell debris, secreted cancer cell antigens, etc.) by endogenous APCs, e.g., dendritic cells, of the individual is induced.

In some embodiments, methods of treating an individual (e.g., an individual having cancer) include administering to the individual: (i) an antibody composition that includes polyclonal allogeneic IgG antibodies with a plurality of binding specificities; and (ii) a treatment that activates antigen presenting cells (APC), e.g., dendritic cells (DC), of the individual. In some embodiments, methods of treating an individual (e.g., an individual having cancer) include administering to the individual: (i) an antibody composition that includes an allogeneic IgG antibody that specifically binds to an antigen of a cancer cell of the individual; (ii) a CD40 ligand (CD40L); and (iii) a proinflammatory cytokine.

In some embodiments, methods of inducing an immune response in an individual include: (a) contacting in vitro an APC, e.g., DC, from the individual with: (i) a target antigen; and (ii) an antibody composition comprising an allogeneic IgG antibody that specifically binds to the target antigen, at a dose and for a period of time effective for the uptake of the target antigen by the APC, e.g., DC, thereby producing a loaded APC, e.g., DC; and (b) contacting a T cell of the individual with the loaded APC, e.g., DC, wherein the loaded APC, e.g., DC, presents antigens to the T cell to produce a contacted T cell, and the contacted T cell generates an immune response specific to the presented antigens. In some cases, the target antigen (e.g., a target cell) is contacted with the antibody composition, producing an immune complex, prior to contacting the APC, e.g., DC. Thus, in some cases, the methods include contacting an APC, e.g., DC, with an immune complex. In some cases, the step of contacting a T cell of the individual is in vivo. In some cases, the step of contacting a T cell of the individual is in vitro.

In some embodiments, methods of inducing an immune response in an individual include: (a) contacting in vitro an APC, e.g., DC, from the individual with: (i) a target antigen; and (ii) an antibody composition comprising an allogeneic IgG antibody that specifically binds to the target antigen, at a dose, under conditions, and for a period of time effective for the uptake of the target antigen by the APC, e.g., DC, thereby producing a loaded APC, e.g., DC; and (b) contacting a T cell of the individual with the loaded APC, e.g., DC, wherein the loaded APC, e.g., DC, presents antigens to the T cell to produce a contacted T cell, and the contacted T cell generates an immune response specific to the presented antigens. In some cases, the target antigen (e.g., a target cell) is contacted with the antibody composition, producing an immune complex, prior to contacting the APC, e.g., DC. Thus, in some cases, the methods include contacting an APC, e.g., DC, with an immune complex. In some cases, the step of contacting a T cell of the individual is in vivo. In some cases, the step of contacting a T cell of the individual is in vitro.

The terms “treatment”, “treating”, “treat” and the like are used herein to generally refer to obtaining a desired pharmacologic and/or physiologic effect. The effect can be prophylactic in terms of completely or partially preventing a disease or symptom(s) thereof and/or may be therapeutic in terms of a partial or complete stabilization or cure for a disease and/or adverse effect attributable to the disease. The term “treatment” encompasses any treatment of a disease in a mammal, particularly a human, and includes: (a) preventing the disease and/or symptom(s) from occurring in a subject who may be predisposed to the disease or symptom(s) but has not yet been diagnosed as having it; (b) inhibiting the disease and/or symptom(s), i.e., arresting development of a disease and/or the associated symptoms; or (c) relieving the disease and the associated symptom(s), i.e., causing regression of the disease and/or symptom(s). Those in need of treatment can include those already inflicted (e.g., those with cancer, e.g. those having tumors) as well as those in which prevention is desired (e.g., those with increased susceptibility to cancer; those with pre-cancerous tumors, lesions; those suspected of having cancer; etc.).

The terms “recipient”, “individual”, “subject”, “host”, and “patient”, are used interchangeably herein and refer to any mammalian subject for whom diagnosis, treatment, or therapy is desired, particularly humans. “Mammal” for purposes of treatment refers to any animal classified as a mammal, including humans, domestic and farm animals, and zoo, sports, or pet animals, such as dogs, horses, cats, cows, sheep, goats, pigs, camels, etc. In some embodiments, the mammal is human.

A therapeutic treatment is one in which the subject is inflicted prior to administration and a prophylactic treatment is one in which the subject is not inflicted prior to administration. In some embodiments, the subject has an increased likelihood of becoming inflicted or is suspected of having an increased likelihood of becoming inflicted (e.g., relative to a standard, e.g., relative to the average individual, e.g., a subject may have a genetic predisposition to cancer and/or a family history indicating increased risk of cancer), in which case the treatment can be a prophylactic treatment. In some cases, the term “vaccination” is used to describe a prophylactic treatment. For example, in some cases where the subject being treated has not been diagnosed as having cancer (e.g., the subject has an increased likelihood of becoming inflicted, is suspected of having an increased likelihood of becoming inflicted)(e.g., a subject may have a genetic predisposition to cancer and/or a family history indicating increased risk of cancer), the subject can be vaccinated (treated such that the treatment is a prophylactic treatment) by performing one or more of the subject methods (e.g., administration of (i) an antibody composition that comprises polyclonal allogeneic IgG antibodies with a plurality of binding specificities; and (ii) a treatment that activates APC, e.g., DC, of the individual (e.g., administering to the individual a APC stimulatory composition, e.g., dendritic cell stimulatory composition, having an APC stimulatory agent, e.g., dendritic cell stimulatory agent).

APC stimulatory agents include, but are not limited to dendritic cell stimulatory agents, macrophage stimulatory agents, or B-cell stimulatory agents. In some cases, the APC stimulatory agent is a dendritic cell stimulatory agent. In some cases, the APC stimulatory agent is a macrophage stimulatory agent. In some cases, the APC stimulatory agent is a B-cell stimulatory agent. In some cases, the APC stimulatory agent is not a macrophage stimulatory agent.

In some cases, the APC stimulatory composition includes a dendritic cell stimulatory agent and a B-cell stimulatory agent. In some cases, the APC stimulatory composition includes a dendritic cell stimulatory agent but does not include a macrophage stimulatory agent. In some cases, the APC stimulatory composition includes at least two dendritic cell stimulatory agents.

A dendritic cell stimulatory composition can include, but is not limited to a composition that contains (i) a Toll-like receptor (TLR) agonist; (ii) a CD40 agonist and a proinflammatory cytokine; (iii) a checkpoint molecule neutralizing compound; (iv) an indoleamine 2,3-dioxygenase (IDO) inhibitor (v) an NFkB activator; (vi) a compound that opens calcium channels; (vii) a T cell-related co-stimulatory molecule; or (vIII) a combination thereof.

A B-cell stimulatory composition can include, but is not limited to, a composition that contains (i) a Toll-like receptor (TLR) agonist; (ii) a CD40 agonist; (iii) a CD40 agonist and a proinflammatory cytokine; (iv) an antigen that binds the B-cell receptor; (v) an anti-idiotype antibody; (vi) or an agent that cross-links surface immunoglobulin. In some cases, the proinflammatory cytokine is IL-1, IL-2, IL-3, IL-4, IL-6, IL-7, IL-9, IL-10, IL-12, IL-15, IL-18, IL-21, IFN-α, IFN-β, IFN-γ, G-CSF, or GM-CSF. In some cases, the TLR agonist is CpG ODN, immunostimulatory DNA, immunostimulatory RNA, immunostimulatory oligonucleotides, Imiquimod, Resiquimod, Loxribine, Flagellin, FSL-1 or LPS. In some cases, the antigen is a self antigen, an allogeneic antigen, a peptide antigen, a nucleic acid antigen, a carbohydrate antigen, or a tumor associated antigen. In some cases, the agent that cross-links surface immunoglobulin is an anti-Ig antibody, an anti-idiotype antibody, or an anti-isotype antibody.

In some cases where the subject being treated has not been diagnosed as having cancer (e.g., the subject has an increased likelihood of becoming inflicted, is suspected of having an increased likelihood of becoming inflicted)(e.g., a subject may have a genetic predisposition to cancer and/or a family history indicating increased risk of cancer), the subject can be vaccinated (treated such that the treatment is a prophylactic treatment) by performing one or more of the subject methods (e.g., administration of (i) an antibody composition that comprises polyclonal allogeneic IgG antibodies with a plurality of binding specificities; and (ii) a treatment that activates APC, e.g., DC, of the individual (e.g., administering to the individual a APC stimulatory composition, e.g., dendritic cell stimulatory composition, having an APC stimulatory agent, e.g., dendritic cell stimulatory agent, such as, e.g., a dendritic cell stimulatory composition that includes (i) a Toll-like receptor (TLR) agonist; (ii) a CD40 agonist and a proinflammatory cytokine; (iii) a checkpoint molecule neutralizing compound; (iv) an indoleamine 2,3-dioxygenase (IDO) inhibitor; (v) an NFkB activator; (vi) a compound that opens calcium channels; (vii) a T cell-related co-stimulatory molecule; or (vIII) a combination thereof).

The terms “co-administration” and “in combination with” include the administration of two or more therapeutic agents either simultaneously, concurrently or sequentially within no specific time limits. In one embodiment, the agents are present in the cell or in the subject's body at the same time or exert their biological or therapeutic effect at the same time. In one embodiment, the therapeutic agents are in the same composition or unit dosage form. In other embodiments, the therapeutic agents are in separate compositions or unit dosage forms. In certain embodiments, a first agent can be administered prior to (e.g., minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of a second therapeutic agent.

In some embodiments, the individual to be treated is an individual with cancer. As used herein “cancer” includes any form of cancer (e.g., leukemia; acute myeloid leukemia (AML); acute lymphoblastic leukemia (ALL); lymphomas; mesothelioma (MSTO); minimal residual disease; solid tumor cancers, e.g., lung, prostate, breast, bladder, colon, ovarian, pancreas, kidney, glioblastoma, medulloblastoma, leiomyosarcoma, and head & neck squamous cell carcinomas, melanomas; etc.), including both primary and metastatic tumors; and the like. In some cases, the individual has recently undergone treatment for cancer (e.g., radiation therapy, chemotherapy, surgical resection, etc.) and are therefore at risk for recurrence. Any and all cancers are suitable cancers to be treated by the subject methods, compositions, and kits.

The terms “cancer,” “neoplasm,” and “tumor” are used interchangeably herein to refer to cells which exhibit autonomous, unregulated growth, such that they exhibit an aberrant growth phenotype characterized by a significant loss of control over cell proliferation. Cells of interest for detection, analysis, and/or treatment in the present application include precancerous (e.g., benign), malignant, pre-metastatic, metastatic, and non-metastatic cells. Cancers of virtually every tissue are known. The phrase “cancer burden” refers to the quantum of cancer cells or cancer volume in a subject. Reducing cancer burden accordingly refers to reducing the number of cancer cells or the cancer volume in a subject. The term “cancer cell” as used herein refers to any cell that is a cancer cell or is derived from a cancer cell e.g. clone of a cancer cell. The term also includes a portion of a cancer cell, such as a sub-cellular portion, a cell membrane portion, or a cell lysate of a cancer cell. Many types of cancers are known to those of skill in the art, including solid tumors such as carcinomas, sarcomas, glioblastomas, melanomas, lymphomas, myelomas, etc., and circulating cancers such as leukemias.

As used herein “cancer” includes any form of cancer, including but not limited to solid tumor cancers (e.g., lung, prostate, breast, bladder, colon, ovarian, pancreas, kidney, liver, glioblastoma, medulloblastoma, leiomyosarcoma, head & neck squamous cell carcinomas, melanomas, neuroendocrine; etc.) and liquid cancers (e.g., hematological cancers); carcinomas; soft tissue tumors; sarcomas; teratomas; melanomas; leukemias; lymphomas; and brain cancers, including minimal residual disease, and including both primary and metastatic tumors. Any cancer is a suitable cancer to be treated by the subject methods and compositions. In some cases, the cancer cells express PD-L1. In some cases, the cancer cells do not express PD-L1 (e.g., in such cases, cells of the immune system of the individual being treated express PD-L1).

Carcinomas are malignancies that originate in the epithelial tissues. Epithelial cells cover the external surface of the body, line the internal cavities, and form the lining of glandular tissues. Examples of carcinomas include, but are not limited to: adenocarcinoma (cancer that begins in glandular (secretory) cells), e.g., cancers of the breast, pancreas, lung, prostate, and colon can be adenocarcinomas; adrenocortical carcinoma; hepatocellular carcinoma; renal cell carcinoma; ovarian carcinoma; carcinoma in situ; ductal carcinoma; carcinoma of the breast; basal cell carcinoma; squamous cell carcinoma; transitional cell carcinoma; colon carcinoma; nasopharyngeal carcinoma; multilocular cystic renal cell carcinoma; oat cell carcinoma; large cell lung carcinoma; small cell lung carcinoma; non-small cell lung carcinoma; and the like. Carcinomas may be found in prostrate, pancreas, colon, brain (usually as secondary metastases), lung, breast, skin, etc.

Soft tissue tumors are a highly diverse group of rare tumors that are derived from connective tissue. Examples of soft tissue tumors include, but are not limited to: alveolar soft part sarcoma; angiomatoid fibrous histiocytoma; chondromyoxid fibroma; skeletal chondrosarcoma; extraskeletal myxoid chondrosarcoma; clear cell sarcoma; desmoplastic small round-cell tumor; dermatofibrosarcoma protuberans; endometrial stromal tumor; Ewing's sarcoma; fibromatosis (Desmoid); fibrosarcoma, infantile; gastrointestinal stromal tumor; bone giant cell tumor; tenosynovial giant cell tumor; inflammatory myofibroblastic tumor; uterine leiomyoma; leiomyosarcoma; lipoblastoma; typicallipoma; spindle cell or pleomorphic lipoma; atypical lipoma; chondroid lipoma; well-differentiated liposarcoma; myxoid/round cell liposarcoma; pleomorphic liposarcoma; myxoid malignant fibrous histiocytoma; high-grade malignant fibrous histiocytoma; myxofibrosarcoma; malignant peripheral nerve sheath tumor, mesothelioma; neuroblastoma; osteochondroma; osteosarcoma; primitive neuroectodermal tumor; alveolar rhabdomyosarcoma; embryonal rhabdomyosarcoma; benign or malignant schwannoma; synovial sarcoma; Evan's tumor; nodular fasciitis; desmoid-type fibromatosis; solitary fibrous tumor; dermatofibrosarcoma protuberans (DFSP); angiosarcoma; epithelioid hemangioendothelioma; tenosynovial giant cell tumor (TGCT); pigmented villonodular synovitis (PVNS); fibrous dysplasia; myxofibrosarcoma; fibrosarcoma; synovial sarcoma; malignant peripheral nerve sheath tumor; neurofibroma; and pleomorphic adenoma of soft tissue; and neoplasias derived from fibroblasts, myofibroblasts, histiocytes, vascular cells/endothelial cells and nerve sheath cells.

A sarcoma is a rare type of cancer that arises in cells of mesenchymal origin, e.g., in bone or in the soft tissues of the body, including cartilage, fat, muscle, blood vessels, fibrous tissue, or other connective or supportive tissue. Different types of sarcoma are based on where the cancer forms. For example, osteosarcoma forms in bone, liposarcoma forms in fat, and rhabdomyosarcoma forms in muscle. Examples of sarcomas include, but are not limited to: askin's tumor; sarcoma botryoides; chondrosarcoma; ewing's sarcoma; malignant hemangioendothelioma; malignant schwannoma; osteosarcoma; and soft tissue sarcomas (e.g., alveolar soft part sarcoma; angiosarcoma; cystosarcoma phyllodesdermatofibrosarcoma protuberans (DFSP); desmoid tumor; desmoplastic small round cell tumor; epithelioid sarcoma; extraskeletal chondrosarcoma; extraskeletal osteosarcoma; fibrosarcoma; gastrointestinal stromal tumor (GIST); hemangiopericytoma; hemangiosarcoma (more commonly referred to as “angiosarcoma”); kaposi's sarcoma; leiomyosarcoma; liposarcoma; lymphangiosarcoma; malignant peripheral nerve sheath tumor (MPNST); neurofibrosarcoma; synovial sarcoma; undifferentiated pleomorphic sarcoma, and the like).

A teratoma is a type of germ cell tumor that may contain several different types of tissue (e.g., can include tissues derived from any and/or all of the three germ layers: endoderm, mesoderm, and ectoderm), including for example, hair, muscle, and bone. Teratomas occur most often in the ovaries in women, the testicles in men, and the tailbone in children.

Melanoma is a form of cancer that begins in melanocytes (cells that make the pigment melanin). It may begin in a mole (skin melanoma), but can also begin in other pigmented tissues, such as in the eye or in the intestines.

Leukemias are cancers that start in blood-forming tissue, such as the bone marrow, and causes large numbers of abnormal blood cells to be produced and enter the bloodstream. For example, leukemias can originate in bone marrow-derived cells that normally mature in the bloodstream. Leukemias are named for how quickly the disease develops and progresses (e.g., acute versus chronic) and for the type of white blood cell that is affected (e.g., myeloid versus lymphoid). Myeloid leukemias are also called myelogenous or myeloblastic leukemias. Lymphoid leukemias are also called lymphoblastic or lymphocytic leukemia. Lymphoid leukemia cells may collect in the lymph nodes, which can become swollen. Examples of leukemias include, but are not limited to: Acute myeloid leukemia (AML), Acute lymphoblastic leukemia (ALL), Chronic myeloid leukemia (CML), and Chronic lymphocytic leukemia (CLL).

Lymphomas are cancers that begin in cells of the immune system. For example, lymphomas can originate in bone marrow-derived cells that normally mature in the lymphatic system. There are two basic categories of lymphomas. One kind is Hodgkin lymphoma (HL), which is marked by the presence of a type of cell called the Reed-Stenberg cell. There are currently 6 recognized types of HL Examples of Hodgkin lymphomas include: nodular sclerosis classical Hodgkin lymphoma (CHL), mixed cellularity CHL, lymphocyte-depletion CHL, lymphocyte-rich CHL, and nodular lymphocyte predominant HL

The other category of lymphoma is non-Hodgkin lymphomas (NHL), which includes a large, diverse group of cancers of immune system cells. Non-Hodgkin lymphomas can be further divided into cancers that have an indolent (slow-growing) course and those that have an aggressive (fast-growing) course. There are currently 61 recognized types of NHL Examples of non-Hodgkin lymphomas include, but are not limited to: AIDS-related Lymphomas, anaplastic large-cell lymphoma, angioimmunoblastic lymphoma, blastic NK-cell lymphoma, Burkitt's lymphoma, Burkitt-like lymphoma (small non-cleaved cell lymphoma), chronic lymphocytic leukemia/small lymphocytic lymphoma, cutaneous T-Cell lymphoma, diffuse large B-Cell lymphoma, enteropathy-type T-Cell lymphoma, follicular lymphoma, hepatosplenic gamma-delta T-Cell lymphomas, T-Cell leukemias, lymphoblastic lymphoma, mantle cell lymphoma, marginal zone lymphoma, nasal T-Cell lymphoma, pediatric lymphoma, peripheral T-Cell lymphomas, primary central nervous system lymphoma, transformed lymphomas, treatment-related T-Cell lymphomas, and Waldenstrom's macroglobulinemia.

Brain cancers include any cancer of the brain tissues. Examples of brain cancers include, but are not limited to: gliomas (e.g., glioblastomas, astrocytomas, oligodendrogliomas, ependymomas, and the like), meningiomas, pituitary adenomas, vestibular schwannomas, primitive neuroectodermal tumors (medulloblastomas), etc.

The “pathology” of cancer includes all phenomena that compromise the well-being of the patient. This includes, without limitation, abnormal or uncontrollable cell growth, metastasis, interference with the normal functioning of neighboring cells, release of cytokines or other secretory products at abnormal levels, suppression or aggravation of inflammatory or immunological response, neoplasia, premalignancy, malignancy, invasion of surrounding or distant tissues or organs, such as lymph nodes, etc.

As used herein, the terms “cancer recurrence” and “tumor recurrence,” and grammatical variants thereof, refer to further growth of neoplastic or cancerous cells after diagnosis of cancer. Particularly, recurrence may occur when further cancerous cell growth occurs in the cancerous tissue. “Tumor spread,” similarly, occurs when the cells of a tumor disseminate into local or distant tissues and organs; therefore tumor spread encompasses tumor metastasis. “Tumor invasion” occurs when the tumor growth spread out locally to compromise the function of involved tissues by compression, destruction, or prevention of normal organ function.

As used herein, the term “metastasis” refers to the growth of a cancerous tumor in an organ or body part, which is not directly connected to the organ of the original cancerous tumor. Metastasis will be understood to include micrometastasis, which is the presence of an undetectable amount of cancerous cells in an organ or body part which is not directly connected to the organ of the original cancerous tumor. Metastasis can also be defined as several steps of a process, such as the departure of cancer cells from an original tumor site, and migration and/or invasion of cancer cells to other parts of the body.

In some embodiments, methods of inducing an immune response in an individual (in some cases referred to as methods of treating an individual) include: (a) contacting in vitro a dendritic cell (DC) from the individual with a target antigen and an antibody composition thereby producing a loaded DC; and (b) contacting a T cell of the individual with the loaded DC. The loaded DC presents antigens to the T cell to produce a contacted T cell, and the contacted T cell generates an immune response specific to the presented antigens.

Dendritic Cells.

A dendritic cell (DC) is a type of antigen-presenting cell of the mammalian immune system. The term “dendritic cell” as used herein refers to any member of a diverse population of morphologically similar cell types found in lymphoid or non-lymphoid tissues. These cells are characterized by their distinctive morphology and high levels of surface MHC-class II expression (Steinman, et al., Ann. Rev. Immunol. 9:271 (1991); hereby incorporated by reference for its description of such cells).

Dendritic cells are present in nearly all tissues such as the skin and the inner lining of the nose, lungs, liver, stomach, and intestines, as well as in bone marrow, blood, spleen, and lymph nodes. Once activated, DC migrate to the lymph nodes where they interact with T cells and B cells to initiate and shape the adaptive immune response. At certain development stages DC grow branched projections (the dendrites) that give the cells their name. Examples of dendritic cells include bone marrow-derived dendritic cells (BMDC), plasmacytoid dendritic cells, Langerhans cells, interdigitating cells, veiled cells, and dermal dendritic cells. In some cases, a DC expresses at least one marker selected from: CD11 (e.g., CD11a and/or CD11c), MHC-class II (for example, in the case of human, HLA-DR, HLA-DP and HLA-DQ), CD40, CD80 and CD86. In some cases, a DC is positive for HLA-DR and CD83, and negative for CD14. In general DC can be identified (e.g., the presence of DC can be verified) based on any or all of the markers: CD11c+; CD14-/low; CD80+; CD86++; MHC Class I++, MHC Class 11+++; CD40++; CD83+/−; CCR7+/−. In some cases, the DC is CD1b*/Gr1^{neg}/CD1c⁺/MHCII⁺/CD64^{dull}. In some cases, the DC is CD11b^{neg}/CD11c^{hi}/MHCII⁺.

In some cases, the dendritic cell expresses a specific Ig Fc receptor. For example, a dendritic cell can express an Fc-γ receptor which recognizes IgG antibodies, or antibodies that contain an Fc region of an IgG. As another example, the dendritic cell can express an Fc-α receptor which recognizes IgA antibodies, or antibodies that contain an Fc region of an IgA. As yet another example, the dendritic cell can express an Fc-E receptor which recognizes IgE antibodies, or antibodies that contain an Fc region of an IgE. In some cases, dendritic cells expressing a specific Fc receptor are obtained and loaded with an appropriate bridging molecule (e.g., allogeneic Ig of a class recognized by the dendritic cell Fc receptor).

In some embodiments, subject methods include a step of obtaining or isolating a DC (e.g., isolating enriched populations of DC). Techniques for the isolation, generation, and culture of DC will be known to one of ordinary skill in the art and any convenient technique can be used. In some cases, the DC are autologous to the individual who is being treated (i.e., are cells isolated from the individual or are cells derived from cells of the individual).

In some cases, CD34(+) progenitors (e.g., bone marrow (BM) progenitor cells) are used as a source for generating DCs (e.g., CD34⁺ cells can be enriched using, for example, antibody-bound magnetic beads), which are then referred to as bone marrow (BM) derived dendritic cells (BMDC). For example, BMDCs can be generated by culturing nonadherent cells (CD34+cells) in the presence of a cytokine that functions as a white blood cell growth factor (e.g., granulocyte-macrophage-colony stimulating factor (GM-CSF), e.g., 50 ng/ml) and a cytokine (e.g., intereukin 4 (IL-4), e.g., 20 ng/ml). In some cases, the CD34+cells are cultured in the presence of GM-CSF and/or IL-4 for a period of time in a range of from 4 days to 18 days (e.g., 5 days to 17 days, 7 days to 16 days, 8 days to 13 days, 9 days to 12 days, 6 days to 15 days, 8 days to 15 days, 10 days to 15 days, 12 days to 15 days, 13 days to 15 days, 5 days to 14 days, 5 days to 12 days, 5 days to 10 days, 5 days to 9 days, 6 days to 8 days, 6 days, 7 days, 8 days, 9 days, 10 days, 12 days, or 14 days). When CD34+cells are cultured in the presence of GM-CSF and/or IL-4, the GM-CSF can be at a concentration in a range of from 35 ng/ml to 65 ng/ml (35 ng/ml to 65 ng/ml, 40 ng/ml to 60 ng/ml, 45 ng/ml to 50 ng/ml, or 50 ng/ml) and the IL-4 can be at a concentration in a range of from 5 ng/ml to 35 ng/ml (10 ng/ml to 30 ng/ml, 15 ng/ml to 25 ng/ml, 17.5 ng/ml to 22.5 ng/ml, or 20 ng/ml). As an illustrative example, bones can flushed with a saline solution (e.g., phosphate buffered saline (PBS)) and mononuclear cells can be separated from the bone marrow on Ficoll gradients. CD34+cells can then be isolated/enriched (e.g., using antibody-conjugated magnetic beads) and then cultured in the presence of GM-CSF and IL-4 (as described above). In some cases (e.g, when the cells are mouse cells), DCs can be derived by culturing the cells in GM-CSF. In some cases (e.g, when the cells are human cells), DCs can be derived by culturing the cells in GM-CSF and IL-4.

In some cases, monocytes are used as a source for generating DCs (sometimes referred to as blood derived DCs, blood Mo-DCs, monocyte DCs, and the like). For example, DCs can be generated by culturing adherent cells (monocytes, e.g., bone marrow monocytes, blood monocytes, etc.)(e.g., CD14+blood monocytes) in the presence of GM-CSF (e.g., at a concentration in a range as described above for BMDC) and/or IL-4 (e.g., at a concentration in a range as described above for BMDC) for a period of time in a range of from 3 days to 9 days (e.g., 4 days to 8 days, 5 days to 7 days, 3 days to 6 days, 4 days to 5 days, 6 days to 8 days, or 7 days). For example, in some cases, mononuclear cells are isolated from blood and enriched for CD11b+cells (e.g., using magnetic beads). The cells can be sorted for “inflammatory monocytes” (FSC^{lo}/SSC^{lo}/Gr1^{hi}/CD115^{hi}) and/or “patrolling monocytes” (FSC^{lo}/SSC^{lo}/Gr1^{neg}/CD115^{hi}). DCs can then be generated from various types of monocytes by culturing the monocytes in the presence of GM-CSF (e.g., for a period of time in a range of from 3 days to 6 days (e.g., 4 days to 5 days)). In some cases (e.g, when the cells are mouse cells), DCs are derived by culturing the cells in GM-CSF. In some cases (e.g, when the cells are human cells), DCs are derived by culturing the cells in GM-CSF and IL-4. To obtain DC from spleen (a splenic DC), splenocytes can be enriched (e.g., using antibody-coupled magnetic beads) for CD1c⁺ cells and CD11c^{hi}/MHCII^{hi} cells can be sorted/enriched using flow cytometry (e.g., FACS).

In some cases, DC are tumor associated DC (TADC). TADC can be obtained by any convenient method. For example, to obtain DC from tumors (tumor associated DC, TADC), tumors can be digested (e.g., using collagenase and nuclease) and CD11c+cells can be enriched (e.g., using antibody-conjugated magnetic beads), and Gr1^{neg}/CD11c^{hi}/MHCII^{hi} cells can be sorted/enriched using flow cytometry (e.g., FACS).

Isolated and/or derived DCs (e.g., as described above) can be activated using various factors including, but not limited to TNFα (e.g., 50 ng/ml) and a CD40 ligand (e.g., CD40L) (e.g., 500 ng/ml) (described in further detail below).

For more information regarding dendritic cells and methods of isolating, generating, and/or culturing DC, see: Vassalli, J Transplant. 2013; 2013: 761429: “Dendritic Cell-Based Approaches for Therapeutic Immune Regulation in Solid-Organ Transplantation”; Syme et al., Stem Cells. 2005; 23(1):74-81: “Comparison of CD34 and monocyte-derived dendritic cells from mobilized peripheral blood from cancer patients”; Banchereau et al., Annu Rev Immunol. 2000; 18:767-811: “Immunobiology of dendritic cells”; and U.S. patent application numbers 20130330822; 20130273654; 20130130380; 20120251561; and 20120244620; all of which are hereby incorporated by reference in their entirety.

In some cases (e.g., when the method includes administering to the individual an antibody composition) an endogenous DC (a DC present in the individual) is contacted in vivo with the administered antibody composition. Thus, the method can be considered an in vivo method of treating an individual having cancer. For example, an antibody composition and/or a dendritic cell stimulatory composition can be administered to (e.g., injected into) an individual (e.g., injected into or near a tumor, into or near a site of tumor resection, and the like) and endogenous DCs are thereby contacted with the antibody composition and/or the dendritic cell stimulatory composition. The loaded DC can then contact endogenous T cells in vivo (additional details are provided below with respect to in vivo methods; see the section entitled “Contacting a T cell with a loaded DC”). In some cases (e.g., where the DC is a BMDC), a dendritic cell stimulatory composition is not used.

Macrophages.

A macrophage is a type of antigen-presenting cell of the mammalian immune system. The term “macrophage” as used herein refers to any member of a diverse population of morphologically similar cell types found in lymphoid or non-lymphoid tissues. These cells are characterized by their distinctive morphology and high levels of surface MHC-class II expression. A macrophage is a monocyte-derived phagocyte which is not a dendritic cell or a cell that derives from tissue macrophages by local proliferation. In the body these cells are tissue specific and refer to e. g. Kupffer cells in the liver, alveolar macrophages in the lung, microglia cells in the brain, osteoclasts in the bone etc. The skilled person is aware how to identify macrophage cells, how to isolate macrophage cells from the body of a human or animal, and how to characterize macrophage cells with respect to their subclass and subpopulation (Kruisbeek, 2001; Davies and Gordon 2005 a and b; Zhang et al., 2008; Mosser and Zhang, 2008; Weischenfeldt and Porse, 2008; Ray and Dittel, 2010; Martinez et al., 2008; Jenkins et al., 2011).

Macrophages can be activated by different mechanisms into different subclasses, including, but not limited to M1, M2, M2a, M2b, and M2c subclasses. Whereas the term M1 is used to describe classically activated macrophages that arise due to injury or bacterial infection and IFN-γ activation, M2 is a generic term for numerous forms of macrophages activated differently than M1. The M2 classification has further been divided into subpopulations (Mantovani et al., 2004). The most representative form is M2a macrophages, which commonly occur in helminth infections by exposure to worm induced Th2 cytokines IL-4 and IL-13. M2a macrophages were, among others, shown to be essentially involved in protecting the host from re-infection (Anthony et al., 2006) or in contributing to wound healing and tissue remodeling (Gordon, 2003). Another subpopulation is M2b macrophages that produce high levels of IL-10 and low levels of IL-12 but are not per se anti-inflammatory (Anderson and Mosser, 2002; Edwards et al., 2006). M2b macrophages are elicited by immune complexes that bind to Fc-γ receptors in combination with TLR ligands. Finally, M2c macrophages represent a subtype elicited by IL-10, TGF-β or glucocorticoids (Martinez et al., 2008).

Thus, “M2a macrophages” refers to a macrophage cell that has been exposed to a milieu under Th2 conditions (e g. exposure to Th2 cytokines IL-4 and IL-13) and exhibits a specific phenotype by higher expression of the gene Ym1 and/or the gene CD206 and/or the gene RELM-α and/or the gene Arginase-1. Similarly, “M2b macrophages” refers to a macrophage cell that has been exposed to a milieu of immune complexes in combination with TLR or TNF-alpha stimulation. Said cell is characterized through higher expression of the gene SPHK-1 and/or the gene LIGHT and/or the gene IL-10.

In some cases, the present application refers to a macrophage cell “derived from the body of a patient”. This is meant to designate that either macrophages are obtained from the body of said patient, or macrophage precursor cells are obtained from the body of said patient and subsequently differentiated into macrophage cells in vitro as described in Wahl et al. 2006; Davis and Gordon 2005; Smythies et al., 2006; Zhang et al., 2008; Mosser and Zhang, 2008.

B-Cells.

A B-cell is a type of antigen-presenting cell of the mammalian immune system. The term “B-cell” as used herein refers to B-cells from any stage of development (e.g., B-stem cells, progenitor B-cells, differentiated B-cells, plasma cells) and from any source including, but not limited to peripheral blood, a region at, in, or near a tumor, lymph nodes, bone marrow, umbilical chord blood, or spleen cells.

B-cell precursors reside in the bone marrow where immature B-cells are produced. B-cell development occurs through several stages, each stage representing a change in the genome content at the antibody loci. In the genomic heavy chain variable region there are three segments, V, D, and J, which recombine randomly, in a process called VDJ rearrangement to produce a unique variable region in the immunoglobulin of each B-cell. Similar rearrangements occur for the light chain variable region except that there are only two segments involved, V and J. After complete rearrangement, the B-cell reaches the IgM+immature stage in the bone marrow. These immature B-cells present a membrane bound IgM, i.e., BCR, on their surface and migrate to the spleen, where they are called transitional B cells. Some of these cells differentiate into mature B lymphocytes. Mature B-cells expressing the BCR on their surface circulate the blood and lymphatic system performing the role of immune surveillance. They do not produce soluble antibodies until they become fully activated. Each B-cell has a unique receptor protein that will bind to one particular antigen. Once a B-cell encounters its antigen and receives an additional signal from a T helper cell, it can further differentiate into either a plasma B-cell expressing and secreting soluble antibodies or a memory B-cell.

In the context of the present disclosure, the term “B-cell” refers to any B lymphocyte which presents a fully rearranged, i.e., a mature, BCR on its surface. For example, a B-cell in the context of the present invention may be an immature or a mature B-cell. In some cases, the B-cell is a naive B-cell, i.e., a B-cell that has not been exposed to the antigen specifically recognized by the BCR on the surface of said B-cell. In some embodiments, the B-cells are CD19+ B-cells, i.e., express CD19 on their surface. In some cases, the B-cells in the context of the present invention are CD19+ B-cells and express a fully rearranged BCR on their surface. The B-cells may also be CD20+ or CD21+ B-cells. In some cases, the CD20+ or CD21+ B-cells carry a BCR on their surface. In some embodiments, the B-cells are memory B-cells, such as IgG+memory B cells.

Treatments that Activate Antigen Presenting Cells (APC). e.g., Dendritic Cells (DC).

In some embodiments, a subject method includes administering to the individual a treatment that activates APC, e.g., DC, of the individual. Such a step can be performed in vivo or in vitro, and can be performed prior to, after, or simultaneous with a step of administering an antibody composition. Any convenient treatment that activates APC, e.g., DC, can be performed. For example, in some cases, a treatment that activates (stimulates) APC, e.g., DC, can include any form of cancer therapy that activates endogenous APC, e.g., DC, (e.g., local irradiation of an individual, e.g., 200-4,000 rads of ionizing radiation; chemotherapy; and the like). In some cases, a treatment that activates an APC does not include local irradiation. In some cases, a treatment that activates an APC, e.g., DC, (e.g., activates dendritic cells) includes contacting an APC, e.g., DC, (e.g., an endogenous DC (i.e., a DC in vivo, e.g., a TADC in vivo), a DC that is not a BMDC, a DC that is a BMDC, a TADC, a macrophage, a B-cell, etc.) with an APC stimulatory composition, e.g., a dendritic cell stimulatory composition. In some cases, APC, e.g., DC, are activated in vivo. In some cases, APC, e.g., DC, are activated ex vivo (e.g., a DC, e.g., a TADC, can be isolated from an individual, and the TADC can then be activated, e.g., contacted with a dendritic cell stimulatory composition).

Dendritic Cell Stimulatory Composition.

In some cases, a treatment that activates a dendritic cell (e.g., activates dendritic cells) includes contacting a DC (e.g., an endogenous DC, a DC that is not a BMDC, a DC that is a BMDC, a TADC, etc.) with a dendritic cell stimulatory composition. As used herein, a “dendritic cell stimulatory composition” includes at least one dendritic cell stimulatory agent.

Dendritic cell stimulatory agents are agents that activate DC, and/or stimulate the uptake of antigen (e.g., stimulate the uptake, e.g., phagocytosis, of a tumor cell), and/or stimulate the maturation of DC, and/or stimulate the presentation of antigen to T cells. Suitable dendritic cell stimulatory agents include, but are not limited to: CD40 agonists, proinflammatory cytokines, Toll-like receptor agonists (e.g., a CpG ODN, polyinosinic:polycytidylic acid (“poly I:C”, a TLR-3 agonist), etc.), indoleamine 2,3-dioxygenase (IDO) inhibitors, checkpoint molecule neutralizing compounds (e.g., antibodies that neutralize checkpoint molecules, e.g., an anti-CTLA-4 antibody, e.g., Ipilimumab), NFkB activators (e.g., phorbol esters), compounds that open calcium channels (e.g., ionomycin), T cell-related co-stimulatory molecules (e.g., CD27, CD28, 4-BBL, and the like), and any combination thereof.

For example, in some cases, a subject dendritic cell stimulatory composition includes a CD40 agonist (e.g., CD40L and/or an agonistic anti-CD40 antibody) and a proinflammatory cytokine (e.g., TNFα, IL-1α, IL-1β, IL-19, interferon gamma (IFNγ), and the like). In some cases, a subject dendritic cell stimulatory composition includes a CD40 agonist (e.g., CD40L and/or an agonistic anti-CD40 antibody), a proinflammatory cytokine (e.g., TNFα, IL-1α, IL-1P, IL-19, interferon gamma (IFNγ), and the like), and a Toll-like receptor agonist (e.g., a CpG ODN, polyinosinic:polycytidylic acid (“poly I:C”, a TLR-3 agonist), etc.). In some cases, a subject dendritic cell stimulatory composition includes a Toll-like receptor agonist (e.g., a CpG ODN, polyinosinic:polycytidylic acid (“poly I:C”, a TLR-3 agonist), etc.). In some cases, a subject dendritic cell stimulatory composition includes an IDO inhibitor. In some cases, a subject dendritic cell stimulatory composition includes an antibody that neutralizes checkpoint molecules (e.g., an anti-CTLA-4 antibody, e.g., Ipilimumab). In some cases a subject dendritic cell stimulatory composition includes a T cell-related co-stimulatory molecule (e.g., CD27, CD28, 4-BBL, and the like). In some cases a subject dendritic cell stimulatory composition includes a T cell-related co-stimulatory molecule (e.g., CD27, CD28, 4-BBL, and the like) and a proinflammatory cytokine (e.g., TNFα, IL-1α, IL-1P, IL-19, interferon gamma (IFNγ), and the like). In some cases a subject dendritic cell stimulatory composition includes a T cell-related co-stimulatory molecule (e.g., CD27, CD28, 4-BBL, and the like), a proinflammatory cytokine (e.g., TNFα, IL-1α, IL-1p, IL-19, interferon gamma (IFNγ), and the like), and a Toll-like receptor agonist (e.g., a CpG ODN, polyinosinic:polycytidylic acid (“poly I:C”, a TLR-3 agonist), etc.).

B-Cell Stimulatory Composition.

In some cases, a treatment that activates a B-cell (e.g., activates B-cells) includes contacting a B-cell with a B-cell stimulatory composition. As used herein, a “B-cell stimulatory composition” includes at least one B-cell stimulatory agent B-cell stimulatory agents are agents that activate B-cells, and/or stimulate the uptake of antigen (e.g., stimulate the uptake, e.g., phagocytosis, of a tumor cell), and/or stimulate the maturation of a B-cell, and/or stimulate the presentation of antigen to T cells. Suitable B-cell stimulatory agents include, but are not limited to a Toll-like receptor (TLR) agonists; (ii) CD40 agonists; (iii) a CD40 agonist and a proinflammatory cytokine; (iv) an antigen that binds the B-cell receptor; (v) an anti-idiotype antibody; (vi) an agent that cross-links surface immunoglobulin; and any combination thereof.

Macrophage Stimulatory Composition.

In some cases, a treatment that activates a macrophage (e.g., activates macrophages) includes contacting a macrophage with a macrophage stimulatory composition. As used herein, a “macrophage stimulatory composition” includes at least one macrophage stimulatory agent.

Macrophage stimulatory agents are agents that activate macrophages, and/or stimulate the uptake of antigen (e.g., stimulate the uptake, e.g., phagocytosis, of a tumor cell), and/or stimulate the maturation of a macrophage, and/or stimulate the presentation of antigen to T cells. Suitable macrophage stimulatory agents include, but are not limited to a Toll-like receptor (TLR) agonists; (ii) a macrophage activating cytokine; (iii) a glucocorticoid receptor agonist; and any combination thereof.

Any convenient CD40 agonist can be used. Examples of suitable CD40 agonists include, but are not limited to: anagonistic anti-CD40 antibody, CD40 ligand (CD40L, also known as CD40LG), and the like. Any convenient agonistic anti-CD40 antibody can be used and agonistic anti-CD40 antibodies are known in the art. Any convenient CD40L (or functional fragment thereof) may be used. For example, human CD40L is a polypeptide having the protein sequence:

(SEQ ID NO: 1)
MIETYNQTSPRSAATGLPISMKIFMYLLTVFLITQMIGSALFAVYLHRR
LDKIEDERNLHEDFVFMKTIQRCNTGERSLSLLNCEEIKSQFEGFVKDI
MLNKEETKKENSFEMQKGDQNPQIAAHVISEASSKTTSVLQWAEKGYYT
MSNNLVTLENGKQLTVKRQGLYYIYAQVTFCSNREASSQAPFIASLCLK
SPGRFERILLRAANTHSSAKPCGQQSIHLGGVFELQPGASVFVNVTDPS
QVSHGTGFTSFGLLKL,

which is encoded by the corresponding mRNA of the following cDNA sequence (the open reading frame is underlined):

(SEQ ID NO: 2)
ACTTTGACAGTCTTCTCATGCTGCCTCTGCCACCTTCTCTGCCAGAAGA
TACCATTTCAACTTTAACACAGCATGATCGAAACATACAACCAAACTTC
TCCCCGATCTGCGGCCACTGGACTGCCCATCAGCATGAAAATTTTTATG
TATTTACTTACTGTTTTTCTTATCACCCAGATGATTGGGTCAGCACTTT
TTGCTGTGTATCTTCATAGAAGGTTGGACAAGATAGAAGATGAAAGGAA
TCTTCATGAAGATTTTGTATTCATGAAAACGATACAGAGATGCAACACA
GGAGAAAGATCCTTATCCTTACTGAACTGTGAGGAGATTAAAAGCCAGT
TTGAAGGCTTTGTGAAGGATATAATGTTAAACAAAGAGGAGACGAAGAA
AGAAAACAGCTTTGAAATGCAAAAAGGTGATCAGAATCCTCAAATTGCG
GCACATGTCATAAGTGAGGCCAGCAGTAAAACAACATCTGTGTTACAGT
GGGCTGAAAAAGGATACTACACCATGAGCAACAACTTGGTAACCCTGGA
AAATGGGAAACAGCTGACCGTTAAAAGACAAGGACTCTATTATATCTAT
GCCCAAGTCACCTTCTGTTCCAATCGGGAAGCTTCGAGTCAAGCTCCAT
TTATAGCCAGCCTCTGCCTAAAGTCCCCCGGTAGATTCGAGAGAATCTT
ACTCAGAGCTGCAAATACCCACAGTTCCGCCAAACCTTGCGGGCAACAA
TCCATTCACTTGGGAGGAGTATTTGAATTGCAACCAGGTGCTTCGGTGT
TTGTCAATGTGACTGATCCAAGCCAAGTGAGCCATGGCACTGGCTTCAC
GTCCTTTGGCTTACTCAAACTCTGAACAGTGTCACCTTGCAGGCTGTGG
TGGAGCTGACGCTGGGAGTCTTCATAATACAGCACAGCGGTTAAGCCCA
CCCCCTGTTAACTGCCTATTTATAACCCTAGGATCCTCCTTATGGAGAA
CTATTTATTATACACTCCAAGGCATGTAGAACTGTAATAAGTGAATTAC
AGGTCACATGAAACCAAAACGGGCCCTGCTCCATAAGAGCTTATATATC
TGAAGCAGCAACCCCACTGATGCAGACATCCAGAGAGTCCTATGAAAAG
ACAAGGCCATTATGCACAGGTTGAATTCTGAGTAAACAGCAGATAACTT
GCCAAGTTCAGTTTTGTTTCTTTGCGTGCAGTGTCTTTCCATGGATAAT
GCATTTGATTTATCAGTGAAGATGCAGAAGGGAAATGGGGAGCCTCAGC
TCACATTCAGTTATGGTTGACTCTGGGTTCCTATGGCCTTGTTGGAGGG
GGCCAGGCTCTAGAACGTCTAACACAGTGGAGAACCGAAACCCCCCCCC
CCCCCCCGCCACCCTCTCGGACAGTTATTCATTCTCTTTCAATCTCTCT
CTCTCCATCTCTCTCTTTCAGTCTCTCTCTCAACCTCTTTCTTCCAATC
TCTCTTTCTCAATCTCTCTGTTTCCCTTTGTCAGTCTCTTCCCTCCCCC
AGTCTCTCTTCTCAATCCCCCTTTCTAACACACACACACACACACACAC
ACACACACACACACACACACACACACACACAGAGTCAGGCCGTTGCTAG
TCAGTTCTCTTCTTTCCACCCTGTCCCTATCTCTACCACTATAGATGAG
GGTGAGGAGTAGGGAGTGCAGCCCTGAGCCTGCCCACTCCTCATTACGA
AATGACTGTATTTAAAGGAAATCTATTGTATCTACCTGCAGTCTCCATT
GTTTCCAGAGTGAACTTGTAATTATCTTGTTATTTATTTTTTGAATAAT
AAAGACCTCTTAACATTAA.

A suitable CD40L can also be a functional fragment of CD40L (i.e., the CD40L need not be the full length polypeptide). The membrane-anchored CD4-g and is expressed on CD4+T lymphocytes. The soluble form of CD40L is a protein comprising the entire TNF homologous region of CD40L and is generated in vivo by an intracellular proteolytic processing of the full length CD40L. For example, recombinant murine CD40L can be a soluble 16.4 kDa protein containing 149 amino acid residues having the receptor binding TNF-like domain of CD40L:

(SEQ ID NO: 3)
MQRGDEDPQIAAHVVSEANSNAASVLQWAKKGYYTMKSNLVMLENGKQL
TVKREGLYYVYTQVTFCSNREPSSQRPFIVGLWLKPSSGSERILLKAAN
THSSSQLCEQQSVHLGGVFELQAGASVFVNVTEASQVIHRVGFSSFGLL
KL.

As another example, recombinant human soluble CD40 ligand (CD4L) can be a 16.3 kDa protein containing 149 amino acid residues having the receptor binding TNF-like domain of CD40L:

(SEQ ID NO: 4)
MQKGDQNPQIAAHVISEASSKTTSVLQWAEKGYYTMSNNLVTLENGKQL
TVKRQGLYYIYAQVTFCSNREASSQAPFIASLWLKSPGRFERILLRAAN
THSSAKPCGQQSIHLGGVFELQPGASVFVNVTDPSQVSHGTGFTSFGLL
KL.

A suitable CD40L (including a functional fragment) can also be provide as a nucleic acid (e.g., DNA and/or mRNA) that encodes a CD40L polypeptide (e.g., full length, functional fragment, etc.).

When CD40L is used, it can be used at any convenient concentration to achieve loading of the APC, e.g., DC. For example, in some cases, CD40L is used at a concentration in a range of from 350 ng/ml to 650 ng/ml (e.g., from 400 ng/ml to 600 ng/ml, from 425 ng/ml to 575 ng/ml, from 450 ng/ml to 550 ng/ml, from 475 ng/ml to 525 ng/ml, or 500 ng/ml).

For more information about CD40 agonists and non-limiting examples of CD40 agonists, refer to: Khong et al., Int Rev Immunol. 2012 August; 31(4):246-66; Khong et al., J Immunother. 2013 September; 36(7):365-72; Rycyzyn et al., Hybridoma (Larchmt). 2008 February; 27(1):25-30; Khalil et al., Update Cancer Ther. 2007 Jun. 1; 2(2):61-85; and U.S. patent applications 20130024956; 20120225014; and 20100098694; all of which are hereby incorporated by reference in their entirety.

Any convenient proinflammatory cytokine, or inducer of proinflammatory cytokines, can be used. Examples of suitable proinflammatory cytokines include, but are not limited to: tumor necrosis factor (TNF, also known as tumor necrosis factor alpha (TNFα)); interleukin (IL) 1 (IL-I) (e.g., IL-la, IL-10); and IL-19.

For example, human tumor necrosis factor (TNF, TNFα) is a polypeptide having the protein sequence:

(SEQ ID NO: 5)
MSTESMIRDVELAEEALPKKTGGPQGSRRCLFLSLFSFLIVAGATTLFC
LLHFGVIGPQREEFPRDLSLISPLAQAVRSSSRTPSDKPVAHVVANPQA
EGQLQWLNRRANALLANGVELRDNQLVVPSEGLYLIYSQVLFKGQGCPS
THVLLTHTISRIAVSYQTKVNLLSAIKSPCQRETPEGAEAKPWYEPIYL
GGVFQLEKGDRLSAEINRPDYLDFAESGQVYFGIIAL,

which is encoded by the corresponding mRNA of the following cDNA sequence (the open reading frame is underlined):

(SEQ ID NO. 6)
CAGACGCTCCCTCAGCAAGGACAGCAGAGGACCAGCTAAGAGGGAGAGA
AGCAACTACAGACCCCCCCTGAAAACAACCCTCAGACGCCACATCCCCT
GACAAGCTGCCAGGCAGGTTCTCTTCCTCTCACATACTGACCCACGGCT
CCACCCTCTCTCCCCTGGAAAGGACACCATGAGCACTGAAAGCATGATC
CGGGACGTGGAGCTGGCCGAGGAGGCGCTCCCCAAGAAGACAGGGGGGC
CCCAGGGCTCCAGGCGGTGCTTGTTCCTCAGCCTCTTCTCCTTCCTGAT
CGTGGCAGGCGCCACCACGCTCTTCTGCCTGCTGCACTTTGGAGTGATC
GGCCCCCAGAGGGAAGAGTTCCCCAGGGACCTCTCTCTAATCAGCCCTC
TGGCCCAGGCAGTCAGATCATCTTCTCGAACCCCGAGTGACAAGCCTGT
AGCCCATGTTGTAGCAAACCCTCAAGCTGAGGGGCAGCTCCAGTGGCTG
AACCGCCGGGCCAATGCCCTCCTGGCCAATGGCGTGGAGCTGAGAGATA
ACCAGCTGGTGGTGCCATCAGAGGGCCTGTACCTCATCTACTCCCAGGT
CCTCTTCAAGGGCCAAGGCTGCCCCTCCACCCATGTGCTCCTCACCCAC
ACCATCAGCCGCATCGCCGTCTCCTACCAGACCAAGGTCAACCTCCTCT
CTGCCATCAAGAGCCCCTGCCAGAGGGAGACCCCAGAGGGGGCTGAGGC
CAAGCCCTGGTATGAGCCCATCTATCTGGGAGGGGTCTTCCAGCTGGAG
AAGGGTGACCGACTCAGCGCTGAGATCAATCGGCCCGACTATCTCGACT
TTGCCGAGTCTGGGCAGGTCTACTTTGGGATCATTGCCCTGTGAGGAGG
ACGAACATCCAACCTTCCCAAACGCCTCCCCTGCCCCAATCCCTTTATT
ACCCCCTCCTTCAGACACCCTCAACCTCTTCTGGCTCAAAAAGAGAATT
GGGGGCTTAGGGTCGGAACCCAAGCTTAGAACTTTAAGCAACAAGACCA
CCACTTCGAAACCTGGGATTCAGGAATGTGTGGCCTGCACAGTGAAGTG
CTGGCAACCACTAAGAATTCAAACTGGGGCCTCCAGAACTCACTGGGGC
CTACAGCTTTGATCCCTGACATCTGGAATCTGGAGACCAGGGAGCCTTT
GGTTCTGGCCAGAATGCTGCAGGACTTGAGAAGACCTCACCTAGAAATT
GACACAAGTGGACCTTAGGCCTTCCTCTCTCCAGATGTTTCCAGACTTC
CTTGAGACACGGAGCCCAGCCCTCCCCATGGAGCCAGCTCCCTCTATTT
ATGTTTGCACTTGTGATTATTTATTATTTATTTATTATTTATTTATTTA
CAGATGAATGTATTTATTTGGGAGACCGGGGTATCCTGGGGGACCCAAT
GTAGGAGCTGCCTTGGCTCAGACATGTTTTCCGTGAAAACGGAGCTGAA
CAATAGGCTGTTCCCATGTAGCCCCCTGGCCTCTGTGCCTTCTTTTGAT
TATGTTTTTTAAAATATTTATCTGATTAAGTTGTCTAAACAATGCTGAT
TTGGTGACCAACTGTCACTCATTGCTGAGCCTCTGCTCCCCAGGGGAGT
TGTGTCTGTAATCGCCCTACTATTCAGTGGCGAGAAATAAAGTTTGCTT
AGAAAAGAAAAAAAAAAAAA.

When TNFα is used, it can be used at any convenient concentration to achieve loading of the APC, e.g., DC. For example, in some cases, TNFα is used at a concentration in a range of from 20 ng/ml to 80 ng/ml (e.g., from 25 ng/ml to 75 ng/ml, from 30 ng/ml to 70 ng/ml, from 35 ng/ml to 65 ng/ml, from 40 ng/ml to 60 ng/ml, from 45 ng/ml to 55 ng/ml, from 47.5 ng/ml to 52.5 ng/ml, or 50 ng/ml).

As another example, human IL-1a is a polypeptide having the protein sequence:

(SEQ ID NO: 7)
MAKVPDMFEDLKNCYSENEEDSSSIDHLSLNQKSFYHVSYGPLHEGCMD
QSVSLSISETSKTSKLTFKESMVVVATNGKVLKKRRLSLSQSITDDDLE
AIANDSEEEIIKPRSAPFSFLSNVKYNFMRIIKYEFILNDALNQSIIRA
NDQYLTAAALHNLDEAVKFDMGAYKSSKDDAKITVILRISKTQLYVTAQ
DEDQPVLLKEMPEIPKTITGSETNLLFFWETHGTKNYFTSVAHPNLFIA
TKQDYWVCLAGGPPSITDFQILENQA,

which is encoded by the corresponding mRNA of the following cDNA sequence (the open reading frame is underlined):

(SEQ ID NO: 8)
ACCAGGCAACACCATTGAAGGCTCATATGTAAAAATCCATGCCTTCCTT
TCTCCCAATCTCCATTCCCAAACTTAGCCACTGGCTTCTGGCTGAGGCC
TTACGCATACCTCCCGGGGCTTGCACACACCTTCTTCTACAGAAGACAC
ACCTTGGGCATATCCTACAGAAGACCAGGCTTCTCTCTGGTCCTTGGTA
GAGGGCTACTTTACTGTAACAGGGCCAGGGTGGAGAGTTCTCTCCTGAA
GCTCCATCCCCTCTATAGGAAATGTGTTGACAATATTCAGAAGAGTAAG
AGGATCAAGACTTCTTTGTGCTCAAATACCACTGTTCTCTTCTCTACCC
TGCCCTAACCAGGAGCTTGTCACCCCAAACTCTGAGGTGATTTATGCCT
TAATCAAGCAAACTTCCCTCTTCAGAAAAGATGGCTCATTTTCCCTCAA
AAGTTGCCAGGAGCTGCCAAGTATTCTGCCAATTCACCCTGGAGCACAA
TCAACAAATTCAGCCAGAACACAACTACAGCTACTATTAGAACTATTAT
TATTAATAAATTCCTCTCCAAATCTAGCCCCTTGACTTCGGATTTCACG
ATTTCTCCCTTCCTCCTAGAAACTTGATAAGTTTCCCGCGCTTCCCTTT
TTCTAAGACTACATGTTTGTCATCTTATAAAGCAAAGGGGTGAATAAAT
GAACCAAATCAATAACTTCTGGAATATCTGCAAACAACAATAATATCAG
CTATGCCATCTTTCACTATTTTAGCCAGTATCGAGTTGAATGAACATAG
AAAAATACAAAACTGAATTCTTCCCTGTAAATTCCCCGTTTTGACGACG
CACTTGTAGCCACGTAGCCACGCCTACTTAAGACAATTACAAAAGGCGA
AGAAGACTGACTCAGGCTTAAGCTGCCAGCCAGAGAGGGAGTCATTTCA
TTGGCGTTTGAGTCAGCAAAGAAGTCAAGATGGCCAAAGTTCCAGACAT
GTTTGAAGACCTGAAGAACTGTTACAGTGAAAATGAAGAAGACAGTTCC
TCCATTGATCATCTGTCTCTGAATCAGAAATCCTTCTATCATGTAAGCT
ATGGCCCACTCCATGAAGGCTGCATGGATCAATCTGTGTCTCTGAGTAT
CTCTGAAACCTCTAAAACATCCAAGCTTACCTTCAAGGAGAGCATGGTG
GTAGTAGCAACCAACGGGAAGGTTCTGAAGAAGAGACGGTTGAGTTTAA
GCCAATCCATCACTGATGATGACCTGGAGGCCATCGCCAATGACTCAGA
GGAAGAAATCATCAAGCCTAGGTCAGCACCTTTTAGCTTCCTGAGCAAT
GTGAAATACAACTTTATGAGGATCATCAAATACGAATTCATCCTGAATG
ACGCCCTCAATCAAAGTATAATTCGAGCCAATGATCAGTACCTCACGGC
TGCTGCATTACATAATCTGGATGAAGCAGTGAAATTTGACATGGGTGCT
TATAAGTCATCAAAGGATGATGCTAAAATTACCGTGATTCTAAGAATCT
CAAAAACTCAATTGTATGTGACTGCCCAAGATGAAGACCAACCAGTGCT
GCTGAAGGAGATGCCTGAGATACCCAAAACCATCACAGGTAGTGAGACC
AACCTCCTCTTCTTCTGGGAAACTCACGGCACTAAGAACTATTTCACAT
CAGTTGCCCATCCAAACTTGTTTATTGCCACAAAGCAAGACTACTGGGT
GTGCTTGGCAGGGGGGCCACCCTCTATCACTGACTTTCAGATACTGGAA
AACCAGGCGTAGGTCTGGAGTCTCACTTGTCTCACTTGTGCAGTGTTGA
CAGTTCATATGTACCATGTACATGAAGAAGCTAAATCCTTTACTGTTAG
TCATTTGCTGAGCATGTACTGAGCCTTGTAATTCTAAATGAATGTTTAC
ACTCTTTGTAAGAGTGGAACCAACACTAACATATAATGTTGTTATTTAA
AGAACACCCTATATTTTGCATAGTACCAATCATTTTAATTATTATTCTT
CATAACAATTTTAGGAGGACCAGAGCTACTGACTATGGCTACCAAAAAG
ACTCTACCCATATTACAGATGGGCAAATTAAGGCATAAGAAAACTAAGA
AATATGCACAATAGCAGTTGAAACAAGAAGCCACAGACCTAGGATTTCA
TGATTTCATTTCAACTGTTTGCCTTCTACTTTTAAGTTGCTGATGAACT
CTTAATCAAATAGCATAAGTTTCTGGGACCTCAGTTTTATCATTTTCAA
AATGGAGGGAATAATACCTAAGCCTTCCTGCCGCAACAGTTTTTTATGC
TAATCAGGGAGGTCATTTTGGTAAAATACTTCTTGAAGCCGAGCCTCAA
GATGAAGGCAAAGCACGAAATGTTATTTTTTAATTATTATTTATATATG
TATTTATAAATATATTTAAGATAATTATAATATACTATATTTATGGGAA
CCCCTTCATCCTCTGAGTGTGACCAGGCATCCTCCACAATAGCAGACAG
TGTTTTCTGGGATAAGTAAGTTTGATTTCATTAATACAGGGCATTTTGG
TCCAAGTTGTGCTTATCCCATAGCCAGGAAACTCTGCATTCTAGTACTT
GGGAGACCTGTAATCATATAATAAATGTACATTAATTACCTTGAGCCAG
TAATTGGTCCGATCTTTGACTCTTTTGCCATTAAACTTACCTGGGCATT
CTTGTTTCAATTCCACCTGCAATCAAGTCCTACAAGCTAAAATTAGATG
AACTCAACTTTGACAACCATGAGACCACTGTTATCAAAACTTTCTTTTC
TGGAATGTAATCAATGTTTCTTCTAGGTTCTAAAAATTGTGATCAGACC
ATAATGTTACATTATTATCAACAATAGTGATTGATAGAGTGTTATCAGT
CATAACTAAATAAAGCTTGCAACAAAATTCTCTGACAAAAAAAAAAAAA
AAA.

As another example, human IL-1β is a polypeptide having the protein sequence:

(SEQ ID NO: 9)
MAEVPELASEMMAYYSGNEDDLFFEADGPKQMKCSFQDLDLCPLDGGIQ
LRISDHHYSKGFRQAASVVVAMDKLRKMLVPCPQTFQENDLSTFFPFIF
EEEPIFFDTWDNEAYVHDAPVRSLNCTLRDSQQKSLVMSGPYELKALHL
QGQDMEQQVVFSMSFVQGEESNDKIPVALGLKEKNLYLSCVLKDDKPTL
QLESVDPKNYPKKKMEKRFVFNKIEINNKLEFESAQFPNWYISTSQAEN
MPVFLGGTKGGQDITDFTMQFVSS,

which is encoded by the corresponding mRNA of the following cDNA sequence (the open reading frame is underlined):

(SEQ ID NO: 10)
ACCAAACCTCTTCGAGGCACAAGGCACAACAGGCTGCTCTGGGATTCTC
TTCAGCCAATCTTCATTGCTCAAGTGTCTGAAGCAGCCATGGCAGAAGT
ACCTGAGCTCGCCAGTGAAATGATGGCTTATTACAGTGGCAATGAGGAT
GACTTGTTCTTTGAAGCTGATGGCCCTAAACAGATGAAGTGCTCCTTCC
AGGACCTGGACCTCTGCCCTCTGGATGGCGGCATCCAGCTACGAATCTC
CGACCACCACTACAGCAAGGGCTTCAGGCAGGCCGCGTCAGTTGTTGTG
GCCATGGACAAGCTGAGGAAGATGCTGGTTCCCTGCCCACAGACCTTCC
AGGAGAATGACCTGAGCACCTTCTTTCCCTTCATCTTTGAAGAAGAACC
TATCTTCTTCGACACATGGGATAACGAGGCTTATGTGCACGATGCACCT
GTACGATCACTGAACTGCACGCTCCGGGACTCACAGCAAAAAAGCTTGG
TGATGTCTGGTCCATATGAACTGAAAGCTCTCCACCTCCAGGGACAGGA
TATGGAGCAACAAGTGGTGTTCTCCATGTCCTTTGTACAAGGAGAAGAA
AGTAATGACAAAATACCTGTGGCCTTGGGCCTCAAGGAAAAGAATCTGT
ACCTGTCCTGCGTGTTGAAAGATGATAAGCCCACTCTACAGCTGGAGAG
TGTAGATCCCAAAAATTACCCAAAGAAGAAGATGGAAAAGCGATTTGTC
TTCAACAAGATAGAAATCAATAACAAGCTGGAATTTGAGTCTGCCCAGT
TCCCCAACTGGTACATCAGCACCTCTCAAGCAGAAAACATGCCCGTCTT
CCTGGGAGGGACCAAAGGCGGCCAGGATATAACTGACTTCACCATGCAA
TTTGTGTCTTCCTAAAGAGAGCTGTACCCAGAGAGTCCTGTGCTGAATG
TGGACTCAATCCCTAGGGCTGGCAGAAAGGGAACAGAAAGGTTTTTGAG
TACGGCTATAGCCTGGACTTTCCTGTTGTCTACACCAATGCCCAACTGC
CTGCCTTAGGGTAGTGCTAAGAGGATCTCCTGTCCATCAGCCAGGACAG
TCAGCTCTCTCCTTTCAGGGCCAATCCCCAGCCCTTTTGTTGAGCCAGG
CCTCTCTCACCTCTCCTACTCACTTAAAGCCCGCCTGACAGAAACCACG
GCCACATTTGGTTCTAAGAAACCCTCTGTCATTCGCTCCCACATTCTGA
TGAGCAACCGCTTCCCTATTTATTTATTTATTTGTTTGTTTGTTTTATT
CATTGGTCTAATTTATTCAAAGGGGGCAAGAAGTAGCAGTGTCTGTAAA
AGAGCCTAGTTTTTAATAGCTATGGAATCAATTCAATTTGGACTGGTGT
GCTCTCTTTAAATCAAGTCCTTTAATTAAGACTGAAAATATATAAGCTC
AGATTATTTAAATGGGAATATTTATAAATGAGCAAATATCATACTGTTC
AATGGTTCTGAAATAAACTTCACTGAAG.

As another example, human IL-19 (isoform 1) is a polypeptide having the protein sequence:

(SEQ ID NO: 11)
MCTEGAFPHRSACSLPLTHVHTHIHVCVPVLWGSVPRGMKLQCVSLWLL
GTILILCSVDNHGLRRCLISTDMHHIEESFQEIKRAIQAKDTFPNVTIL
STLETLQIIKPLDVCCVTKNLLAFYVDRVFKDHQEPNPKILRKISSIAN
SFLYMQKTLRQCQEQRQCHCRQEATNATRVIHDNYDQLEVHAAAIKSLG
ELDVFLAWINKNHEVMFSA,

which is encoded by the corresponding mRNA of the following cDNA sequence (the open reading frame is underlined):

(SEQ ID NO: 12)
TGCACACACTGACAGGAGTCCAAGAATGTGCACTGAGGGAGCGTTTCCG
CACAGATCTGCGTGTTCCTTACCACTCACACATGTGCACACACATATCC
ATGTGTGTGTGCCAGTGCTTTGGGGCTCTGTTTCCACGGGGCATGAAGT
TACAGTGTGTTTCCCTTTGGCTCCTGGGTACAATACTGATATTGTGCTC
AGTAGACAACCACGGTCTCAGGAGATGTCTGATTTCCACAGACATGCAC
CATATAGAAGAGAGTTTCCAAGAAATCAAAAGAGCCATCCAAGCTAAGG
ACACCTTCCCAAATGTCACTATCCTGTCCACATTGGAGACTCTGCAGAT
CATTAAGCCCTTAGATGTGTGCTGCGTGACCAAGAACCTCCTGGCGTTC
TACGTGGACAGGGTGTTCAAGGATCATCAGGAGCCAAACCCCAAAATCT
TGAGAAAAATCAGCAGCATTGCCAACTCTTTCCTCTACATGCAGAAAAC
TCTGCGGCAATGTCAGGAACAGAGGCAGTGTCACTGCAGGCAGGAAGCC
ACCAATGCCACCAGAGTCATCCATGACAACTATGATCAGCTGGAGGTCC
ACGCTGCTGCCATTAAATCCCTGGGAGAGCTCGACGTCTTTCTAGCCTG
GATTAATAAGAATCATGAAGTAATGTTCTCAGCTTGATGACAAGGAACC
TGTATAGTGATCCAGGGATGAACACCCCCTGTGCGGTTTACTGTGGGAG
ACAGCCCACCTTGAAGGGGAAGGAGATGGGGAAGGCCCCTTGCAGCTGA
AAGTCCCACTGGCTGGCCTCAGGCTGTCTTATTCCGCTTGAAAATAGCC
AAAAAGTCTACTGTGGTATTTGTAATAAACTCTATCTGCTGAAAGGGCC
TGCAGGCCATCCTGGGAGTAAAGGGCTGCCTTCCCATCTAATTTATTGT
AAAGTCATATAGTCCATGTCTGTGATGTGAGCCAAGTGATATCCTGTAG
TACACATTGTACTGAGTGGTTTTTCTGAATAAATTCCATATTTTACCTA
TGAAAAAAAAAAAAAAAAAA.

As another example, human IL-19 (isoform 2) is a polypeptide having the protein sequence:

(SEQ ID NO: 13)
MKLQCVSLWLLGTILILCSVDNHGLRRCLISTDMHHIEESFQEIKRAIQ
AKDTFPNVTILSTLETLQIIKPLDVCCVTKNLLAFYVDRVFKDHQEPNP
KILRKISSIANSFLYMQKTLRQCQEQRQCHCRQEATNATRVIHDNYDQL
EVHAAAIKSLGELDVFLAWINKNHEVMFSA,

which is encoded by the corresponding mRNA of the following cDNA sequence (the open reading frame is underlined):

(SEQ ID NO: 14)
GCTGGAGTGCAATGGTGAAATTATAGCAGACTGCAGTCTTCAACTCCTG
ACCTCAAGCAATTGTCCTGCCTCCTCAACTTCCTGACTACAGGTGTGCA
TGAGGACTACAGGCAGGCATGTGCCAACACATGCAGCTTTTTTTTTTTT
TTTTTTTCAGAGATGTGGTCTCGCTTTGTTGCCTACACTGGTCTCAAAC
TCTTGGCCTCAAGGGATCCTCCCACCTCGGCTTCCCAAAGTGCAGAGAT
TACAGTCTCATTTTCTCTCTCTCTGCATTAATCAAGAATGAGAGAACCC
TCCAGGGGACAAGATGAAGGGGAAATAGATGATGTGCAAAGAAATCCTT
GCTTTATGAGGGGAAAAAGTGTTCCTCATGAAGTTCAACAAAATGATGC
AGGTAAAGCAGTTAGCTAGCACCTGGCACATGGCAGACACTCATAGCTG
CCTAAGGCATTGGAGAACTGGATCGTGCTGCAGCCAGAGGCACCTGCAG
AGCCTCATGGGCTGGCTGCTGCAGGGTGTGGCTGATTGAGAGTGCTTTT
GTGAGTTGGCCTGCAGGGTACACTTGGTAACGTGCCACAGCTCTCAGGA
AAGTGACCTAAGTTGGATTTTTCTGCATGGACATAGAATTGCAAAAAAT
TCTCATTTGCATGGAGATGGGGAGTTTATTTTTCCTAGAAGCTGCATGT
CAAGACCCAGAAGAAAGAGGCATTTCATAATAATGATTAATCAGCTATA
TCTTAAAGAAGAAAGAAAACAATTAAGGAAATACAATACTAAGAAAACA
AGGGGAAAAAACAATCTCCCCAAGGTGGATCCACCCAGCAAACCTTGAC
AGCATTTCCTCTTATCCACCTGAATAAAAATGACCAGCCCTTTCCAAAT
GGCAGAGAGCACTGAGAGGAGACACAAGGAGCAGCCCGCAAGCACCAAG
TGAGAGGCATGAAGTTACAGTGTGTTTCCCTTTGGCTCCTGGGTACAAT
ACTGATATTGTGCTCAGTAGACAACCACGGTCTCAGGAGATGTCTGATT
TCCACAGACATGCACCATATAGAAGAGAGTTTCCAAGAAATCAAAAGAG
CCATCCAAGCTAAGGACACCTTCCCAAATGTCACTATCCTGTCCACATT
GGAGACTCTGCAGATCATTAAGCCCTTAGATGTGTGCTGCGTGACCAAG
AACCTCCTGGCGTTCTACGTGGACAGGGTGTTCAAGGATCATCAGGAGC
CAAACCCCAAAATCTTGAGAAAAATCAGCAGCATTGCCAACTCTTTCCT
CTACATGCAGAAAACTCTGCGGCAATGTCAGGAACAGAGGCAGTGTCAC
TGCAGGCAGGAAGCCACCAATGCCACCAGAGTCATCCATGACAACTATG
ATCAGCTGGAGGTCCACGCTGCTGCCATTAAATCCCTGGGAGAGCTCGA
CGTCTTTCTAGCCTGGATTAATAAGAATCATGAAGTAATGTTCTCAGCT
TGATGACAAGGAACCTGTATAGTGATCCAGGGATGAACACCCCCTGTGC
GGTTTACTGTGGGAGACAGCCCACCTTGAAGGGGAAGGAGATGGGGAAG
GCCCCTTGCAGCTGAAAGTCCCACTGGCTGGCCTCAGGCTGTCTTATTC
CGCTTGAAAATAGCCAAAAAGTCTACTGTGGTATTTGTAATAAACTCTA
TCTGCTGAAAGGGCCTGCAGGCCATCCTGGGAGTAAAGGGCTGCCTTCC
CATCTAATTTATTGTAAAGTCATATAGTCCATGTCTGTGATGTGAGCCA
AGTGATATCCTGTAGTACACATTGTACTGAGTGGTTTTTCTGAATAAAT
TCCATATTTTACCTATGAAAAAAAAAAAAAAAAAA.

A suitable CD40 agonist (e.g., CD40L; agonistic anti-CD40 antibody (e.g., FGK4.5, BioXcell); etc.) and/or proinflammatory cytokine (e.g., TNFα, IL-1α, IL-1β, IL-19, interferon gamma (IFNγ), and the like) can also be a functional fragment thereof (i.e., the protein need not be the full length polypeptide). A suitable CD40 agonist (e.g., CD40L, agonistic anti-CD40 antibody, etc.) (or functional fragment thereof) and/or proinflammatory cytokine (e.g., TNFα, IL-1a, IL-1β, IL-19, interferon gamma (IFNγ), and the like)(or functional fragment thereof) can also be provide as a nucleic acid (e.g., DNA and/or mRNA) that encodes the polypeptide (e.g., full length, functional fragment, etc.).

Any convenient Toll-like receptor agonist can be used. Examples of suitable Toll-like receptor agonists (TLRs) include, but are not limited to: a CpG oligodeoxynucletide (CpG ODN)(a TLR-9 agonist); a natural Toll-like receptor ligand; conserved microbial products including (but not limited to) bacterial LPS and derivatives thereof, components of the bacterial cell wall (e.g., lipoteichoic acid), bacterial flagellin, microbial DNA, microbial single-stranded RNA, and viral double-stranded RNA; polyinosinic:polycytidylic acid (usually abbreviated “poly I:C”)(a TLR-3 agonist), heat-shock proteins (e.g., HSP60, HSP70); uric acid; surfactant protein A; the non-histone chromatin-binding protein high mobility group box 1 (HMGB1); the Ca2+- and Zn2+-binding protein S100A9; components and breakdown products of the extracellular matrix; and mitochondrial DNA (mtDNA). More information about Toll-like receptor agonists and examples of various Toll-like receptor agonists can be found in Vacchelli et al., Oncoimmunology. 2013 Aug. 1; 2(8):e25238. Epub 2013 Jun. 10; and U. S. patent applications 20130165455, and 20130084307; all of which are hereby incorporated by reference in their entirety. A CpG oligodeoxynucleotide (CpG ODN) is a nucleotide comprising a CpG motif (e.g., that binds to TLR-9). Any convenient CpG ODN can be used.

Any convenient indoleamine 2,3-dioxygenase (IDO) inhibitor can be used. Examples of suitable IDO inhibitors include, but are not limited to 1-methyl-DL-tryptophan (1MT); methyl-thiohydantoin-tryptophan (MTH-Trp); CAY10581 ((t)3,4-dihydro-3-hydroxy-2,2-dimethyl-4-[(phenylmethyl)amino]-2H-naphtho[2,3-p]pyran-5,10-dione); annulin B; and anti-IDO antibody; norharmane (9H-pyrido[3,4-b]indole); and the like. Information about IDO inhibitors and more examples of IDO inhibitors can be found, for example, in U.S. patent applications 20130289083, 20130123246, and 20120058079; all of which are hereby incorporated by reference in their entirety.

Any convenient compound (e.g., an antibody) that neutralizes checkpoint molecules (e.g., CTLA-4) can be used (i.e., a checkpoint molecule neutralizing compound). An exemplary antibody is an anti-CTLA-4 antibody (e.g., Ipilimumab). Checkpoint molecules include, but are not necessary limited to: CTLA-4 (Cytotoxic T Lymphocyte Antigen-4), PD-1 (CD279, Programmed Death-1, PDCD1), LAG-3 (Lymphocyte Activation Gene-3), PD-L1 (CD274), GITR (TNFRSF18, CD357), OX40 (CD134, TNFRSF4), and TIM-3 (T cell Immunoglobulin and Mucin protein-3). Thus, an antibody against any of the above checkpoint molecules can be used as an APC stimulatory agent (e.g., dendritic cell stimulatory agent). In some cases, the APC stimulatory agent is not an agent (e.g., an antibody) that neutralizes checkpoint molecules.

Contacting an APC, e.g., DC, with an APC stimulatory composition, e.g., dendritic cell stimulatory composition, in vivo can include introducing into an individual (administering to an individual, e.g., systemic or locally) an APC stimulatory composition, e.g., dendritic cell stimulatory composition. Contacting an APC, e.g., DC, with an APC stimulatory composition, e.g., dendritic cell stimulatory composition, can also be performed in vitro.

When an APC, e.g., DC, is contacted with an APC stimulatory composition, e.g., dendritic cell stimulatory composition, the contact can be for a period of time sufficient to stimulate uptake of an antigen by the APC, e.g., DC (thus producing a loaded APC, e.g., DC). In some cases, when an APC, e.g., DC, is contacted with an APC stimulatory composition, e.g., dendritic cell stimulatory composition, the contact can be for a period of time sufficient to stimulate the future uptake of an antigen by the APC, e.g., DC (thus producing an activated APC, e.g., DC, or a pre-activated APC, e.g., DC). In some cases, an APC, e.g., DC, is contacted with an APC stimulatory composition, e.g., dendritic cell stimulatory composition, for a period of time in a range of from 2 hours to 48 hours (e.g., 6 hours to 36 hours, 12 hours to 36 hours, 18 hours to 30 hours, 20 hours to 30 hours, 22 hours to 28 hours, 22 hours to 26 hours, 23 hours to 25 hours, or 24 hours).

In some cases, an APC, e.g., DC, is contacted with an APC stimulatory composition, e.g., dendritic cell stimulatory composition, prior to contact with a subject target antigen (e.g., in the absence of a subject target antigen) and/or a subject antibody composition (e.g., in the absence of a subject antibody composition). For example, in some cases, an APC, e.g., DC, is contacted with an APC stimulatory composition, e.g., dendritic cell stimulatory composition, for a period time in a range of from 2 hours to 48 hours (e.g., 6 hours to 36 hours, 12 hours to 36 hours, 18 hours to 30 hours, 20 hours to 30 hours, 22 hours to 28 hours, 22 hours to 26 hours, 23 hours to 25 hours, or 24 hours) prior to being contacted with a subject target antigen and/or a subject antibody composition. In other words, in some cases, an APC, e.g., DC, is contacted with an APC stimulatory composition, e.g., dendritic cell stimulatory composition, for a period time in a range of from 2 hours to 48 hours (e.g., 6 hours to 36 hours, 12 hours to 36 hours, 18 hours to 30 hours, 20 hours to 30 hours, 22 hours to 28 hours, 22 hours to 26 hours, 23 hours to 25 hours, or 24 hours) in the absence of a subject target antigen and/or a subject antibody composition.

As one illustrative example, in some cases, an APC stimulatory composition, e.g., dendritic cell stimulatory composition, is introduced into (i.e., administered to) an individual prior to administering a subject antibody composition, and thus the APC stimulatory composition, e.g., dendritic cell stimulatory composition, is introduced into the individual in the absence of a subject antibody composition. In some cases, an APC stimulatory composition, e.g., dendritic cell stimulatory composition, is introduced into (i.e., administered to) an individual after administering a subject antibody composition. In some cases, an APC stimulatory composition, e.g., dendritic cell stimulatory composition, is introduced into (i.e., administered to) an individual together with a subject antibody composition (e.g., administered simultaneously with, administered as part of the same composition, and the like). In some cases, a, APC, e.g., DC, is contacted with an APC stimulatory composition, e.g., dendritic cell stimulatory composition, in the presence of (e.g., while also being contacted with) a target antigen. In some cases (e.g., when the APC, e.g., DC, is a BMDC), an APC stimulatory composition, e.g., dendritic cell stimulatory composition, is not used (for either in vivo or in vitro methods). In some cases, an agent (e.g, Ft-3) that causes production and/or release of endogenous APC, e.g., DC, from bone marrow is administered to an individual prior to, simultaneous with, or after administration of a subject antibody composition.

Target Antigen.

Provided herein are methods that involve the contacting of an APC, e.g., DC, with a target antigen and the subsequent uptake (e.g., phagocytosis) of the antigen by the APC, e.g., DC. In some cases (e.g., where an antibody composition is administered to an individual), the APC, e.g., DC, is contacted in vivo with the target antigen. For example, the target antigen (e.g., cancer cells, a tumor, proteins, carbohydrates, or lipids expressed by the cancer cells, etc.) is present in the individual and the APC, e.g., DC, is also present in the individual, and the method involves the administration of an antibody composition (as described in more detail below) in order to facilitate the uptake of the target antigen. In some such cases, the method also involves administering to the individual a treatment that activates APCs, e.g., DCs, of the individual (as discussed above).

In some embodiments, an APC, e.g., DC, is contacted with a target antigen in vitro. In some such cases, APCs, e.g., DCs, are isolated from the individual or APCs, e.g., DCs, are derived from cells of the individual (e.g., derived from isolated monocytes of the individual). In either case, the APC, e.g., DC, is considered to be autologous to the individual. The APC, e.g., DC, is contacted in vitro with target antigen and with a subject antibody composition.

A target antigen can be any antigen which will be taken up by the APC, e.g., DC. If the antigen is a protein, the APC, e.g., DC, will process it and subsequently present certain peptide components to T cells. In some cases, a target antigen can be a polypeptide, a protein complex, a mixture of polypeptides, and the like. In some cases, the target antigen is a cell (e.g., a cell from the individual). For example, in some cases, contacting the APC, e.g., DC, comprises contacting an autologous APC, e.g., DC, with a cell (e.g., a cancer cell from the individual, e.g., a cell or cells from a tumor). In some cases, a target antigen is present in a complex mixture (e.g., a cellular lysate, a collection of plasma membrane proteins, etc.). Thus, in some embodiments, a target antigen is present in a cellular lystate. In some such cases, contacting the APC, e.g., DC, can include contacting the APC, e.g., DC, with a lysate from cancer cells of the individual (i.e., a cancer cell cellular lysate, a lysate enriched for plasma membrane proteins, a lysate containing plasma membrane proteins, etc.). Cancer cells of the individual, which can be the source of the target antigen (e.g., the source of a cellular lysate) or can be the target antigen, can be any cancer cell of the individual (e.g., cells from primary and/or metastatic tumors; cancerous cells from the blood; lymph node cells; cells from pleural effusions (e.g., malignant pleural effusions), e.g., from a patient with lung cancer; cells from peritoneal effusions (e.g., malignant peritoneal effusions), e.g., from a patient with ovarian cancer; the involved skin of patients with mycosis fungoides; etc.).

Target antigens can be tumor specific or tumor associated antigens (e.g., whole tumor or cancer cells, a tumor cell lysate, tumor cell membrane preparations (e.g., a membrane fraction), tumor cell plasma membrane preparations (e.g., a plasma membrane fraction), isolated or partially isolated antigens from tumors, fusion proteins, liposomes, and the like), viral particles or other preparations comprising viral antigens, and any other antigen or fragment of an antigen, e.g., a peptide or polypeptide antigen. The antigen can also be a bacterial cell, bacterial lysate, membrane fraction from a cellular lysate, or any other source. The antigen can be expressed or produced recombinantly, or even chemically synthesized. The recombinant antigen can also be expressed on the surface of a host cell (e.g., bacteria, yeast, insect, vertebrate or mammalian cells)(e.g., expressed on the plasma membrane), can be present in a lysate, or can be purified from the lysate. Alternatively, the antigen can be encoded by nucleic acids which can be ribonucleoic acid (RNA) or deoxyribonucleic acid (DNA), that are purified or amplified from a tumor cell.

A target antigen can be present in a sample from a subject. For example, a tissue sample from a hyperproliferative or other condition in a subject can be used as a source of antigen. Such a sample can be obtained, for example, by biopsy or by surgical resection. Such an antigen can be used as a lysate or as an isolated preparation. Alternatively, a membrane preparation of cells from a subject (e.g., a cancer patient), or an established cell line also can be used as an antigen or source of antigen or nucleic acid encoding the antigen.

In some embodiments, the target antigen to which the antibody of a subject antibody composition binds, is not the antigen which the APC, e.g., DC, subsequently presents to a T cell.

Antibody Composition.

A subject antibody composition can include at least one allogeneic IgG antibody that specifically binds to the target antigen. In some cases, the target antigen is not a checkpoint molecule. The terms “allogeneic antibody” or “alloantibody” are used herein to refer to an antibody that is not from the individual in question (e.g., an individual with a tumor and seeking treatment), but is from the same species, or is from a different species, but has been engineered to reduce, mitigate, or avoid recognition as a xeno-antibody (e.g., non-self). For example, the “allogeneic antibody” can be a humanized or super humanized antibody.

If a cancer cell of a human individual is contacted with an antibody that was not generated by that same person (e.g., the antibody was generated by a second human individual, the antibody was generated by another species such as a mouse, the antibody is a humanized antibody that was generated by another species, etc.), then the antibody is considered to be allogeneic (relative to the first individual). Likewise, if an APC, e.g., DC, from a first human individual is contacted with an antigen in the presence of an allogeneic antibody composition (i.e., a composition comprising at least one allogeneic antibody), the allogeneic antibody can be a human antibody (e.g., a humanized antibody, an antibody generated by a human, etc.), but the allogeneic antibody and the APC, e.g., DC, can be from different individuals (e.g., the allogeneic antibody can be from a second human individual; the allogeneic antibody can be an antibody from another species, where the antibody is humanized; etc.). In some embodiments, the APC (e.g., DC) is endogenous to an individual seeking, or undergoing cancer treatment by one or more methods described herein. A humanized mouse monoclonal antibody that recognizes a human antigen (e.g., a cancer-specific antigen, an antigen that is enriched in and/or on cancer cells, etc.) is considered to be an “alloantibody” (an allogeneic antibody) as used herein. For example, if a humanized monoclonal antibody is administered to a human individual, or is contacted with a cancer cell, the humanized monoclonal antibody is an allogeneic antibody because it is human (humanized), but the humanized monoclonal antibody is not from the same individual to whom it is being administered (the humanized monoclonal antibody is not from the same individual from whom the cancer cell is derived). Likewise, a fully human antibody that is generated by a mouse (e.g., via genome engineering that humanizes genomic loci in the mouse that are responsible for generating antibodies) would also be considered to be an allogeneic antibody.

In some cases, the allogeneic antibody does not significantly bind non-cancer antigens (e.g., the allogeneic antibody binds one or more non-cancer antigens with at least 10; 100; 1,000; 10,000; 100,000; or 1,000,000-fold lower affinity (higher Kd) than the target cancer antigen). In some cases, the target cancer antigen to which the allogeneic antibody binds is enriched on the cancer cell. For example, the target cancer antigen can be present on the surface of the cancer cell at a level that is at least 2, 5, 10; 100; 1,000; 10,000; 100,000; or 1,000,000-fold higher than a corresponding non-cancer cell. In some cases, the corresponding non-cancer cell is a cell of the same tissue or origin that is not hyperproliferative or otherwise cancerous.

In some cases, the allogeneic antibody binds an antigen that has a significant or detectable presence on the surface of a cancer cell. For example, the allogeneic antibody can bind to a target antigen that is present at an amount of at least 10; 100; 1,000; 10,000; 100,000; 1,000,000; 2.5×10⁶; 5×10⁶; or 1×10⁷ copies or more on the surface of a cancer cell.

In some cases, the allogeneic antibody binds an antigen on a cancer cell at a higher affinity than a corresponding antigen on a non-cancer cell. For example, the allogeneic antibody may preferentially recognize an antigen containing a polymorphism that is found on a cancer cell as compared to recognition of a corresponding wild-type antigen on the non-cancer cell. In some cases, the allogeneic antibody binds a cancer cell with greater avidity than a non-cancer cell. For example, the cancer cell can express a higher density of an antigen, thus providing for a higher affinity binding of a multivalent antibody to the cancer cell.

In addition, as used herein, the terms “allogeneic antibody” or “alloantibody” refer to IgG antibodies unless otherwise explicitly noted. Thus, “allogeneic antibody” and “alloantibody” are also referred to herein as “allogeneic IgG antibody”, “allo-IgG-antibody”, or “allo-IgG-Ab.”

In some cases, serum is used as a source of allogeneic IgG antibodies, in which cases the serum can be from a second individual (an individual other than the individual being treated). Thus, in some cases, polyclonal IgG antibodies are from serum (e.g., serum from a second individual). In some cases, an antibody composition having polyclonal allogeneic IgG antibodies with a plurality of binding specificities includes polyclonal allogeneic IgG antibodies that are pooled from 2 or more individuals (3 or more individuals, 4 or more individuals, 5 or more individuals, 6 or more individuals, 7 or more individuals, 8 or more individuals, 9 or more individuals, 10 or more individuals, etc.). In some cases, pooled serum is used as a source of alloantibody, in which cases the serum (e.g., pooled serum) can come from any number of individuals, none of whom are the first individual (e.g., the serum can be pooled from 2 or more individuals, 3 or more individuals, 4 or more individuals, 5 or more individuals, 6 or more individuals, 7 or more individuals, 8 or more individuals, 9 or more individuals, 10 or more individuals, etc.). As such, to pool antibodies from two or more individuals, the antibodies from each individual, or from sub-pools of serum from two or more individuals, can isolated/purified prior to pooling. On the other hand, serum can be pooled prior to antibody isolation/purification. In some cases, serum (e.g., pooled serum) can be used. In some cases, the antibodies are isolated/purified from serum prior to use. In some cases, a subject allogeneic antibody composition comprises 2 or more (e.g., 3 or more, 4 or more, 5 or more, 6 or more, 7 or more, 8 or more, 9 or more, 10 or more, 15 or more, 20 or more, 30 or more, 40 or more, 50 or more, 100 or more, 200 or more, 500 or more, 1000 or more, etc) allogeneic IgG antibodies. In some cases, the target antigen of at least one of the allogeneic IgG antibodies of a subject allogeneic antibody composition is unknown.

In some cases, the allogeneic antibody is a monoclonal antibody of a defined sub-class (e.g., IgG₁, IgG₂, IgG₃, or IgG₄). In some cases, a mixture of allogeneic antibodies is utilized in the methods, compositions, or kits of the present invention. In such cases, the allogeneic antibodies can be from a defined subclass, or can be a mixture of different subclasses. For example, the allogeneic antibodies can be IgG₂ antibodies. Various combinations of different subclasses, in different relative proportions, can be easily obtained by those of skill in the art. In some cases, a specific subclass, or a specific mixture of different subclasses can be particularly effective at cancer treatment or tumor size reduction. Such a subclass can be readily identified by assaying various subclasses and mixtures thereof for cancer treatment efficacy, e.g., as demonstrated in Example 4.

In some cases, the target antigen of at least one of the allogeneic IgG antibodies of a subject allogeneic antibody composition is known. For example, in some cases, one or more known antibodies are included in a subject antibody composition. For example, in some cases, a subject allogeneic antibody is an antibody that targets (specifically binds to) a target that is known to be enriched on/in particular cells and/or in patients with a particular condition. For example, in some cases an individual has a cancer, and the cancer in question is known to exhibit elevated levels of a particular antigen (e.g., a tumor-specific antigen, a cancer-specific antigen, a tumor-enriched antigen, a cancer enriched antigen, etc.). As an illustrative example, suitable such antibodies can include: an allogeneic anti-gp75 antibody, an allogeneic anti-MHC class I antibody, an allogeneic anti-CD20 antibody, an allogeneic anti-Her2 antibody (e.g., trastuzumab, Herceptin), and the like. Thus, in some cases, a subject allogeneic antibody can be an antibody that specifically binds a particular antigen, which can be any antigen expressed by a cancer cell (i.e., does not have to be, but can be, an antigen that is enriched in a cancer cell relative to other cells, and antigen that is unique to a cancer cell, etc.) (e.g., an allogeneic IgG antibody that specifically binds to an antigen of a cancer cell of the individual; a monoclonal antibody, such as a humanized monoclonal antibody, that specifically binds to an antigen of a cancer cell; a monoclonal antibody, such as a humanized monoclonal antibody, that specifically binds to a tumor-enriched antigen, a cancer-enriched antigen, a tumor-specific antigen, a cancer-specific antigen, etc.). In some cases, a subject antibody composition includes an allogeneic IgG antibody that specifically binds to an antigen of a cancer cell. In some such cases, the allogeneic IgG antibody is a monoclonal antibody (e.g., a humanized monoclonal antibody). In some cases, a subject allogeneic antibody composition includes one or more antibodies that target (specifically bind) any of the proteins listed in Table 2, or their orthologs (e.g., human orthologs) (see Example 2 below). For example, a subject allogeneic antibody composition can include one or more antibodies that target (specifically bind) one or more of the following proteins (or their orthologs, e.g., human orthologs) (accession identifier is in parentheses): ATP51 (Q06185), OAT (P29758), AIFM1 (Q9Z0X1), AOFA (Q64133), MTDC (P18155), CMC1 (Q8BH59), PREP (Q8K411), YMEL1 (088967), LPPRC (Q6PB66), LONM (Q8CGK3), ACON (Q99KI0), OD01 (Q60597), IDHP (P54071), ALDH2 (P47738), ATPB (P56480), AATM (P05202), TMM93 (Q9CQWO), ERG13 (Q9CQE7), RTN4 (Q99P72), CLO41 (Q8BQR4), ERLN2 (Q8BFZ9), TERA (Q01853), DAD1 (P61804), CALX (P35564), CALU (035887), VAPA (Q9WV55), MOGS (Q80UM7), GANAB (Q8BHN3), ERO1A (Q8R180), UGGG1 (Q6P5E4), P4HA1 (Q60715), HYEP (Q9D379), CALR (P14211), AT2A2 (055143), PDIA4 (P08003), PDIA1 (P09103), PDIA3 (P27773), PDIA6 (Q922R8), CLH (Q68FD5), PPIB (P24369), TCPG (P80318), MOT4 (P57787), NICA (P57716), BASI (P18572), VAPA (Q9WV55), ENV2 (P11370), VAT1 (Q62465), 4F2 (P10852), ENOA (P17182), ILK (055222), GPNMB (Q99P91), ENV1 (P10404), ERO1A (Q8R180), CLH (Q68FD5), DSG1A (Q61495), AT1A1 (Q8VDN2), HYOU1 (Q9JKR6), TRAP1 (Q9CQN1), GRP75 (P38647), ENPL (P08113), CH60 (P63038), and CH10 (Q64433).

For the sake of clarity, as discussed above with respect to the definition of the terms “specific binding,” “specifically binds,” and the like, a subject allogeneic IgG antibody that specifically binds to an antigen (a target antigen) of a cancer cell preferentially binds to that particular antigen relative to other available antigens. However, the target antigen need not be specific to the cancer cell or even enriched in cancer cells relative to other cells (e.g., the target antigen can be expressed by other cells). Thus, in the phrase “an allogeneic antibody that specifically binds to an antigen of a cancer cell,” the term “specifically” refers to the specificity of the antibody and not to the uniqueness of the antigen in that particular cell type. To avoid confusion, in some cases, the phrase “antibody that binds to an antigen of a cancer cell” is used herein, by which is meant an antibody that binds to an antigen of a cancer cell, but the antigen need not be specific to the cancer cell or even enriched in cancer cells relative to other cells.

In some cases, a subject composition includes 2 or more (e.g., 3 or more, 4 or more, 5 or more, 6 or more, 7 or more, 8 or more, 9 or more, 10 or more, 15 or more, 20 or more, 30 or more, 40 or more, 50 or more, 100 or more, 200 or more, 500 or more, 1000 or more, etc) allogeneic IgG antibodies, where at least two of the antibodies specifically bind to different antigens, and/or where at least two of the antibodies specifically bind to a different epitope of the same antigen. In some such cases, at least one of the two more allogeneic IgG antibodies are monoclonal antibodies (e.g., humanized monoclonal antibodies). In some such cases, at least two of the two more (at least 3 of the 3 or more, at least 4 of the 4 or more, at least 5 of the 5 or more, etc.) allogeneic IgG antibodies are monoclonal antibodies (e.g., humanized monoclonal antibodies).

In some cases, a subject antibody composition has one or more antibodies with unknown binding specificities (i.e., unknown target antigens) and one or more antibodies with known binding specificities (i.e., known target antigens). For example, in some cases, a subject antibody composition can be “spiked” with one or more allogeneic antibodies (e.g., 2 or more, 3 or more, 4 or more, 5 or more, 6 or more, 10 or more, etc.) that are each known to bind to an antigen that is known to be enriched in one or more cancers.

In some cases, a subject allogeneic antibody composition includes one or more antibodies selected from: an anti-gp75 antibody, and anti-MHC class I antibody, an anti-HLA antibody, an anti-CD20 antibody, and an anti-Her2 antibody (e.g., trastuzumab, Herceptin). In some cases, a subject allogeneic antibody composition includes one or more antibodies that target (specifically bind) one or more of the proteins listed in Table 2, or their orthologs (e.g., human orthologs) (see Example 2 below). For example, a subject allogeneic antibody composition can include one or more antibodies that target (specifically bind) one or more of the following proteins (or their orthologs, e.g., human orthologs) (accession identifier is in parentheses): ATP5I (Q06185), OAT (P29758), AIFM1 (Q9ZOX1), AOFA (Q64133), MTDC (P18155), CMC1 (Q8BH59), PREP (Q8K411), YMEL1 (088967), LPPRC (Q6PB66), LONM (Q8CGK3), ACON (Q99KI0), ODO1 (Q60597), IDHP (P54071), ALDH2 (P47738), ATPB (P56480), AATM (P05202), TMM93 (Q9CQWO), ERG13 (Q9CQE7), RTN4 (Q99P72), CLO41 (Q8BQR4), ERLN2 (Q8BFZ9), TERA (Q01853), DAD1 (P61804), CALX (P35564), CALU (035887), VAPA (Q9WV55), MOGS (Q80UM7), GANAB (Q8BHN3), ERO1A (Q8R180), UGGG1 (Q6P5E4), P4HA1 (Q60715), HYEP (09D379), CALR (P14211), AT2A2 (055143), PDIA4 (P08003), PDIA1 (P09103), PDIA3 (P27773), PDIA6 (Q922R8), CLH (Q68FD5), PPIB (P24369), TCPG (P80318), MOT4 (P57787), NICA (P57716), BASI (P18572), VAPA (Q9WV55), ENV2 (P11370), VAT1 (Q62465), 4F2 (P10852), ENOA (P17182), ILK (055222), GPNMB (Q99P91), ENV1 (P10404), ERO1A (Q8R180), CLH (Q68FD5), DSG1A (Q61495), AT1A1 (Q8VDN2), HYOU1 (Q9JKR6), TRAP1 (Q9CQN1), GRP75 (P38647), ENPL (P08113), CH60 (P63038), and CH10 (064433).

In some cases, a subject allogeneic antibody composition comprises IgGs from serum (e.g., serum from one individual or pooled serum as described above). In some cases, a subject allogeneic antibody composition comprises IgGs enriched from serum (e.g., serum from one individual or pooled serum as described above). In some such cases, the target antigens for some (e.g., greater than 0% but less than 50%), half, most (greater than 50% but less than 100%), or even all of the allogeneic antibodies (i.e., IgGs from the serum) are unknown. However, the chances are high that at least one antibody of the composition recognizes the subject target antigen of the method because such a composition contains a wide variety of antibodies specific for a wide variety of target antigens. In some such cases, the target antigens for at least one of the allogeneic IgG antibodies is unknown.

When a subject antibody composition includes 2 or more antibodies that have different binding specificities (i.e., bind to different epitopes of the same target, bind to different target antigens, etc.), the antibody composition is considered to have “polyclonal” antibodies (e.g., polyclonal allogeneic IgG antibodies with a plurality of binding specificities). For example, a composition having two or more monoclonal antibodies (e.g., where at least two of the antibodies bind to a different epitope of a common target, and/or where at least two of the antibodies bind to different target antigens) is considered to have polyclonal antibodies (e.g., polyclonal allogeneic IgG antibodies with a plurality of binding specificities). As such, a composition having “polyclonal allogeneic IgG antibodies with a plurality of binding specificities” encompasses a composition having two or more monoclonal antibodies. In some cases, a subject composition that comprises polyclonal allogeneic IgG antibodies with a plurality of binding specificities includes 2 or more (e.g., 3 or more, 4 or more, 5 or more, 6 or more, 7 or more, 8 or more, 9 or more, 10 or more, 15 or more, 20 or more, 30 or more, 40 or more, 50 or more, 100 or more, 200 or more, 500 or more, 1000 or more, etc) monoclonal antibodies (e.g., where at least two of the antibodies specifically bind to a different epitope of the same antigen, and/or where at least two of the antibodies specifically bind to different antigens).

In some cases, a subject antibody composition includes an allogeneic IgG antibody that is conjugated to an APC stimulatory agent, e.g., dendritic cell stimulatory agent (as described above, e.g., a TLR agonist, e.g., a CpG ODN; a proinflammatory cytokine; a CD40 agonist, and the like). In some cases, a subject antibody composition includes two or more antibodies that are conjugated to an APC stimulatory agent, e.g., dendritic cell stimulatory agent (as described above, e.g., a TLR agonist, e.g., a CpG ODN; a proinflammatory cytokine; a CD40 agonist, and the like). When an antibody is conjugated to another antibody (e.g., when a subject allogeneic IgG antibody is conjugated to an agonistic anti-CD40 antibody) the conjugated molecule can be in the form of a bi-specific antibody. In some such cases, the two or more antibodies are conjugated to the same an APC stimulatory agent, e.g., dendritic cell stimulatory agent. In some cases, the two or more antibodies are conjugated to different an APC stimulatory agents, e.g., dendritic cell stimulatory agents.

A subject allogeneic antibody composition can include serum or can include antibodies that have been enriched/purified from serum (e.g., via chromatography). In some cases, a subject antibody composition includes IgGs selected on the bases of their IgG subclass (e.g., IgG2), tumor-binding properties, and/or APC-activating (e.g., DC-activating) properties.

In some cases, the allogeneic antibody composition includes intravenous immunoglobulin (IVIG) and/or antibodies from (e.g., enriched from, purified from, e.g., affinity purified from) IVIG. IVIG is a blood product that contains IgG (immunoglobulin G) pooled from the plasma (e.g., in some cases without any other proteins) from many (e.g., sometimes over 1,000 to 60,000) normal and healthy blood donors. IVIG is commercially available. IVIG contains a high percentage of native human monomeric IVIG, and has low IgA content. When administered intravenously, IVIG ameliorates several disease conditions. Therefore, the United States Food and Drug Administration (FDA) has approved the use of IVIG for a number of diseases including (1) Kawasaki disease; (2) immune-mediated thrombocytopenia; (3) primary immunodeficiencies; (4) hematopoietic stem cell transplantation (for those older than 20 yrs); (5) chronic B-cell lymphocytic leukemia; and (6) pediatric HIV type 1 infection. In 2004, the FDA approved the Cedars-Sinai IVIG Protocol for kidney transplant recipients so that such recipients could accept a living donor kidney from any healthy donor, regardless of blood type (ABO incompatible) or tissue match.

In some cases where the allogeneic antibody composition includes IVIG or includes antibodies from IVIG, one or more of the antibodies in the composition are conjugated to an APC stimulatory agent, e.g., dendritic cell stimulatory agent (as described above, e.g., a TLR agonist, e.g., a CpG ODN; a proinflammatory cytokine; a CD40 agonist, and the like). In some cases where the allogeneic antibody composition includes IVIG or includes antibodies from IVIG, one or more of the antibodies in the composition is conjugated to a CD40 agonist (e.g., CD40L, an agonistic anti-CD40 antibody, etc.). In some cases where the allogeneic antibody composition includes IVIG or includes antibodies from IVIG, one or more of the antibodies in the composition is conjugated to a proinflammatory cytokine (e.g., TNFα, IL-1α, IL-1β, IL-19, interferon gamma (IFNγ), and the like) (as described below). In some cases where the allogeneic antibody composition includes IVIG or includes antibodies from IVIG, one or more of the antibodies in the composition is conjugated to a proinflammatory cytokine (e.g., TNFα, IL-1α, IL-1β, IL-19, interferon gamma (IFNγ), and the like) (as described below) and at least one antibody in the composition is conjugated to a CD40 agonist (e.g., CD40L, an agonistic anti-CD40 antibody, etc.). In some cases where the allogeneic antibody composition includes IVIG or includes antibodies from IVIG, at least one antibody in the composition is conjugated to a proinflammatory cytokine (e.g., TNFα, IL-1α, IL-1β, IL-19, interferon gamma (IFNγ), and the like) (as described below); at least one antibody in the composition is conjugated to a CD40 agonist (e.g., CD40L, an agonistic anti-CD40 antibody, etc.); and at least one antibody in the composition is conjugated to a CpG oligodeoxynucleotide (CpG ODN). When an antibody is conjugated to another antibody (e.g., when a subject allogeneic IgG antibody is conjugated to an agonistic anti-CD40 antibody) the conjugated molecule can be in the form of a bi-specific antibody. In some cases where the allogeneic antibody composition includes IVIG or includes antibodies from IVIG, one or more conjugated antibodies (conjugated with a dendritic cell stimulatory agent) are spiked (i.e. added into) the antibody composition such that the composition includes antibodies of the IVIG and one or more antibodies that are conjugated with an APC stimulatory agent, e.g., dendritic cell stimulatory agent.

For more information regarding IVIG, please refer to U.S. patent applications: 20100150942, 20040101909, 20130177574; 20130108619; 20130011388; all of which are hereby incorporated by reference in their entirety.

Contacting an APC, e.g., DC, to Produce a Loaded APC, e.g., Loaded DC.

In some embodiments, an APC, e.g., DC, is contacted with a target antigen and a subject antibody composition at a dose and for a period of time effective for the uptake of the target antigen by the APC, e.g., DC, thereby producing a loaded APC, e.g., DC. In some cases, the target antigen is contacted with the antibody composition (thus producing an immune complex) prior to contacting the APC, e.g., DC (e.g., in the absence of APC, e.g., DC). In some such cases, the target antigen and the antibody composition are contacted for a period of time in a range of from 5 minutes to 2 hours (e.g., from 5 minutes to 90 minutes, from 5 minutes to 60 minutes, from 10 minutes to 60 minutes, from 10 minutes to 50 minutes, from 10 minutes to 45 minutes, from 15 minutes to 45 minutes, from 20 minutes to 40 minutes, from 20 minutes to 40 minutes, from 25 minutes to 35 minutes, or 30 minutes).

The identity of the antigen(s) to which a subject antibody (or subject antibody composition) specifically binds is not necessarily a critical factor of the subject method (e.g., see the working examples below). In some cases, it is instead important that the cancer cell (e.g., a tumor, a tumor cell, etc.) be contacted with enough antibodies that an APC, e.g., DC, uptakes a target antigen (e.g., a cell of a tumor, a cancer cell, etc.). Thus, in some cases, an APC, e.g., DC, is contacted with an antibody composition (e.g., an effective amount of an antibody composition) where the antibody (or antibodies) is at a high enough concentration (i.e., an effective concentration) to stimulate the uptake of a target antigen by an APC, e.g., DC.

In some cases, the target antigen and the antibody composition are contacted where the allogeneic IgG antibodies are at an antibody concentration in a range of from 100 ng/ml to 100 μg/ml (e.g., 250 ng/ml to 75 μg/ml, 250 ng/ml to 50 μg/ml, 250 ng/ml to 25 μg/ml, 500 ng/ml to 25 μg/ml, 500 ng/ml to 15 μg/ml, 500 ng/ml to 10 μg/ml, 500 ng/ml to 5 μg/ml, 750 ng/ml to 3 μg/ml, 750 ng/ml to 2 μg/ml, or 1 μg/ml). In some cases (e.g., where the target antigen is a cell), the target antigen and the antibody composition are contacted where the allogeneic IgG antibodies are at an antibody concentration in a range of from 100 ng/ml to 100 μg/ml (e.g., 250 ng/ml to 75 μg/ml, 250 ng/ml to 50 μg/ml, 250 ng/ml to 25 μg/ml, 500 ng/ml to 25 μg/ml, 500 ng/ml to 15 μg/ml, 500 ng/ml to 10 μg/ml, 500 ng/ml to 5 μg/ml, 750 ng/ml to 3 μg/ml, 750 ng/ml to 2 μg/ml, or 1 μg/ml) per 1×10⁵ target cells (e.g., cancer cells from the individual).

In some cases, the antibody composition is contacted with 1×10² or more target cells (e.g., cancer cells from the individual) (e.g., 1×10³ or more cells, 1×10⁶ or more cells, 1×10⁵ or more cells, or 1×10⁶ or more cells). In some cases, the antibody composition is contacted with target cells (e.g., cancer cells from the individual) in a range of from 1×10² to 1×10¹⁰ cells (1×10² to 1×10⁸ cells, 1×10³ to 1×10⁷ cells, 1×10⁴ to 1×10⁶ cells, 5×10⁴ to 5×10⁵ cells, or 1×10⁵ cells).

In some cases, when the antibody composition and the target antigen (e.g., cells from the individual) are contacted prior to contacting the APC, e.g., DC, thus producing an immune complex, the immune complex can be contacted with the APC, e.g., DC. In some such cases, the immune complex can be contacted with the APC, e.g., DC, where the cells of the immune complex (cells from the individual that have been contacted with the antibody composition) are intact; while in other cases, the immune complex can be contacted with the APC, e.g., DC, where the cells of the immune complex (cells from the individual that have been contacted with the antibody composition) have been lysed, forming a lysate (i.e., an immune complex lysate).

In some cases, where the target antigen is a cell and a subject antibody composition will be contacted with the target antigen cells prior to contacting APC, e.g., DC (thus forming an immune complex), and where the cells remain intact, the APC, e.g., DC, can be contacted with 1×10² or more immune complex cells (e.g., cancer cells from the individual that have been contacted with a subject antibody composition) (e.g., 1×10³ or more cells, 1×10⁴ or more cells, 1×10⁵ or more cells, or 1×10⁶ or more cells). In some cases, where the target antigen is a cell and a subject antibody composition will be contacted with the target antigen cells prior to contacting APC, e.g., DC (thus forming an immune complex), and where the cells remain intact, the APC, e.g., DC, can be contacted with a number of immune complex cells (e.g., cancer cells from the individual that have been contacted with a subject antibody composition) in a range of from 1×10² to 1×10¹⁰ cells (1×10² to 1×10⁸ cells, 1×10³ to 1×10⁷ cells, 1×10⁴ to 1×10⁵ cells, 5×10⁴ to 5×10⁵ cells, or 1×10⁵ cells).

In some cases, where the target antigen is a cell and a subject antibody composition will be contacted with the target antigen cells prior to contacting APC, e.g., DC (thus forming an immune complex), and where the cells are lysed to produce a lysate immune complex, the APC, e.g., DC, can be contacted with lysate (e.g., a lysate having surface expressed antigens; an unfractionated lysate; a lysate that has been enriched for surface expressed antigens, i.e., plasma membrane expressed antigens; a membrane enriched fraction of a lysate; etc.) from 1×10² or more immune complex cells (e.g., cancer cells from the individual that have been contacted with a subject antibody composition) (e.g., 1×10³ or more cells, 1×10⁴ or more cells, 1×10⁵ or more cells, or 1×10⁶ or more cells). In some cases, where the target antigen is a cell and a subject antibody composition will be contacted with the target antigen cells prior to contacting APC, e.g., DC (thus forming an immune complex), and where the cells are lysed to produce alysate immune complex, the APC, e.g., DC, can be contacted with lysate from a number of immune complex cells (e.g., cancer cells from the individual that have been contacted with a subject antibody composition) in a range of from 1×10² to 1×10¹⁰ cells (1×10² to 1×10⁸ cells, 1×10³ to 1×10⁷ cells, 1×10⁴ to 1×10⁶ cells, 5×10⁴ to 5×10⁵ cells, or 1×10⁵ cells).

In some embodiments, an APC, e.g., DC, is contacted simultaneously with a target antigen and a subject antibody composition. In such cases, the same concentrations and cell numbers apply as were discussed above for cases where the target antigen and antibody composition are contacted prior to contacting the APC, e.g., DC.

In some embodiments, syngenic IgG (IgG antibodies isolated from the same individual from whom the target antigen is isolated/derived) can be used to load APC, e.g., DC. In general, this would not work because the individual is thought not to have circulating antibodies that bind to the target antigen. However, if antibodies from the individual can be “forced” to bind to the target antigen, then the product (still referred to herein as an immune complex) can be used to load APC, e.g., DC. For example, in some cases, a syngenic IgG antibody (e.g., a composition having polyclonal syngenic IgG antibodies) can be cross-linked to a target antigen (described above) to produce an immune complex. The produced immune complex can then be contacted with APC, e.g., DC (e.g., syngenic APC, e.g., DC, i.e., APC, e.g., DC, from the same individual who provided that target antigen and the antibody(ies)) to load the APC, e.g., DC.

In some cases, the methods include verifying that the APC, e.g., DC, have been loaded (i.e., verifying the presence of loaded APC, e.g., DC). Any convenient method for determining whether an APC, e.g., DC, is a loaded APC, e.g., DC, can be used. For example, in some cases, the morphology alone of the APC, e.g., DC, is indicative that the APC, e.g., DC, is loaded. In some cases, upregulation of MHCII (e.g., HLA-DR), CD40, and/or CD86 is indicative that an APC, e.g., DC, is loaded. For example, in some cases, upregulation of MHCII (e.g., HLA-DR) and/or CD86 is indicative that a DC is loaded. In some cases, upregulation of CD40 and/or CD86 is indicative that a DC is loaded. For example, an increase in the fraction (%) of DC that co-express CD40 and CD86 (sometimes referred to as “% CD40/CD86”); after contacting DC (e.g., with a tumor antigen, an antibody, a composition comprising polyclonal antibodies, a dendritic cell stimulatory composition, or any combination thereof); relative to the fraction prior to contact, or relative to the fraction in control DC (e.g., DC not contacted in the same way and/or with the same composition); can be considered to be indicative that DC are loaded. (see the Examples section below).

Contacting a T Cell with a Loaded APC, e.g., DC.

In some embodiments, a T cell is contacted with a loaded APC, e.g., DC. During contact, the loaded APC, e.g., DC, presents antigens to the T cell to produce a contacted T cell, and the contacted T cell generates an immune response specific to the presented antigens. The T cells can be CD4+ T cells, CD8+ T cells, or a combination of CD4+ and CD8+ T cells.

Contacting a T cell with a loaded APC, e.g., DC, can be in vitro or in vivo. Thus, the phrase “contacting a T cell” encompasses both in vitro and in vivo contact. If the contact is in vivo, loaded APCs, e.g., DCs, can be administered to the individual and the APCs, e.g., DCs, then contact endogenous T cells of the individual to induce an immune response. Thus, a step of “contacting a T cell of an individual with a loaded APC”, e.g., “contacting a T cell of an individual with a loaded DC,” when performed in vivo, can in some cases be written: “introducing into an individual a loaded DC.” For example, in some cases, a subject method includes: (a) contacting in vitro an APC, e.g., DC, from an individual with: (i) a target antigen; and (ii) an antibody composition comprising an allogeneic IgG antibody that specifically binds to the target antigen, at a dose and for a period of time effective for the uptake of the target antigen by the APC, e.g., DC, thereby producing a loaded APC, e.g., DC; and (b) introducing into the individual the loaded APC, e.g., DC. APCs, e.g., DCs, can be administered to the individual as described below for the “administering cells”.

In some cases, the subject methods can be performed in vivo. In some such cases, contact is in vivo, endogenous APC, e.g., DC, are loaded in vivo, and the loaded APC, e.g., DC, then contact T cells in vivo. Thus, the method can be carried out by in vivo administration (e.g., administration of an antibody composition, administration of an antibody composition in combination with a treatment that activates APCs, e.g., DCs, of the individual, e.g, administering an antibody composition in combination with an APC stimulatory composition, e.g., a dendritic cell stimulatory composition, comprising an APC stimulatory agent, e.g., dendritic cell stimulatory agent). For example, endogenous APC, e.g., DC (e.g., TADC), can be loaded in vivo by administering to an individual an antibody composition (as described above)(e.g., a composition that comprises polyclonal allogeneic IgG antibodies with a plurality of binding specificities) and providing a treatment that activates APCs, e.g., DCs (e.g, TADCs), of the individual (as defined above). For example, the treatment that activates dendritic cells of the individual can include administering to the individual a dendritic cell stimulatory composition comprising a dendritic cell stimulatory agent (e.g., (i) a Toll-like receptor (TLR) agonist; (ii) a CD40 agonist and a proinflammatory cytokine; (iii) a checkpoint molecule neutralizing compound; (iv) an indoleamine 2,3-dioxygenase (IDO) inhibitor; (v) an NFkB activator; (vi) a compound that opens calcium channels; (vii) a T cell-related co-stimulatory molecule; or (vIII) a combination thereof). Upon loading, the loaded DC contact endogenous T cells in vivo.

In some embodiments where the subject methods are performed in vivo, endogenous APC, e.g., DC (e.g., TADC), can be loaded in vivo by administering to an individual an antibody composition (as described above)(e.g., a composition that comprises polyclonal allogeneic IgG antibodies with a plurality of binding specificities) in combination with an APC stimulatory composition, e.g., dendritic cell stimulatory composition, comprising an APC stimulatory agent, e.g., dendritic cell stimulatory agent. In some cases, endogenous APC, e.g., DC (e.g., TADC), can be loaded in vivo by administering to an individual an antibody composition (as described above)(e.g., a composition that comprises polyclonal allogeneic IgG antibodies with a plurality of binding specificities) in combination with a CD40 agonist (e.g., CD40L). In some cases, endogenous APC, e.g., DC (e.g., TADC), can be loaded in vivo by administering to an individual an antibody composition (as described above)(e.g., a composition that comprises polyclonal allogeneic IgG antibodies with a plurality of binding specificities) in combination with a CD40 agonist (e.g., CD40L) and a proinflammatory cytokine (e.g., TNFα and/or IFNγ). In some cases, endogenous APC, e.g., DC (e.g., TADC), can be loaded in vivo by administering to an individual (i) an antibody composition that includes polyclonal allogeneic IgG antibodies with a plurality of binding specificities; in combination with (ii) a CD40 agonist (e.g., CD40L) and TNFα. In some cases, endogenous APC, e.g., DC (e.g., TADC), can be loaded in vivo by administering to an individual (i) an antibody composition that includes polyclonal allogeneic IgG antibodies with a plurality of binding specificities; in combination with (ii) a CD40 agonist (e.g., CD40L) and IFNγ. In some cases, endogenous APC, e.g., DC (e.g., TADC), can be loaded in vivo by administering to an individual an antibody composition (as described above)(e.g., a composition that comprises polyclonal allogeneic IgG antibodies with a plurality of binding specificities) in combination with a Toll-like receptor agonist (e.g., a CpG ODN, polyinosinic:polycytidylic acid (“poly I:C”, a TLR-3 agonist), etc.). In some cases, endogenous APC, e.g., DC (e.g., TADC), can be loaded in vivo by administering to an individual (i) an antibody composition that includes polyclonal allogeneic IgG antibodies with a plurality of binding specificities; in combination with (ii) a Toll-like receptor agonist. In some cases, endogenous APC, e.g., DC (e.g., TADC), can be loaded in vivo by administering to an individual (i) an antibody composition that includes polyclonal allogeneic IgG antibodies with a plurality of binding specificities; in combination with (ii) polyinosinic:polycytidylic acid. Upon loading, the loaded APC, e.g., DC (e.g., TADC), can then contact endogenous T cells in vivo.

If the contact is in vitro, then an autologous T cell (e.g., a population of autologous T cells) from the individual can be contacted with a loaded APC, e.g., DC, to produce a contacted T cell (e.g., a population of contacted T cells). A T cell can be contacted with a loaded APC, e.g., DC, for a period of time sufficient to activate the T cell such that the T cell with induce an immune response when administered to the individual. T cells (either prior to or after contact with a loaded APC, e.g., DC) can be expanded in vitro and/or modified (e.g., genetically modified) prior to being administered to the individual.

In some cases, a T cell is contacted in vitro with a loaded APC, e.g., DC, for a period of time in a range of from 5 minutes to 24 hours (e.g., 5 minutes to 18 hours, 5 minutes to 12 hours, 5 minutes to 8 hours, 5 minutes to 6 hours, 5 minutes to 4 hours, 5 minutes to 2 hours, 5 minutes to 60 minutes, 5 minutes to 45 minutes, 5 minutes to 30 minutes, 15 minutes to 18 hours, 15 minutes to 12 hours, 15 minutes to 8 hours, 15 minutes to 6 hours, 15 minutes to 4 hours, 15 minutes to 2 hours, 15 minutes to 60 minutes, 15 minutes to 45 minutes, 15 minutes to 30 minutes, 20 minutes to 18 hours, 20 minutes to 12 hours, 20 minutes to 8 hours, 20 minutes to 6 hours, 20 minutes to 4 hours, 20 minutes to 2 hours, 20 minutes to 60 minutes, 20 minutes to 45 minutes, 30 minutes to 18 hours, 30 minutes to 12 hours, 30 minutes to 8 hours, 30 minutes to 6 hours, 30 minutes to 4 hours, 30 minutes to 2 hours, 30 minutes to 60 minutes, 30 minutes to 45 minutes, 45 minutes to 18 hours, 45 minutes to 12 hours, 45 minutes to 8 hours, 45 minutes to 6 hours, 45 minutes to 4 hours, 45 minutes to 2 hours, 45 minutes to 60 minutes, 1 hour to 18 hours, 1 hour to 12 hours, 1 hour to 8 hours, 1 hour to 6 hours, 1 hour to 4 hours, 1 hour to 2 hours, or 1 hour to 90 minutes).

In some cases, a population of T cells (e.g., 1×10² or more cells (e.g., 1×10³ or more cells, 1×10⁴ or more cells, 1×10⁵ or more cells, or 1×10⁶ or more cells)) is contacted in vitro with a loaded APC, e.g., DC (e.g., a population of loaded APCs, e.g., DCs; a population having loaded APCs, e.g., DCs; etc.). In some cases, a population of T cells (e.g., in a range of from 1×10² to 1×10¹⁰ cells (1×10² to 1×10⁸ cells, 1×10³ to 1×10⁷ cells, 1×10⁴ to 1×10⁵ cells, 5×10⁴ to 5×10⁵ cells, or 1×10⁵ cells)) is contacted in vitro with a loaded APC, e.g., DC (e.g., a population of loaded APCs, e.g., DCs; a population having loaded APCs, e.g., DCs; etc.). In some cases, a T cell (e.g., a population of T cells) is contacted with a cell population (e.g., 1×10² or more cells (e.g., 1×10³ or more cells, 1×10⁴ or more cells, 1×10⁵ or more cells, or 1×10⁶ or more cells)) having loaded APCs, e.g., DCs (e.g., a cell population of loaded APCs, e.g., DCs). In some cases, a T cell (e.g., a population of T cells) is contacted with a cell population (e.g., in a range of from 1×10² to 1×10¹⁰ cells (1×10² to 1×10⁸ cells, 1×10³ to 1×10⁷ cells, 1×10⁴ to 1×10⁵ cells, 5×10⁴ to 5×10⁵ cells, or 1×10⁵ cells)) having loaded APCs, e.g., DCs (e.g., a cell population of loaded APCs, e.g., DCs).

The contacted T cell (e.g., cells of a contacted T cell population) can be administered to the individual as described below for the “administering cells”.

In some embodiments, an autologous APC, e.g., DC, from the individual is contacted with a subject APC stimulatory agent, e.g., dendritic cell stimulatory agent, to produce a stimulated APC, e.g., DC; an autologous target antigen (e.g., a cancer cell from the individual) is contacted with a subject antibody composition to produce an immune complex; and the stimulated APC, e.g., DC, is contacted with the immune complex, for a period of time and at a concentration effective to induce the uptake of the target antigen (e.g., the immune complex) by the stimulated APC, e.g., DC; thereby producing a loaded APC, e.g., DC; and the loaded APC, e.g., DC, is contacted with a T cell (as described in greater detail above) to produce a contacted T cell, and the contacted T cell generates an immune response specific to the presented antigens.

Administering Cells and/or Compositions.

In some cases, cells (e.g., loaded APCs, e.g., loaded DCs, loaded macrophages, loaded B-cells; APCs, e.g., DCs, macrophages, B-cells; and/or contacted T cells) are cultured for a period of time prior to transplantation (i.e., administration to the individual). Cells (e.g., loaded APCs, e.g., loaded DCs, loaded macrophages, loaded B-cells; APCs, e.g., DCs, macrophages, B-cells; and/or contacted T cells) can be provided to the individual (i.e., administered into the individual) alone or with a suitable substrate or matrix, e.g. to support their growth and/or organization in the tissue to which they are being transplanted (e.g., target organ, tumor tissue, blood stream, and the like). In some embodiments, the matrix is a scaffold (e.g., an organ scaffold). In some embodiments, 1×10³ or more cells will be administered, for example 5×10³ or more cells, 1×10⁴ or more cells, 5×10⁴ or more cells, 1×10⁵ or more cells, 5×10⁵ or more cells, 1×10⁶ or more cells, 5×10⁰ or more cells, 1×10⁷ or more cells, 5×10⁷ or more cells, 1×10⁸ or more cells, 5×10⁸ or more cells, 1×10⁹ or more cells, 5×10⁹ or more cells, or 1×10¹⁰ or more cells. In some embodiments, subject cells are administered into the individual on microcarriers (e.g., cells grown on biodegradable microcarriers).

Subject cells (e.g., loaded APCs, e.g., loaded DCs, loaded macrophages, loaded B-cells; APCs, e.g., DCs, macrophages, B-cells; and/or contacted T cells) and/or compositions (e.g., a subject antibody composition; a subject ACP stimulatory composition, e.g., dendritic cell stimulatory composition; a combination thereof) can be administered in any physiologically acceptable excipient (e.g., William's E medium), where the cells may find an appropriate site for survival and function (e.g., organ reconstitution). The cells and/or compositions (e.g., a subject antibody composition; a subject ACP stimulatory composition, e.g., dendritic cell stimulatory composition; a combination thereof) may be introduced by any convenient method (e.g., injection, catheter, or the like). The cells and/or compositions can be encapsulated into liposomes or other biodegradable constructs. In some cases, one or more of (a) a subject antibody composition (e.g., including an allogeneic IgG antibody, antibodies of the antibody compositions, etc.); and (b) a treatment that activates APCs, e.g., DCs, of the individual (e.g., an APC stimulatory agent, e.g., DC stimulatory agent); is administered in a liposome, a microparticle, or a nanoparticle.

The cells and/or compositions (e.g., a subject antibody composition; a subject ACP stimulatory composition, e.g., dendritic cell stimulatory composition) may be introduced to the subject (i.e., administered to the individual) via any of the following routes: parenteral, subcutaneous (s.c.), intravenous (i.v.), intracranial (i.c.), intraspinal, intraocular, intradermal (i.d.), intramuscular (i.m.), intralymphatic (i.l.), or into spinal fluid. The cells and/or compositions (e.g., a subject antibody composition, a subject dendritic cell stimulatory composition) may be introduced by injection (e.g., systemic injection, direct local injection, local injection into or near a tumor and/or a site of tumor resection, etc.), catheter, or the like. Examples of methods for local delivery (e.g., delivery to a tumor and/or cancer site) include, e.g., by bolus injection, e.g. by a syringe, e.g. into a joint, tumor, or organ, or near a joint, tumor, or organ; e.g., by continuous infusion, e.g. by cannulation, e.g. with convection (see e.g. US Application No. 20070254842, incorporated here by reference); or by implanting a device upon which cells have been reversibly affixed (see e.g. US Application Nos. 20080081064 and 20090196903, incorporated herein by reference).

In some cases, one or more of: (a) a subject antibody composition (e.g., including an allogeneic IgG antibody, antibodies of the antibody compositions, etc.); and (b) a treatment that activates APCs, e.g., DCs, of the individual (e.g., an APC stimulatory agent, e.g., DC stimulatory agent); is administered by local injection into or near a tumor and/or a site of tumor resection. In some cases, one or more of: (a) a subject antibody composition (e.g., including an allogeneic IgG antibody, antibodies of the antibody compositions, etc.); and (b) a treatment that activates APCs, e.g., DCs, of the individual (e.g., an APC stimulatory agent, e.g., DC stimulatory agent); is administered by local injection into or near a tumor and/or a site of tumor resection in a liposome, a microparticle, or a nanoparticle.

The number of administrations of treatment to a subject may vary. Introducing cells and/or compositions (e.g., a subject antibody composition; a subject APC stimulatory composition, e.g., dendritic cell stimulatory composition) into an individual may be a one-time event; but in certain situations, such treatment may elicit improvement for a limited period of time and require an on-going series of repeated treatments. In other situations, multiple administrations of cells and/or compositions (e.g., a subject antibody composition; a subject APC stimulatory composition, e.g., dendritic cell stimulatory composition) may be required before an effect is observed. As will be readily understood by one of ordinary skill in the art, the exact protocols depend upon the disease or condition, the stage of the disease and parameters of the individual being treated.

A “therapeutically effective dose” or “therapeutic dose” is an amount sufficient to effect desired clinical results (i.e., achieve therapeutic efficacy). A therapeutically effective dose can be administered in one or more administrations. For purposes of this disclosure, a therapeutically effective dose of cells (e.g., loaded APCs, e.g., DCs; contacted T cells; and the like) and/or compositions (e.g., a subject antibody composition; a subject APC stimulatory composition, e.g., dendritic cell stimulatory composition) is an amount that is sufficient, when administered to (e.g., transplanted into) the individual, to palliate, ameliorate, stabilize, reverse, prevent, slow or delay the progression of the disease state (e.g., tumor size, tumor growth, tumor presence, cancer presence, etc.) by, for example, inducing an immune response against antigenic cells (e.g., cancer cells).

In some embodiments, a therapeutically effective dose of cells (e.g., loaded APC, e.g., DC; contacted T cells; etc.) is 1×10³ or more cells (e.g., 5×10³ or more, 1×10⁴ cells, 5×10⁴ or more, 1×10⁵ or more, 5×10⁵ or more, 1×10⁶ or more, 2×10⁶ or more, 5×10⁶ or more, 1×10⁷ cells, 5×10⁷ or more, 1×10⁸ or more, 5×10⁸ or more, 1×10⁹ or more, 5×10⁹ or more, or 1×10¹⁰ or more).

In some embodiments, a therapeutically effective dose of cells is in a range of from 1×10³ cells to 1×10¹⁰ cells (e.g, from 5×10³ cells to 1×10¹⁰ cells, from 1×10⁴ cells to 1×10¹⁰ cells, from 5×10⁴ cells to 1×10¹⁰ cells, from 1×10⁵ cells to 1×10¹⁰ cells, from 5×10⁵ cells to 1×10¹⁰ cells, from 1×10⁸ cells to 1×10¹⁰ cells, from 5×10⁸ cells to 1×10¹⁰ cells, from 1×10⁷ cells to 1×10¹⁰ cells, from 5×10⁷ cells to 1×10¹⁰ cells, from 1×10⁸ cells to 1×10¹⁰ cells, from 5×10⁸ cells to 1×10¹⁰, from 5×10³ cells to 5×10⁹ cells, from 1×10⁴ cells to 5×10⁹ cells, from 5×10⁴ cells to 5×10⁹ cells, from 1×10⁵ cells to 5×10⁵ to cells, from 5×10⁵ cells to 5×10⁹ cells, from 1×10⁶ cells to 5×10⁹ cells, from 5×10⁸ cells to 5×10⁹ cells, from 1×10⁷ cells to 5×10⁹ cells, from 5×10⁷ cells to 5×10⁹ cells, from 1×10⁸ cells to 5×10⁹ cells, from 5×10⁸ cells to 5×10⁹, from 5×10³ cells to 1×10⁹ cells, from 1×10⁴ cells to 1×10⁹ cells, from 5×10⁴ cells to 1×10⁹ cells, from 1×10⁵ cells to 1×10⁹ cells, from 5×10⁵ cells to 1×10⁹ cells, from 1×10⁸ cells to 1×10⁹ cells, from 5×10⁸ cells to 1×10⁹ cells, from 1×10⁷ cells to 1×10⁹ cells, from 5×10⁷ cells to 1×10⁹ cells, from 1×10⁸ cells to 1×10⁹ cells, from 5×10⁸ cells to 1×10⁹, from 5×10³ cells to 5×10⁸ cells, from 1×10⁴ cells to 5×10⁸ cells, from 5×10⁴ cells to 5×10⁸ cells, from 1×10⁵ cells to 5×10⁸ cells, from 5×10⁵ cells to 5×10⁸ cells, from 1×10⁸ cells to 5×10⁸ cells, from 5×10⁸ cells to 5×10⁸ cells, from 1×10⁷ cells to 5×10⁸ cells, from 5×10⁷ cells to 5×10⁸ cells, or from 1×10⁸ cells to 5×10⁸ cells).

In some embodiments, the concentration of cells (e.g., loaded APCs, e.g., DCs; contacted T cells; and the like) to be administered is in a range of from 1×10⁵ cells/ml to 1×10⁹ cells/ml (e.g., from 1×10⁵ cells/ml to 1×10⁸ cells/ml, from 5×10⁵ cells/ml to 1×10⁸ cells/ml, from 5×10⁵ cells/ml to 5×10⁷ cells/ml, from 1×10⁶ cells/ml to 1×10⁸ cells/ml, from 1×10⁶ cells/ml to 5×10⁷ cells/ml, from 1×10⁸ cells/ml to 1×10⁷ cells/ml, from 1×10⁶ cells/ml to 6×10⁸ cells/ml, or from 2×10⁸ cells/ml to 8×10⁶ cells/ml).

In some embodiments, the concentration of cells (e.g., loaded APCs, e.g., DCs; contacted T cells; and the like) to be administered is 1×10⁵ cells/ml or more (e.g., 1×10⁵ cells/ml or more, 2×10⁵ cells/ml or more, 3×10⁵ cells/ml or more, 4×10⁵ cells/ml or more, 5×10⁵ cells/ml or more, 6×10⁵ cells/ml or more, 7×10⁵ cells/ml or more, 8×10⁵ cells/ml or more, 9×10⁵ cells/ml or more, 1×10⁶ cells/ml or more, 2×10⁶ cells/ml or more, 3×10⁸ cells/ml or more, 4×10⁶ cells/ml or more, 5×10⁶ cells/ml or more, 6×10⁶ cells/ml or more, 7×10⁶ cells/ml or more, or 8×10⁸ cells/ml or more).

The cells and/or compositions (e.g., a subject antibody composition; a subject APC stimulatory composition, e.g., dendritic cell stimulatory composition) of this disclosure can be supplied in the form of a pharmaceutical composition, comprising an isotonic excipient prepared under sufficiently sterile conditions for human administration. For general principles in medicinal formulation, the reader is referred to Cell Therapy: Stem Cell Transplantation, Gene Therapy, and Cellular Immunotherapy, by G. Morstyn & W. Sheridan eds, Cambridge University Press, 1996; and Hematopoietic Stem Cell Therapy, E. D. Ball, J. Lister & P. Law, Churchill Livingstone, 2000. Choice of the cellular excipient and any accompanying elements of the composition will be adapted in accordance with the route and device used for administration. The composition may also comprise or be accompanied with one or more other ingredients that facilitate the engraftment or functional mobilization of the cells. Suitable ingredients include matrix proteins that support or promote adhesion of the cells, or complementary cell types.

Cells of the subject methods (e.g., APC, e.g., DC; loaded APC, e.g., loaded DC; T cells; contacted T cells; etc.) may be genetically modified to enhance survival, control proliferation, and the like. Cells may be genetically altered by transfection or transduction with a suitable vector, homologous recombination, or other appropriate technique, so that they express a gene of interest. In some embodiments, a selectable marker is introduced, to provide for greater purity of the desired cell.

For further elaboration of general techniques useful in the practice of this disclosure, the practitioner can refer to standard textbooks and reviews in cell biology, tissue culture, and embryology. With respect to tissue culture and stem cells, the reader may wish to refer to Teratocarcinomas and embryonic stem cells: A practical approach (E. J. Robertson, ed., IRL Press Ltd. 1987); Guide to Techniques in Mouse Development (P. M. Wasserman et al. eds., Academic Press 1993); Embryonic Stem Cell Differentiation in Vitro (M. V. Wiles, Meth. Enzymol. 225:900, 1993); Properties and uses of Embryonic Stem Cells: Prospects for Application to Human Biology and Gene Therapy (P. D. Rathjen et al., Reprod. Fertil. Dev. 10:31, 1998).

### Kits

Also provided are kits for use in the subject methods. The subject kits include any combination of components and compositions for performing the subject methods. In some embodiments, a kit can include the following: a subject antibody composition (as described in detail above, e.g., a allogeneic IgG antibody, a composition of 2 or more allogentic IgG antibodies, etc.); an APC stimulatory composition, e.g., dendritic cell stimulatory composition (as described in detail above, including, e.g., an APC stimulatory agent such as a dendritic cell stimulatory agent, a macrophage stimulatory agent, a B-cell stimulatory agent; an APC stimulatory agent conjugated to an IgG antibody such as a dendritic cell stimulatory agent conjugated to an IgG antibody, a macrophage stimulatory agent conjugated to an IgG antibody, a B-cell stimulatory agent conjugated to an IgG antibody; and the like); components for the isolation, culture, survival, or administration of APC, e.g., DC, and/or T cells; reagents (e.g., buffers) for contacting an APC, e.g., DC; reagents (e.g., buffers) for contacting a T cell; reagents (e.g., buffers) for contacting a target antigen with a subject antibody composition to produce an immune complex; and any combination thereof.

In some embodiments, a subject kit includes assay reagents (e.g, an antibody for the detection of HLA-DR, an antibody for the detection of CD84, and the like) for use in a verifying step (e.g., verifying that an APC, e.g., DC, is a loaded APC, e.g., DC)

In some embodiments, the kit comprises (i) a compartment comprising an antibody composition comprising an allogeneic IgG antibody that binds to an antigen of a cancer cell; and (ii) at least one compartment comprising at least one APC stimulatory composition, wherein the APC stimulatory composition is a dendritic cell stimulatory composition, a macrophage stimulatory composition, or a B-cell stimulatory composition.

In addition to the above components, the subject kits may further include (in certain embodiments) instructions for practicing the subject methods. These instructions may be present in the subject kits in a variety of forms, one or more of which may be present in the kit. One form in which these instructions may be present is as printed information on a suitable medium or substrate, e.g., a piece or pieces of paper on which the information is printed, in the packaging of the kit, in a package insert, and the like. Yet another form of these instructions is a computer readable medium, e.g., diskette, compact disk (CD), flash drive, and the like, on which the information has been recorded. Yet another form of these instructions that may be present is a website address which may be used via the internet to access the information at a removed site.

### EXAMPLES

The following examples are put forth so as to provide those of ordinary skill in the art with a complete disclosure and description of how to make and use the present invention, and are not intended to limit the scope of what the inventors regard as their invention nor are they intended to represent that the experiments below are all or the only experiments performed. Efforts have been made to ensure accuracy with respect to numbers used (e.g. amounts, temperature, etc.) but some experimental errors and deviations should be accounted for. Unless indicated otherwise, parts are parts by weight, molecular weight is weight average molecular weight, temperature is in degrees Celsius, and pressure is at or near atmospheric. Standard abbreviations may be used, e.g., room temperature (RT); base pairs (bp); kilobases (kb); picoliters (pl); seconds (s or sec); minutes (m or min); hours (h or hr); days (d); weeks (wk or wks); nanoliters (nl); microliters (ul); milliliters (ml); liters (L); nanograms (ng); micrograms (ug); milligrams (mg); grams ((g), in the context of mass); kilograms (kg); equivalents of the force of gravity ((g), in the context of centrifugation); nanomolar (nM); micromolar (uM), millimolar (mM); molar (M); amino acids (aa); kilobases (kb); base pairs (bp); nucleotides (nt); intramuscular (i.m.); intraperitoneal (i.p.); subcutaneous (s.c.); and the like.

### Example 1

### Materials and Methods

### Mice

129S1/SvImJ mice, C57Bl/6 WT mice, CD-1 outbred mice, Balb/c, GFP transgenic mice [C57BL/6-Tg (UBC-GFP) 30Scha/J], and mice that develop inducible melanoma (B6.Cg-Braf^{tm1Mmcm}/Pten^{tm1Hwu}Tg (Tyr-cre/ERT2)13Bos/BosJ) were purchased from the Jackson Laboratory (Bar Harbor, Me.) and bred on-site. FcγR+(B6.129P2-Fcer1g^{tm1Rav}) mice were purchased from Taconic (Germantown, N.Y.). Mice were sorted randomly into groups before assigning treatment conditions. All mice were maintained in an American Association for the Accreditation of Laboratory Animal Care-accredited animal facility. All protocols were approved by the Stanford University Institutional Animal Care and Use Committee under protocol APLAC-17466.

### Cell Lines

Anti-CD4 (GK1.5) and anti-CD8 (2.43) hybridomas, the human cell lines MCF7 and PANC-1 and the mouse lines B16F10 (melanoma), 4T-1, LL2 (Lewis lung carcinoma) and RMA (lymphoma) were all purchased from the ATCC. LMP pancreas tumor cells were isolated from Kras^{G12D/+}; LSL-Trp53^{R172H/+}; Pdx-1-Cre mice as described¹³. Cells were cultured in DMEM (Gibco, Carlsbad, Calif.) supplemented with 10% heat-inactivated FCS, 2 mM L-glutamine, 100 U/mL penicillin and 100 μg/mL streptomycin (Gibco) under standard conditions.

### Preparation and In Vitro Studies of Mouse DC Subsets

BM mononuclear cells were negatively selected using a murine monocyte enrichment kit (Stem Cell Technologies, Vancouver Canada), and FSC^{lo}/SSC^{lo}/Gr1^{hi}/CD115^{hi}/MHCII^{neg} cells were sorted with a FACS Aria II (BD Biosciences). Monocytes were cultured for 4-5 days in the presence of 50 ng/ml GM-CSF (PeproTech) to generate DC. For TADC, tumors were digested in Hank's balanced salt solution (HBSS, Gibco) containing 5 mg/mL collagenase IV and 0.01 mg/mL DNase I (Sigma). Cells were applied on a Ficoll gradient and magnetically enriched using CD11b⁺ selection kits (StemCells) and Gr1^{neg}/CD11c⁺/MHCII⁺ cells were sorted by FACS. In some experiments TADC were activated with 50 ng/mL TNFα (PeproTech) and 500 ng/mL CD40L (PeproTech) recombinant mouse proteins.

### Preparation and In Vitro Studies of Human DC

Mononuclear cells from fresh BM aspirates and peripheral blood of matched healthy donors were purchased from AllCells (Alameda, Calif.). 10 cm long rib bones and 6 mL blood were obtained from 2 patients undergoing resection of malignant pleural mesothelioma. The study protocol was approved by Stanford's Institutional Review Board, and informed consent was obtained from all subjects. To generate BMDC, bones were then flushed with PBS and mononuclear cells were separated on Ficoll gradients. For both healthy and tumor patents, CD34⁺ cells were enriched using magnetic beads (Miltenyi) and cultured for 9-12 days in IMDM (Gibco) supplemented with 50 ng/mL GM-CSF and 20 ng/mL IL-4 (PeproTech). For blood derived-DC, CD14⁺ cells were enriched from blood mononuclear cells using magnetic beads (Miltenyi) and cultured for 7 days in IMDM (Gibco) supplemented with 50 ng/mL GM-CSF and 20 ng/mL of IL-4 (PeproTech). In other studies, blood-derived DC obtained from a patient with stage I lung carcinoma were treated overnight with 50 ng/mL human TNFα (PeproTech) and 1 μg/mL CD40L (PeproTech).

### Flow Cytometty

For cell surface staining, monoclonal antibodies conjugated to FITC, PE, PE-Cy7, PE-Cy5.5, APC-Cy7, eFluor 650, or Pacific Blue and specific for the following antigens were used: CD11b (M1/70), Gr-1 (RB6-8C5), F4/80 (BM8), B220 (RA3-6B2) from BioLegend (San Diego, Calif.) and CD115 (AFS98), CD80 (16-10A1), 1-Ab (AF6-120.1), CD40 (1C10), CD86 (GL1) and CD40L (MR1) from eBioscience (San Diego, Calif.). For protein phosphorylation-specific flow cytometry, cells were activated for 5, 15 or 30 min with or without IC and fixed for 15 min with 1.8% paraformaldehyde. Cells were washed twice with PBS containing 2% FCS and incubated with 95% methanol at 4° C. for 20 min. Conjugated antibodies against phospho-p38 MAPK (Thr180/Tyr182), phospho-Akt (Thr308) and phospho-c-Jun (Ser63) were purchased from Cell Signaling and phospho-ERK1/2 (p44) (pT202/pY204) from BD Biosciences (San Jose, Calif.). For tumor-binding IgM and IgG, PE-conjugated anti-mouse IgM (RMM-1), anti-mouse IgG (Poli4052) and anti-human IgG (HP6017) were purchased from BioLegend. Flow cytometry was performed on a LSRII (BD Biosciences) and datasets were analyzed using FlowJo software (Tree Star, Inc.).

### Cytokine Measurements

Cells were seeded at 1×10⁶ cells/mL and cultured for 12 h with or without tumor immune complexes, or LPS (Sigma). TNFα, IFNγ, and IL-12 (p40/p70) in the supematants were measured by ELISA, according to manufacturer's instructions (R&D Systems, Minneapolis, Minn.).

### IgG and IgM Purification and Measurement

Mouse antibodies were obtained from pooled 5 mL 20-24-week-old mouse serum by liquid chromatography on AKTA Explorer/100Air (GE Healthcare). Total mouse IgG and IgM were purified using protein-G and 2-mercaptopyridine columns, respectively (GE Healthcare). The levels of purified IgG and IgM were measured with specific ELISA kits (Bethyl, Montgomery, Tex.) according to manufacturer's instructions.

### Preparation of Antibody-Tumor Lysate Immune Complexes (Ig-IC) and Antibody-Bound Tumor Cells

Tumor cells were fixed in 2% paraformaldehyde, stained with CFSE and washed extensively. For surgical resections, tumors were initially isolated after enzymatic digestion and sorted as FSC^{hi}/CD45^{neg} cells prior to their fixation and staining. To obtain Ig-IC, tumor cells were incubated for 30 min on ice with 1 μg syngeneic or allogeneic IgG or IgM per 1×10⁵ tumor cells. Cells were then washed from excess antibodies and used as such, or further disrupted with non-denaturing lysis buffer to obtain Ig-IC.

### Membrane Protein Extraction

For native membrane proteins extraction, tumors were suspended in SEAT buffer (pH 7.4, 250 mM sucrose, 10 mM triethanolamine, 1 mM EDTA, 10 mM acetic acid, protease inhibitor cocktail I-Sigma) and were homogenized in a dounce homogenizer. Lysates were spun twice at 900g for 5 min at 4° C. and the supernatant was transferred to a fresh tube and spun at 100,000×g for 1 h at 4C. The membrane pellet was resuspended in H₂O and, in some experiments, denatured or deglycosylated before use. For denatured membrane protein extraction, the membrane pellet was resuspended in 500 μl Radio-Immuno-Precipitation Assay buffer (RIPA, Sigma) and lysed with a 25G needle syringe. Lysates were incubated at 4° C. for 1h and spun at 100,000×g, 30 min, 4C. Supernatant containing detergent solubilized membrane proteins was collected and boiled for 5 min at 95° C. Deglycosylation of membrane proteins was performed using a commercial kit (New England Biolabs, Ipswich, Mass.) according to the manufacturer's instructions.

### In Vivo Tumor Models

For tumor transfer studies, 1×10⁵ LMP or B16 tumor cells were injected subcutaneously (s.c.) above the right flank and tumor development was measured twice a week with calipers. In some experiments, tumor cells were labeled with 25 M CFSE according to manufacturer's instructions (Invitrogen). For prophylactic immunization, mice were injected twice s.c., 7 days apart, with 2×10⁶ DC or monocytes that were loaded with tumor lysates or IC. For tumor recurrence studies, 2×10⁵ tumor cells were injected s.c. above the right flank, and the size of growing tumors was measured using calipers. When tumors reached 45-55 mm² for LMP and 12-16 mm² for B16, mice were anesthetized and visible macroscopic tumor was surgically removed. Resected tumors were enzymatically digested with 0.1 mg/mL of DNase I (Sigma) and 5 mg/mL collagenase IV (Sigma) in PBS. Cells were then fixed in 2% paraformaldehyde for 20 min, washed extensively in PBS and added, with or without purified mouse antibodies, to DC subsets. After overnight incubation, cells were washed, and 2×10⁶ were injected s.c. to tumor-resected mice. In some experiments 200 ng TNFα (Peprotech) and 1 μg CD40L, CD28, OX-40 (R&D), 2 μg LPS, or 200 μg polyl:C (Invivogen) in combination with 200 μg mouse IgG, were injected directly into tumors for two cycles of two consecutive days separated by a week. For metastases experiments, 1×10⁵ 4T-1 cells were injected into the mammary fat pad of syngeneic Balb/c mice. After 16 days, once tumors metastasized into the draining lymph node, the primary tumor nodules were injected 3 times (2 days apart) with IgG derived from CD-1 mice along with TNFα and CD40L.

### In Vivo Cell Depletion

Depletion of CD4⁺ and CD8⁺ T cells was achieved by intraperitoneal (i.p.) injection of 500 μg/mouse GK1.5 (anti-CD4) and 2.43 (anti-CD8) monoclonal antibodies, respectively, 3 days before tumor inoculation and every 3 days thereafter. For B cell depletion, 300 μg/mouse anti-CD19 and 300 μg/mouse anti-B220 (BioXcell, West Lebanon, N.H.) were injected i.p. 5 and 2 days before tumor inoculation and every 3 days thereafter. For NK cell depletion, mice were injected i.p. with 50 μl anti-asialo GM1 polydonal antibodies (Wako Chemicals Richmond, Va.), or with 200 μg anti-NK1.1 PK136 (BioXCell) on days −2, 0, 4, and 8 relative to tumor challenge. Individual mice were bled on days 0, 7, 14 and 21 and the levels of NK1.1*/CD3ε^{neg} cells were determined by flow cytometry to confirm depletion.

### Adoptive Transfer

Mice were injected i.v. with 1 mg/mouse of syngeneic or allogeneic IgG or IgM one day prior to tumor challenge and once again with tumor injection. For T cell transfer, CD4⁺ and CD8⁺ T cells were negatively selected using a murine enrichment kit (Stem Cell Technologies) and 5×10⁶ cells were injected i.v. to recipient mice one day before tumor challenge. Prior to their transfer tumor-associated cell subsets were enriched as follows: TADC were isolated by enrichment of MHCII⁺ cells on magnetic beads (Miltenyi) and subsequent sorting of Gr1^{neg}/CD11c⁺/CD64^{dull} by FACS. Tumor macrophages (TAM) were enriched with CD11b⁺ magnetic beads (Miltenyi) followed by sorting of Gr1^{neg}/CD64^{hi} cells. B cells were enriched with CD19⁺ magnetic beads (Miltenyi). NK cells were enriched with NK1.1⁺ magnetic beads (Miltenyi) and mast cells were enriched with c-kit⁺ magnetic beads (Miltenyi). For each cell subset, 2×10⁶ cells were injected s.c. into naive mice 3 days before being challenged with 4×10⁴ B16 tumor cells.

### T Cell Proliferation

3×10⁴ DC were co-cultured with 3×10⁵ MACS-enriched CD4⁺ T cells (Miltenyi, Germany) from spleens of LMP- or B16-immunized mice. After 6 days, cells were pulsed with ³H-thymidine (1 μCi/well) and cultured for an additional 18h before being harvested in a Harvester 400 (Tomtec). Radioactivity was measured by a 1450 MicroBeta counter (LKB Wallac).

### Immunofluorescence

DC or monocytes were incubated on glass-bottom culture plates (In Vitro Scientific) with CFSE-labeled tumor cells and with or without antibodies overnight. Cells were gently washed with PBS (Gibco), fixed for 20 min with 2% paraformaldehyde and permeabilized with 0.5% saponin (Sigma). Samples were blocked with 10% non-immune goat serum and stained with Alexa-conjugated anti-mouse IgG and IgM (Invitrogen 1:100) and anti-mouse 1-Ab (BD Biosciences, 1:100).

### Immunohistochemistry

Specimens were fixed in 4% paraformaldehyde, equilibrated in a 20% sucrose solution and embedded in frozen tissue matrix (Tissue-Tek OCT, Torrance, Calif.). Slides were cut to 5 μm, blocked with 10% non-immune goat serum and stained with Alexa-conjugated Rabbit anti-CD4 (RM4-5, eBioscience, 1:100), anti-CD8p (YTS156.7.7 BioLegend, 1:100), goat anti-mouse IgG (Invitrogen 1:100) and anti-mouse IgM (II/41 eBioscience, 1:100). Sections were examined under a Zeiss Laser Scanning Confocal Microscope. Images were collected using a Zeiss 700 confocal laser scanning microscope and analyzed using ZEN software (Carl Zeiss Microscopy).

### Statistics

Sample size was chosen such that statistical significance could be achieved using appropriate statistical tests (e.g. ANOVA) with errors approximated from previously reported studies. A non-parametric Mann-Whitney U test was performed in Prism (GraphPad Software, Inc.) to analyze experimental data, unless otherwise stated. Phospho-specific flow cytometry data were transformed by taking the inverse hyperbolic sine (arcsinh), and ratios were taken over the corresponding baseline (unstimulated) value as previously described (Irish et al., PNAS, 2010). No blinded experiments were performed. No samples were excluded from analyses. P values indicate significance of the difference between experimental and control (CT) values. *p<0.05; **p<0.01. Error bars represent ⁺/− SEM.

Results

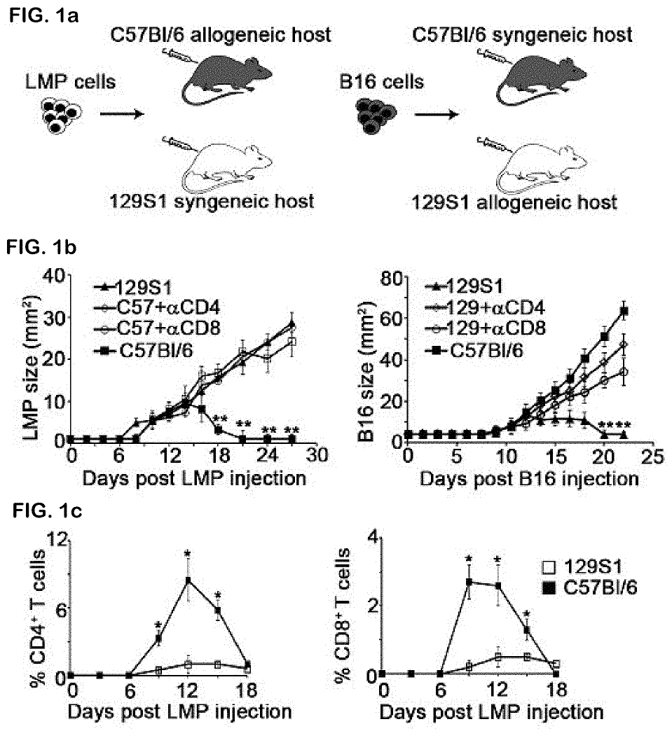

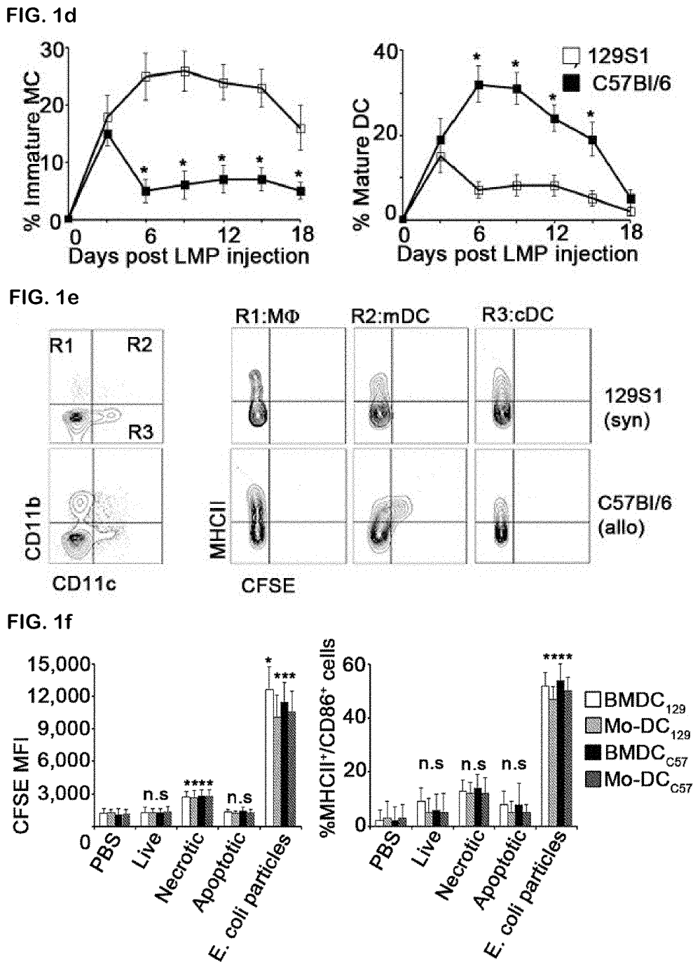

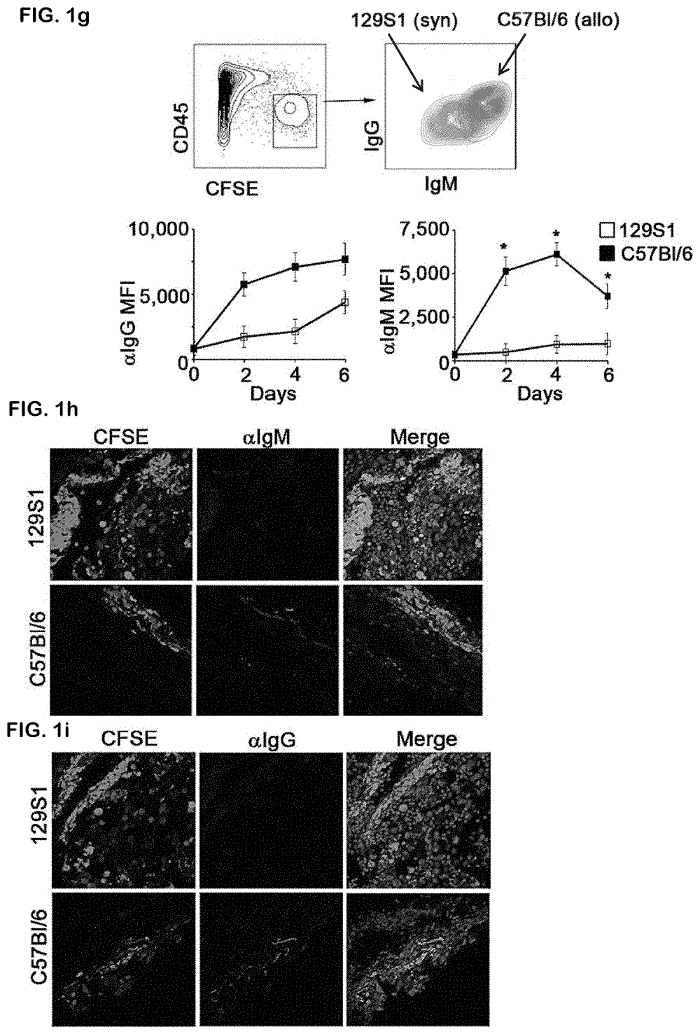

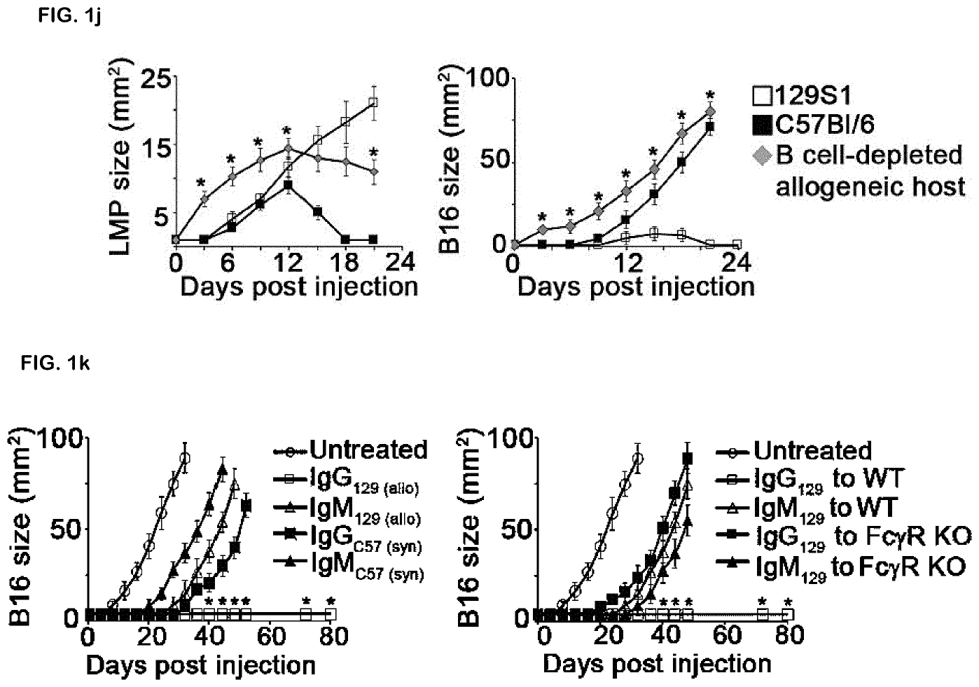

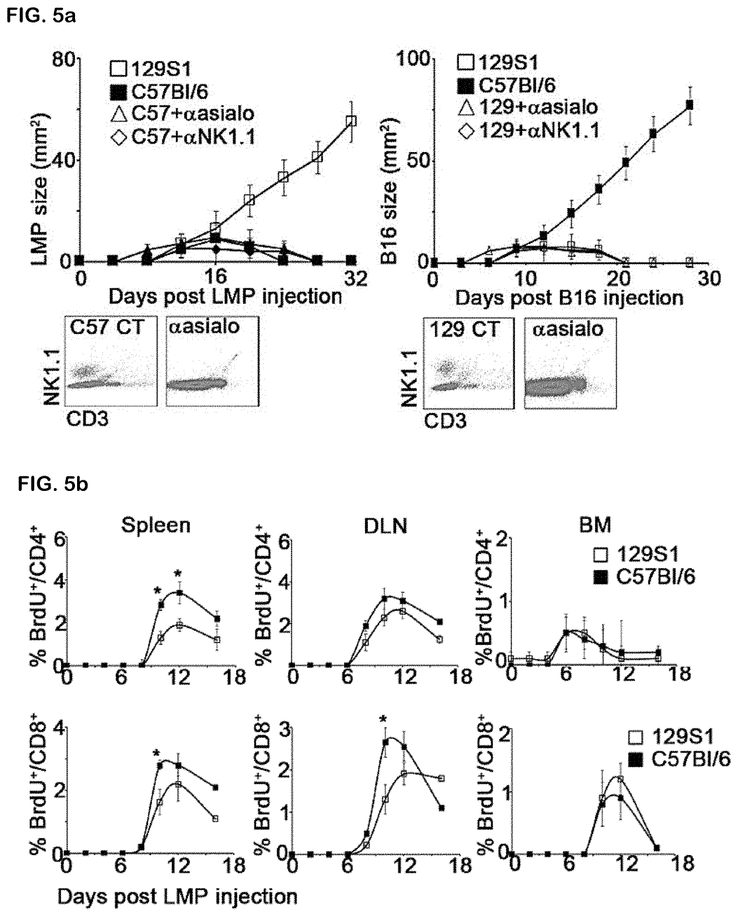

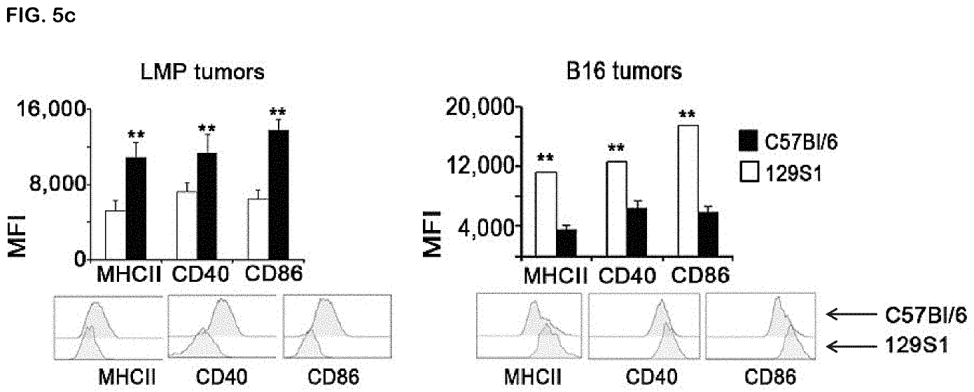

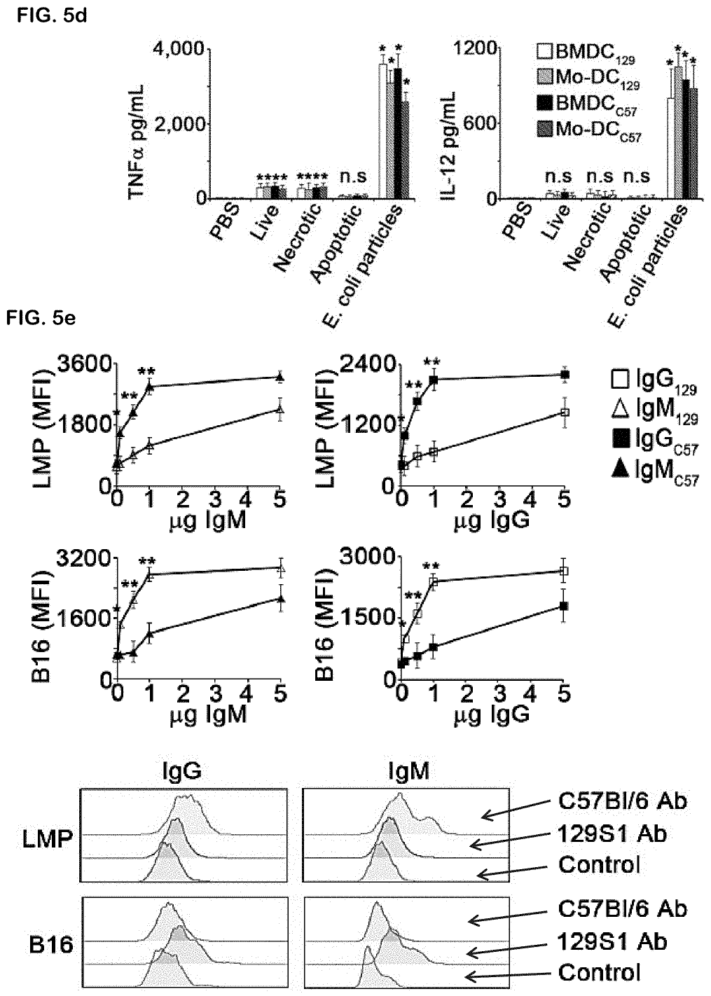

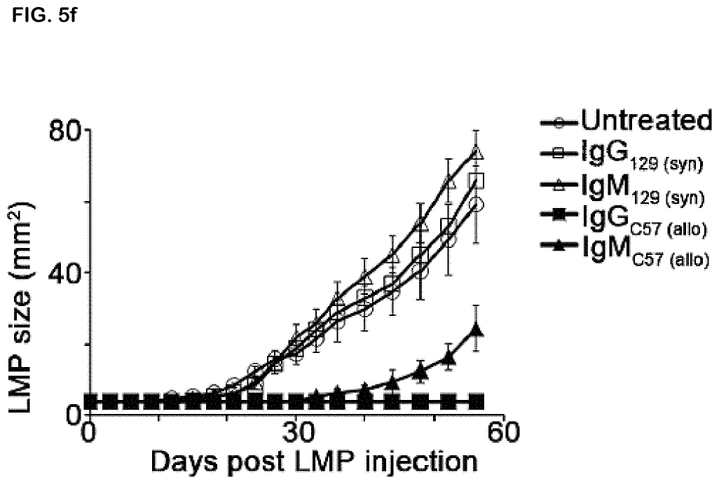

To study the cellular basis of allogeneic tumor rejection, the immune response to tumors in MHC matched, but otherwise genetically distinct, C57Bl/6 and 129S1 mice were compared (illustrated in FIG. 1a). B16 melanoma cells expanded continuously in syngeneic C57Bl/6 hosts yet spontaneously regressed in allogeneic 129S1 hosts (FIG. 1b). Conversely, LMP pancreatic tumor cells, isolated from Kras^{G12D/+}; LSL-Trp53^{R172H/+}; Pdx-1-Cre mice¹³, grew steadily in 129S1 mice but spontaneously regressed in C57Bl/6 animals (FIG. 1b). In both models, depletion of NK cells did not prevent tumor rejection (FIG. 5a). In contrast, host T cells played a requisite role in allogeneic tumor rejection, as depletion of CD4⁺ or CD8⁺ T cells prior to allogeneic tumor inoculation prevented tumor regression (FIG. 1b). T cell proliferation and infiltration of allogeneic tumors began at about 1 week and peaked at 10-12 days (FIG. 1c and FIG. 5b). In addition, allogeneic tumors contained more mature myeloid DC (mDC; Gr1^{neg}/CD11b⁺/CD11c⁺/MHCII⁺/CD64^{dim}) and fewer immature myeloid cells (iMC; Gr1^{hi}/CD11b^{hi}/MHCII^{neg/lo}) than syngeneic tumors (FIG. 1d). Moreover, DC in allogeneic tumors expressed higher levels of MHCII, CD86 and CD40 compared to DC in syngeneic tumors, reflecting a more activated phenotype (Figure Sc). After inoculating animals with allogeneic LMP cells labeled with CFSE, mDC also internalized tumor cell-derived molecules, suggesting that they might process and present tumor-associated antigens under these conditions (FIG. 1e). However, co-culture with allogeneic tumor cells induced little or no DC activation and uptake of tumor antigens, and no differential response relative to syngeneic DC (FIG. 1f, FIG. 5d), demonstrating that additional factors present in vivo are required to facilitate efficient tumor antigen internalization and DC activation.

Because antibodies can promote antigen uptake by DC via Fc receptor-mediated endocytosis of immune complexes (IC), the presence of tumor-binding antibodies was tested. IgM and IgG antibodies were bound to allogeneic, but not syngeneic, tumor cells within 24 hours following tumor inoculation (FIG. 1g-i), before the appearance of T cells (FIG. 1c). Moreover, allogeneic antibodies bound tumor cells significantly more effectively than syngeneic antibodies in culture (Figure Se). To assess the potential role of antibodies in tumor rejection, B cells were depleted from allogeneic hosts. Once the levels of circulating IgG and IgM dropped below 180 and 10 μg/mL, respectively, mice were challenged with allogeneic tumors. B cell depletion accelerated tumor development relative to untreated hosts and delayed or prevented tumor rejection (FIG. 1j). Moreover, adoptive transfer of allogeneic IgG, but not IgM, enabled rejection of syngeneic tumors (FIG. 1k and FIG. 5f). This effect was almost completely abrogated in mice deficient in Fc gamma receptors (FcγR) (FIG. 1k). These results suggest an essential role for allogeneic antibody-dependent signaling in the induction of tumor-eradicating immune responses.

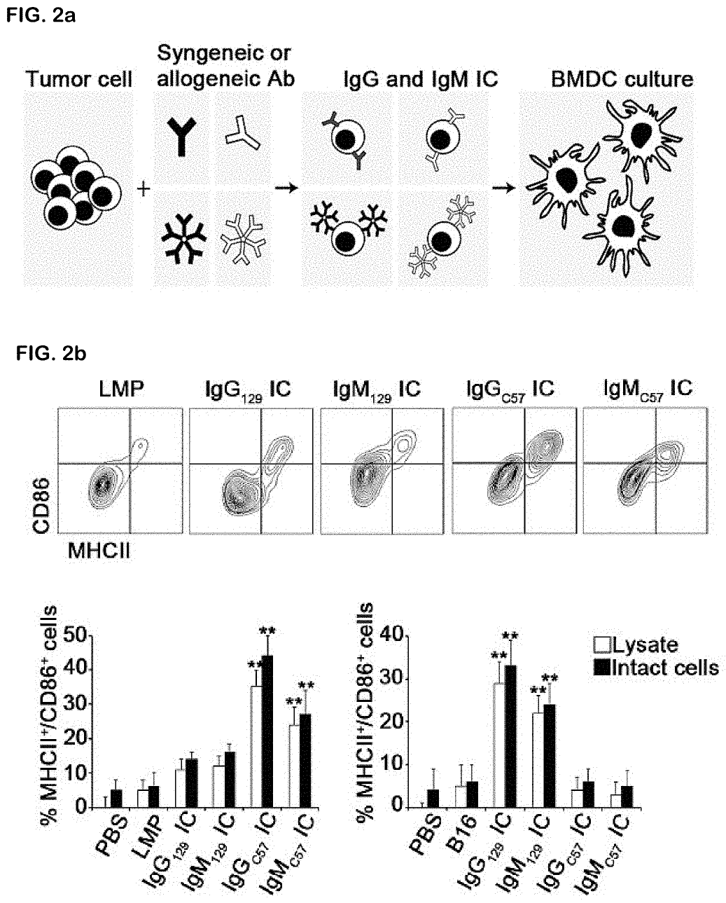

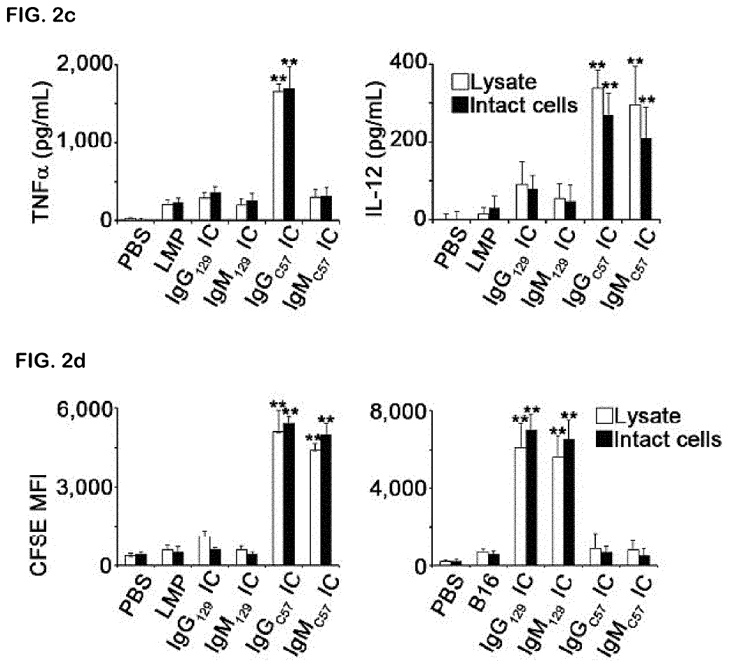

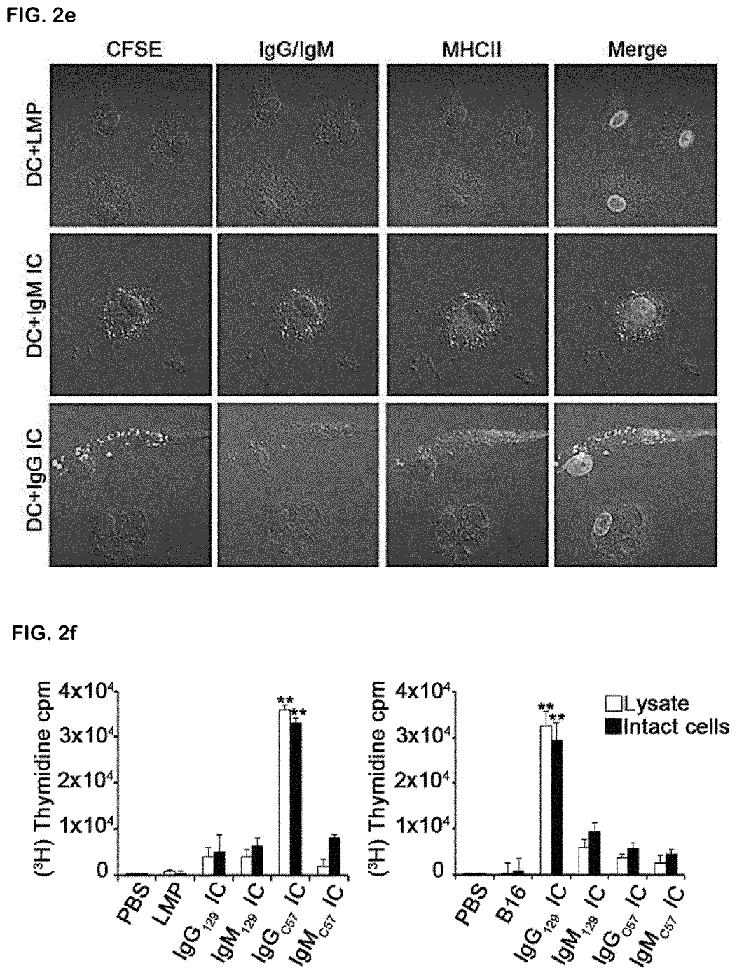

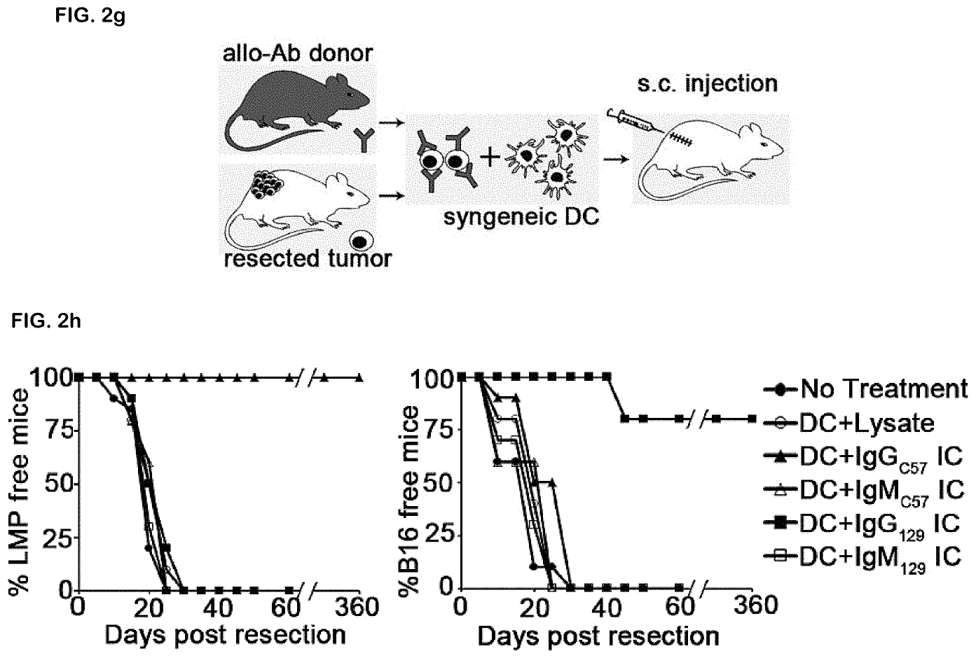

To investigate the effect of these antibodies on tumor uptake by DC, intact tumor cells or tumor lysates were incubated with syngeneic or allogeneic antibodies to form immune complexes (IC) and added these to bone marrow-derived (BM) DC (FIG. 2a). Only IC formed with allogeneic IgG antibodies (alloIgG-IC) or IgM antibodies (allogM-IC) induced BMDC activation and uptake of tumor-derived proteins (FIG. 2b-d). Confocal imaging revealed tumor proteins in close proximity to MHCII molecules (FIG. 2e), and BMDC incubated with alloIgG-IC induced significant T cell proliferation (FIG. 2f), demonstrating that tumor antigens were processed and presented.

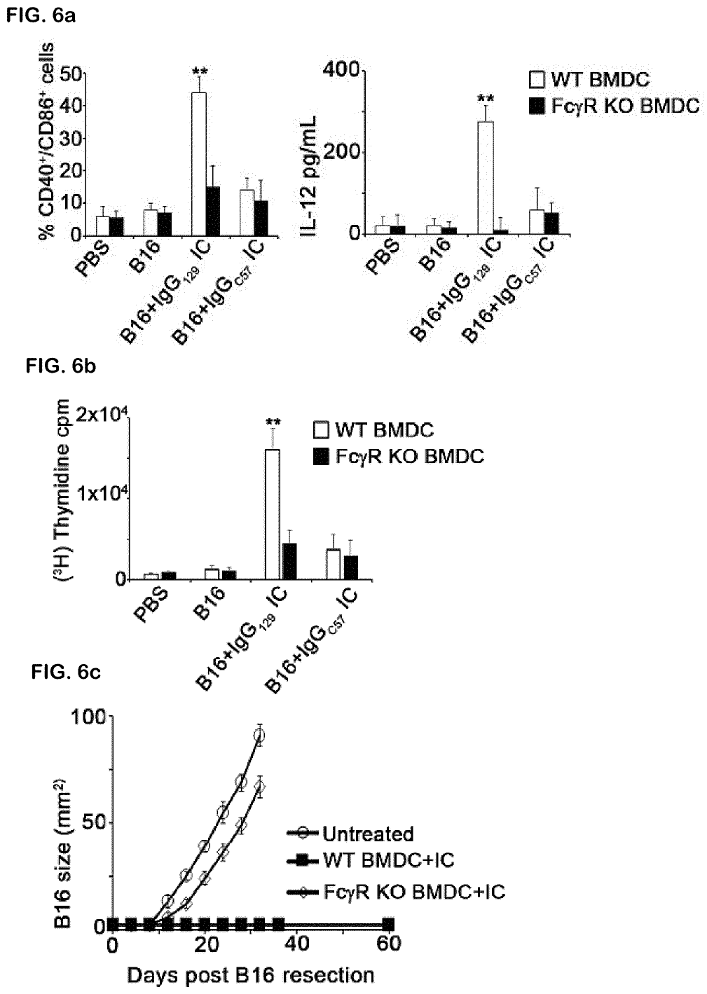

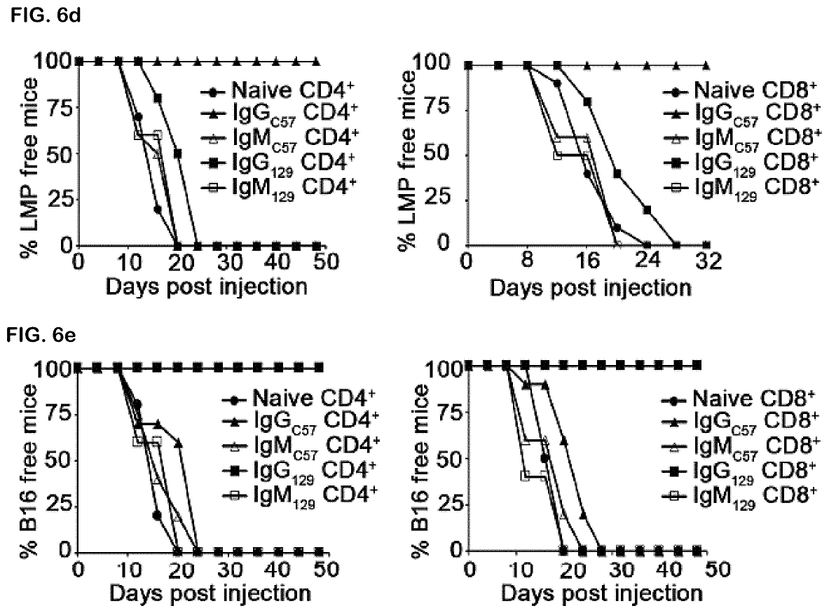

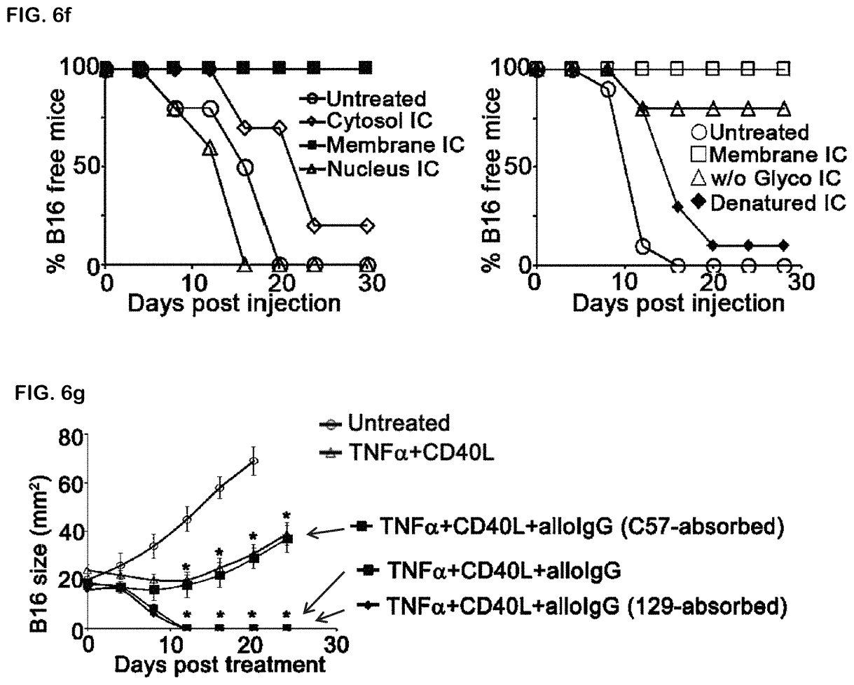

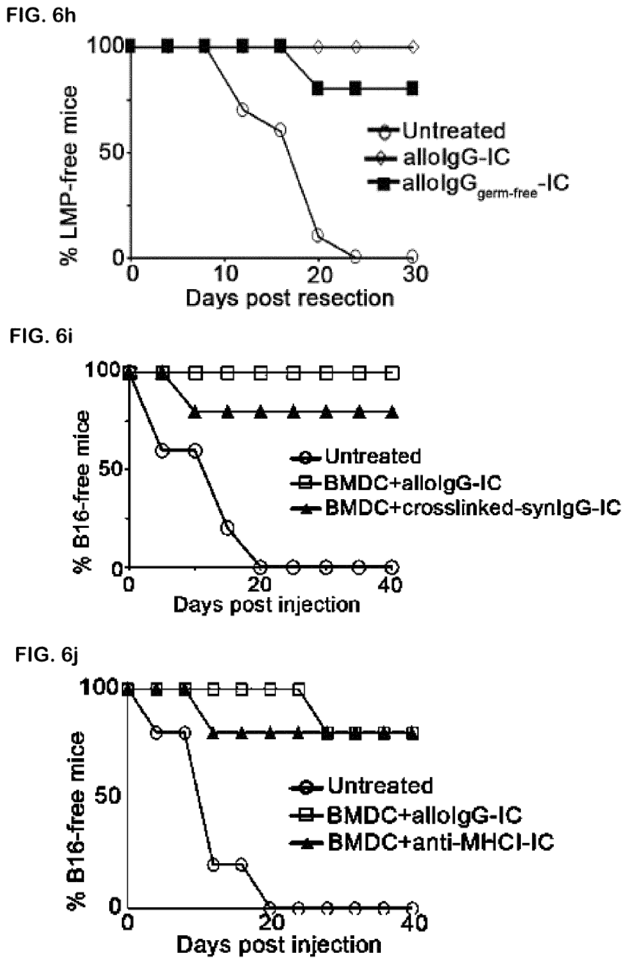

To determine whether these mechanistic principles for immune activation could elicit anti-tumor immune responses to syngeneic tumors (derived from the same mouse strain), syngeneic hosts were inoculated s.c. with B16 or LMP cells, and tumors were removed when they reached 45-55 mm², leaving macroscopic tumor-free margins of approximately 2 mm. IgG-IC or IgM-IC were prepared from excised tumors and incubated overnight with syngeneic BMDC, which were subsequently injected s.c. into the corresponding tumor-resected mouse (FIG. 2g). Nearly all mice treated with syngeneic DC loaded with alloIgG-IC remained tumor-free for at least 12 months (when experiments were terminated) (FIG. 2h). Only BMDC loaded with alloIgG-IC were sufficient to completely prevent tumor regrowth, as all other animals experienced tumor relapse within 30 days (FIG. 2h). The ability of alloIgG-IC-loaded DC to activate T cells and protect mice from tumor recurrence was completely abrogated in DC lacking FcγR (FIG. 6a-2c). Furthermore, adoptive transfer of splenic CD4⁺ or CD8′T cells from allogG-IC-treated animals into naive mice prevented growth of subcutaneous tumors (FIG. 6d-2e), demonstrating that a potent tumor-specific T cell response had been elicited.

The nature of the B16 antigens recognized by alloIgG was next investigated by modifying B16 cells or absorbing fractions of the allogG prior to IC formation and BMDC vaccination. While removing glycan residues had little effect, denaturing tumor proteins removed the therapeutic benefit (FIG. 6f). Furthermore, IC formed from membrane-bound B16 proteins prevented tumor relapse while IC formed from other subcellular protein fractions could not (FIG. 6f). Pre-absorbing alloIgG against normal skin, pancreas and spleen cells syngeneic to the tumor removed their therapeutic benefit while absorption against similar cells syngeneic to the antibodies did not (FIG. 6g). Additionally, alloIgG from germ-free mice induced tumor immunity (FIG. 6h), suggesting that IgG generated in response to microbiota was not required. These data indicate that the protective effect of alloIgG is dependent upon antibody binding to B16 membrane proteins that are likely expressed on normal cells.

The binding of antibodies, but not the identity of the antigens bound, might be essential for the induction of a tumor-eradicating immune response. Consistent with this view, IC formed by covalently crosslinking syngeneic IgG onto B16 membrane proteins still conferred a therapeutic benefit after incubation with BMDC (FIG. 6i). Moreover, IC formed using a monoclonal antibody against MHC-I, an antigen shared by the allogG donor and C57Bl/6 host, were sufficient to protect animals after incubation with BMDC (FIG. 6j). Taken together, these data demonstrate that the critical element of this therapeutic strategy is the binding of IgG to the tumor cell surface rather than the specific identity of the antigens bound or the origin of the IgG.

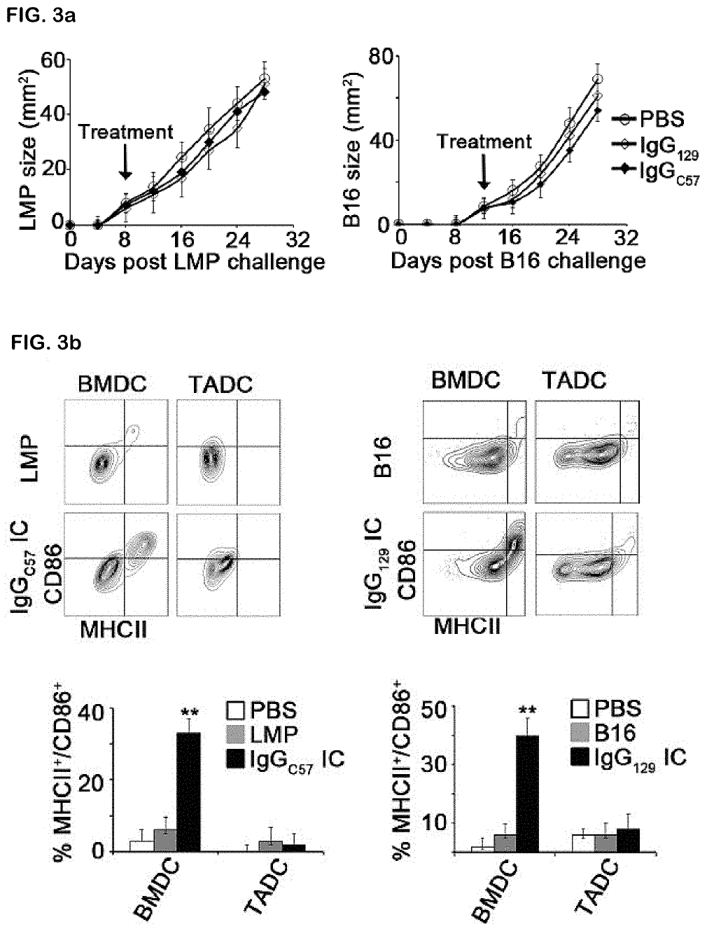

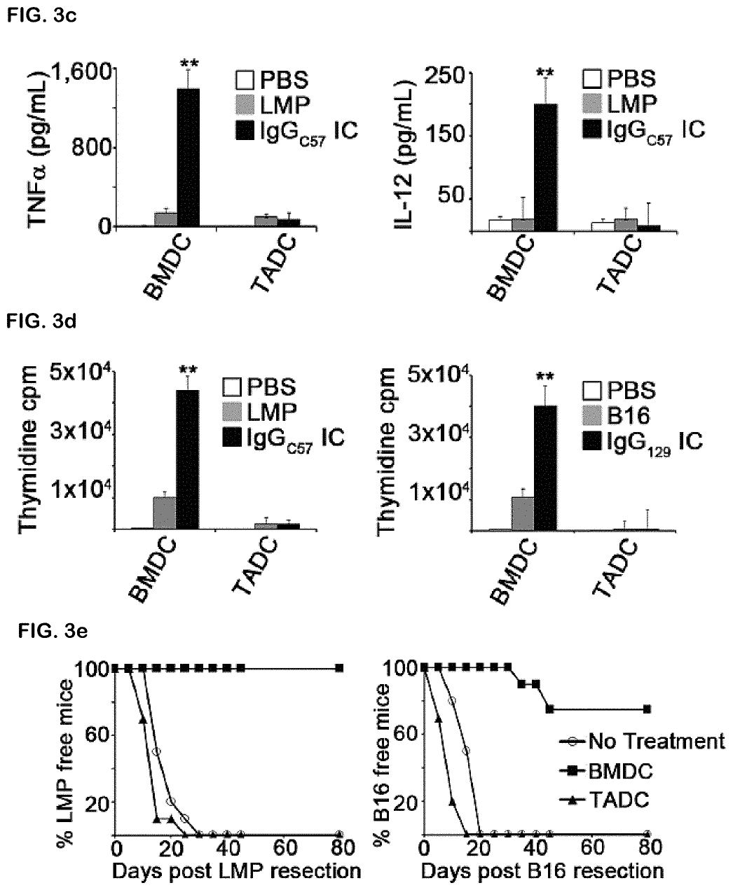

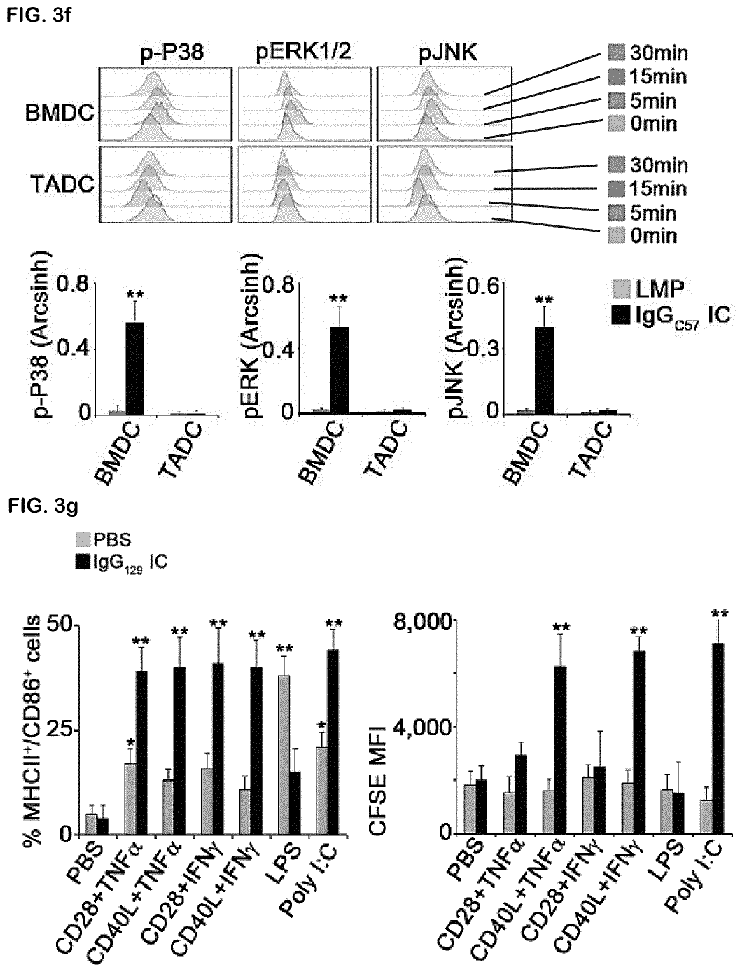

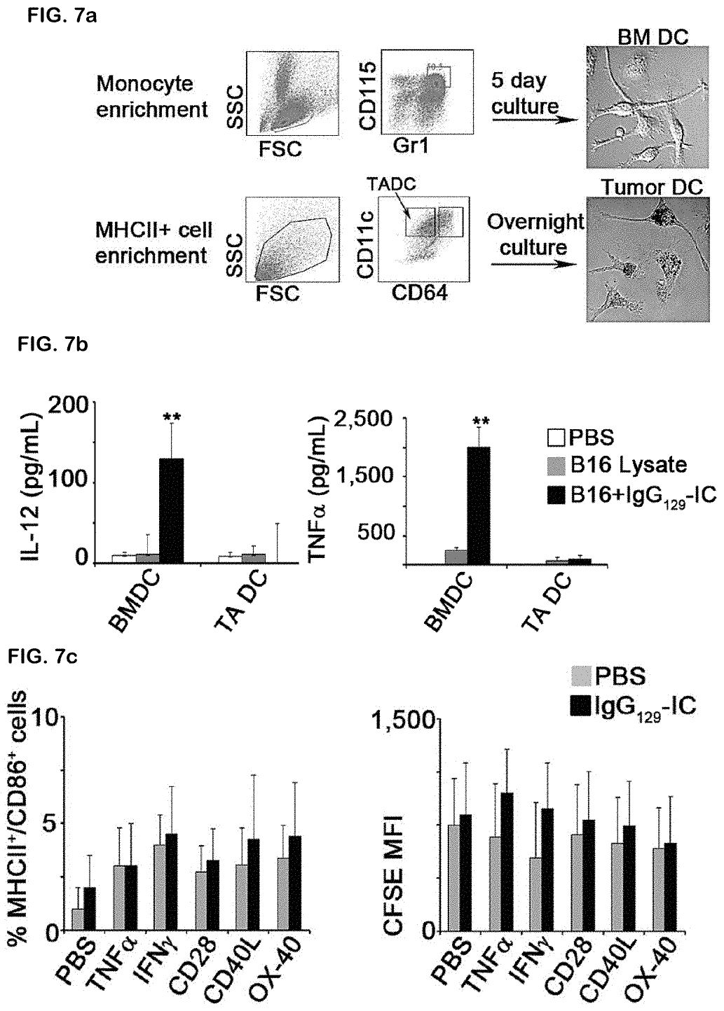

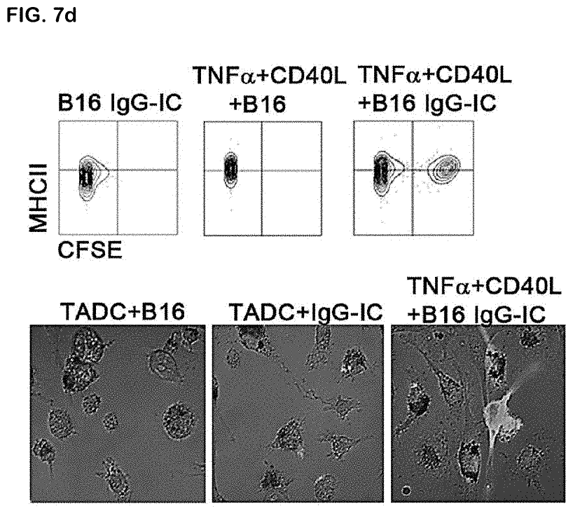

The potency of BMDC activated with alloIgG-IC suggested that direct injection of alloIgG into syngeneic tumors might also induce tumor regression. However, only minor effects were observed when alloIgG was injected into B16 or LMP tumors growing in autologous hosts (FIG. 3a). To resolve this apparent discrepancy, tumor-associated DC (TADC) (FIG. 7a) were obtained and cultured these cells with tumor lysates or alloIgG-IC. In contrast to BMDC, TADC displayed no activation (FIG. 3b-d and FIG. 7b) and had no effect on tumor recurrence (FIG. 3e). To understand why TADC failed to respond to alloIgG-IC, the cell signaling pathways known to be activated upon FcγR stimulation were investigated. Strong p38, ERK1/2 and JNK phosphorylation was observed in BMDC upon activation with alloIgG-IC. In contrast, TADC failed to exhibit phosphorylation of these MAP kinases (FIG. 3f). Since the expression pattern of Fcγ receptors on TADC was similar to that of BMDC, and several immune stimuli are known to induce MAPK activation in DC, the effect of such stimuli on the response of TADC to allogG-IC was tested. Addition of poly I:C, TNFα+CD40L, or IFNγ+CD40L enabled activation of TADC as well as uptake of alloIgG-IC (FIG. 3g and FIG. 7c-3d).

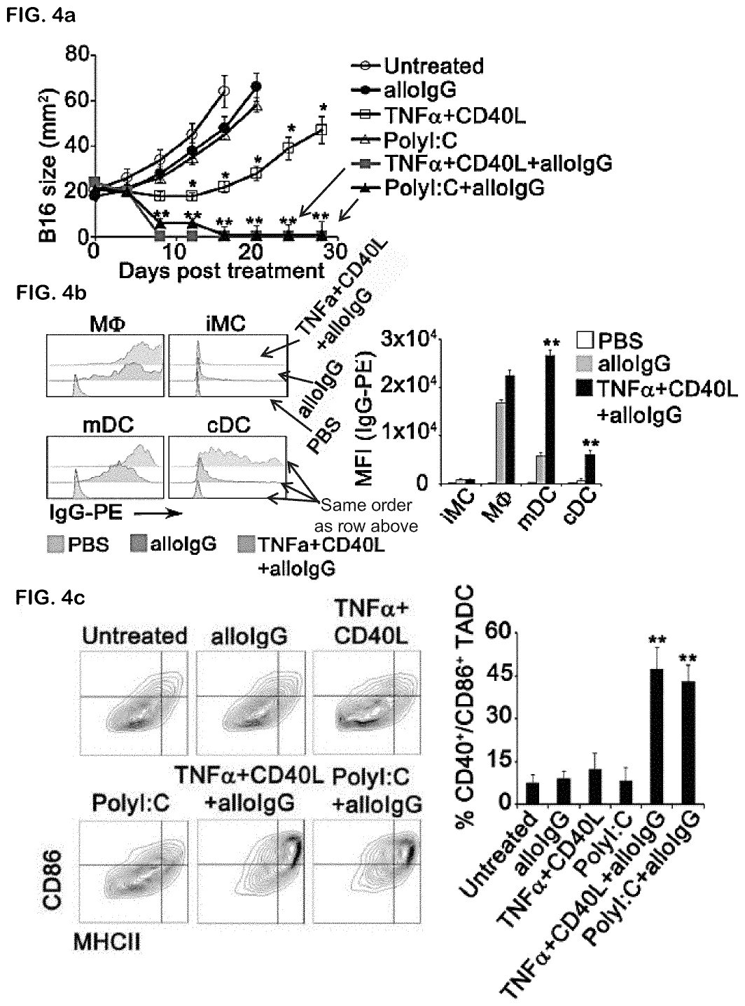

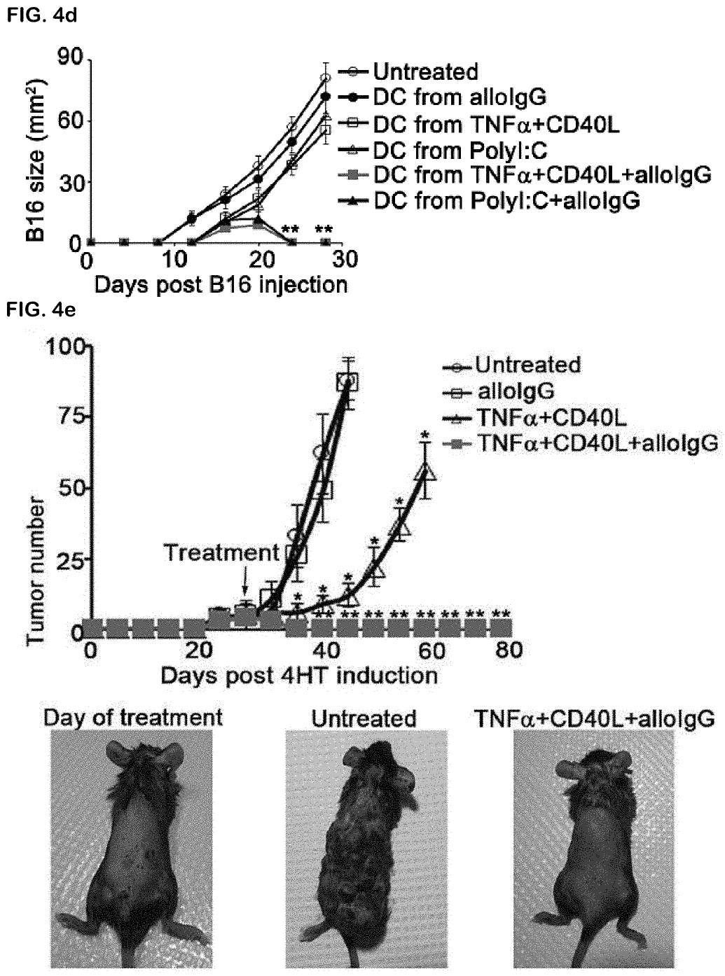

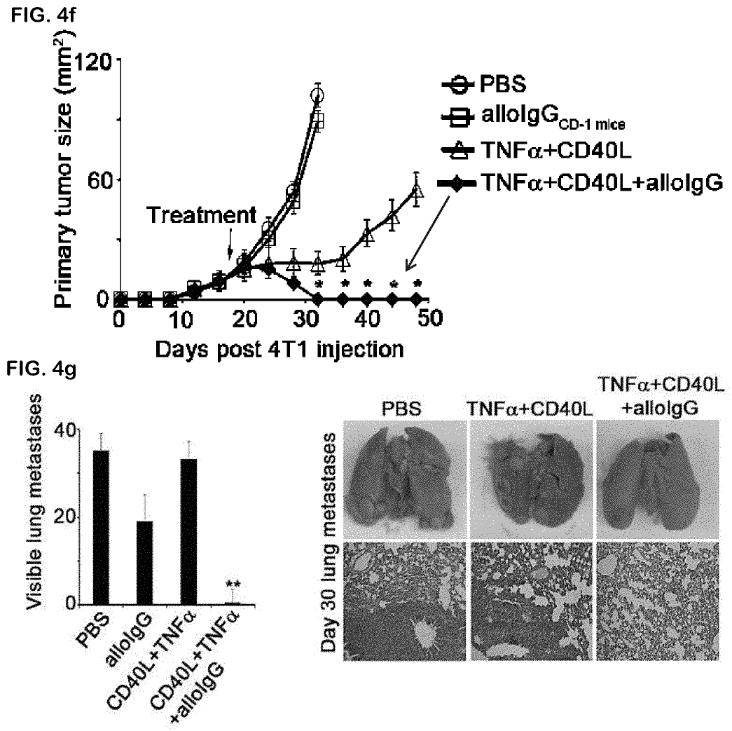

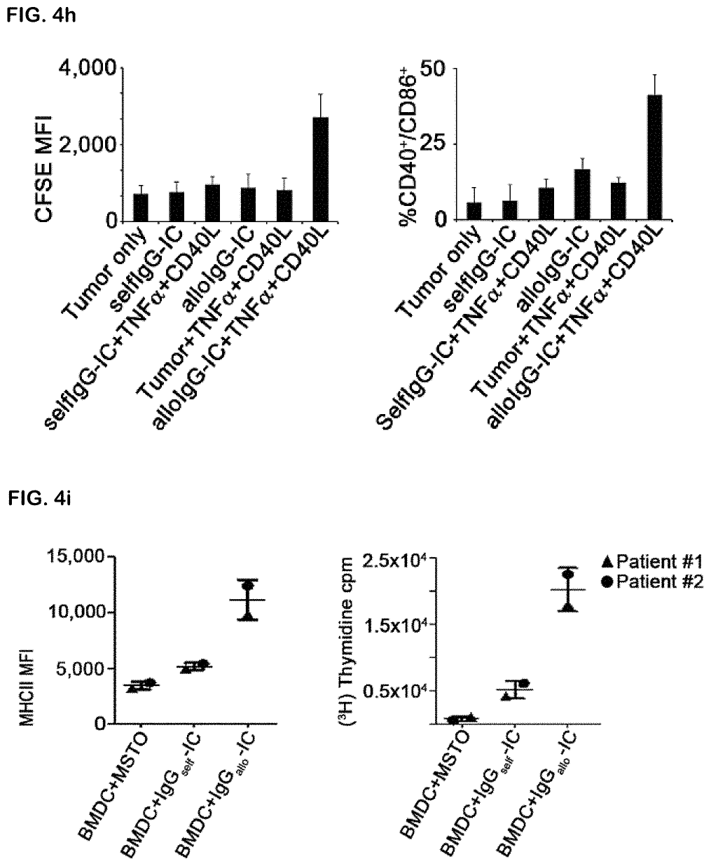

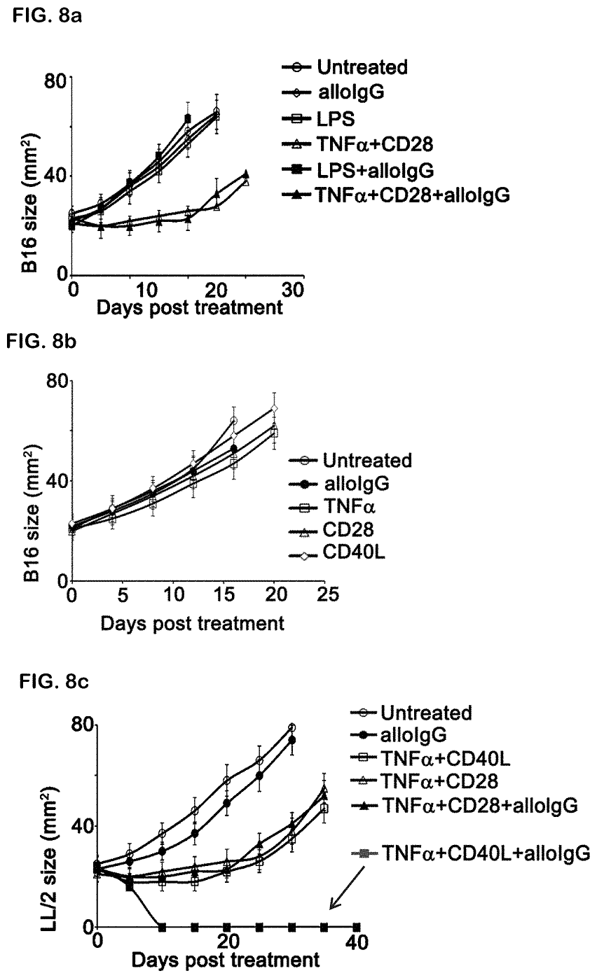

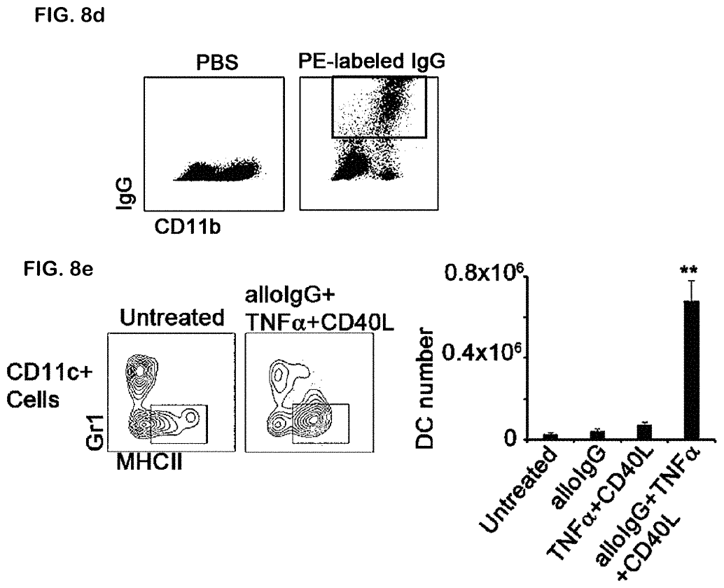

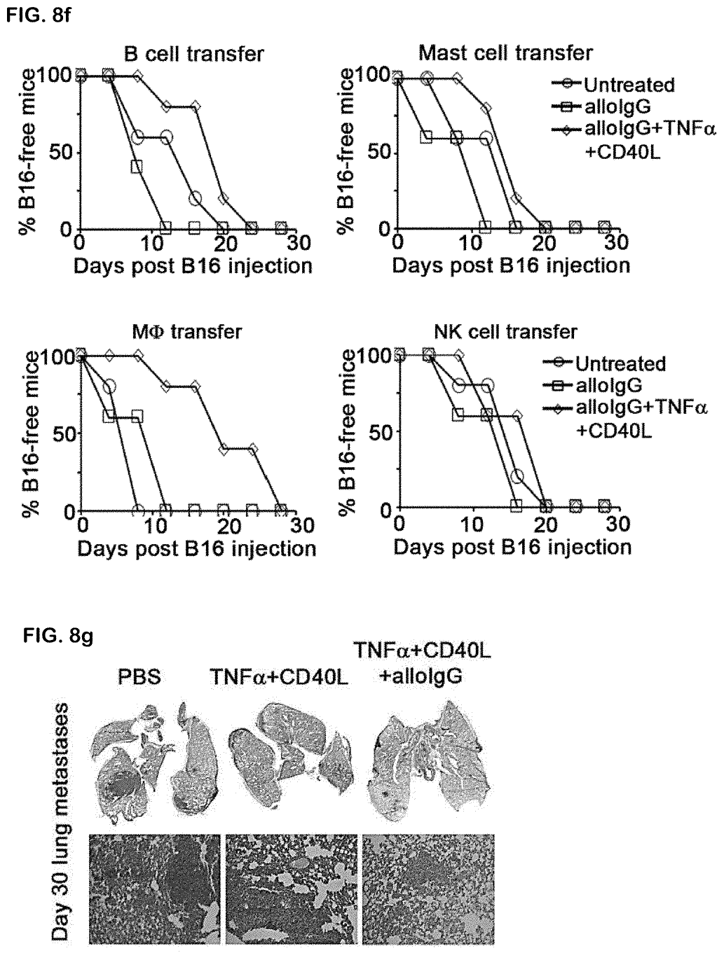

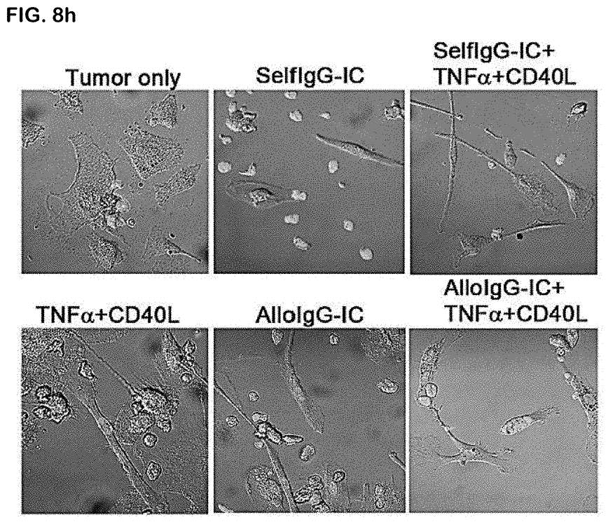

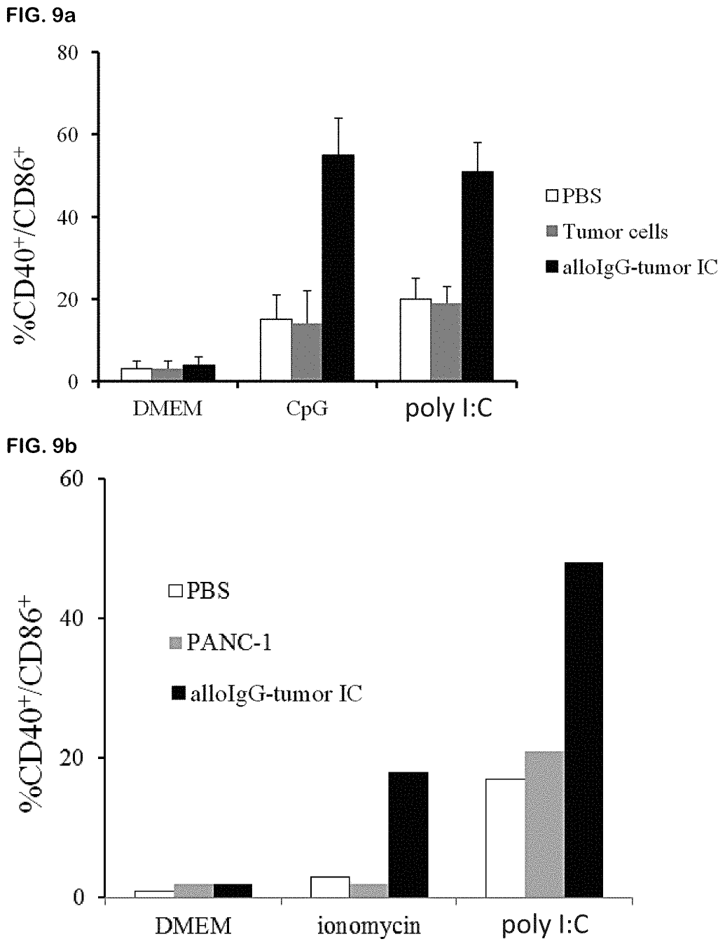

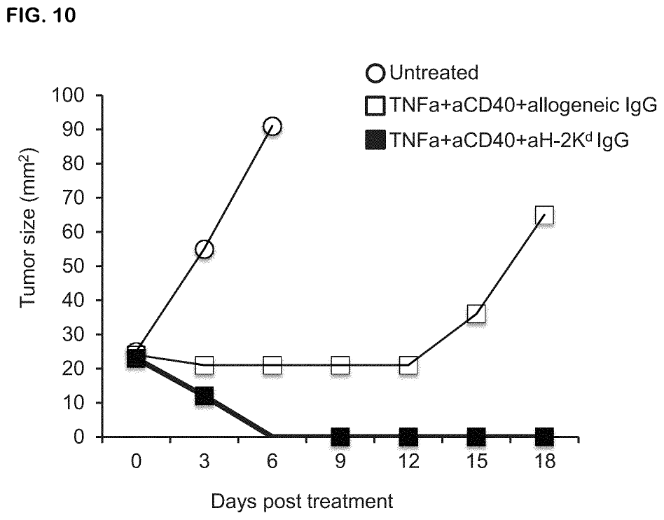

Whether alloIgG in combination with one of these stimuli could induce immune responses to syngeneic tumors in situ was subsequently tested. Naive C57B1I/6 mice were inoculated with B16 cells, and tumors were allowed to grow until they reached 18-25 mm². Intratumoral injection of alloIgG in combination with either TNFα+CD40L or poly I:C induced complete tumor elimination (FIG. 4a and FIG. 8a-b). Similar results were also obtained in mice challenged with Lewis Lung carcinoma (LL/2) (FIG. 8c).

To assess which cell types respond to IgG under these conditions, alloIgG was covalently labeled with phycoerythrin and injected intratumorally. It was possible that the cells mediating the therapeutic effects of alloIgG would exhibit greater binding to alloIgG in the presence of a productive anti-tumor immune response (alloIgG+TNFα+CD40L) than in the absence of such a response (alloIgG alone). While immature myeloid cells (SSC^{lo}/Gr1^{hi}/CD11b^{hi}) and macrophages (CD11b⁺/Gr1^{neg}/F480⁺/MHCII⁺/CD64^{hi}) bound IgG to a similar extent in both of these scenarios, only mDC (CD11b⁺/Gr1^{neg}/CD11c⁺/MHCII⁺/CD64^{dull}) and cDC (CD11b^{neg}/CD11c^{hi}/MHCII⁺) markedly increased their IgG binding during an effective anti-tumor immune response (FIG. 4b and FIG. 8d). Moreover, analysis of infiltrating immune cells from treated B16 tumors showed significant activation of DC at the tumor site (FIG. 4c) and migration of DC into the draining lymph nodes (FIG. 8e). Additionally, adoptive transfer of TADC into naive mice conferred complete protection against subsequent challenge with B16 (FIG. 4d), demonstrating that these DC were sufficient to mediate potent anti-tumor immunity. By contrast, adoptive transfer of macrophages from the same treated mice had only a modest protective effect, while B cells, NK cells and mast cells had no effect (FIG. 8f). In sum, these results point to a critical and sufficient role for DC in mediating the therapeutic effects of alloIgG antibodies.

This therapeutic strategy was next tested in an aggressive genetically-engineered mouse melanoma model driven by mutated Braf (V600E) and loss of Pten¹⁸. Twenty-eight days after tumor induction, mice were injected intratumorally with alloIgG+TNFα+CD40L. While untreated mice developed 80-155 tumors within three weeks, treated mice experienced complete responses lasting over 8 weeks not only in the injected tumors but also in distant sites (FIG. 4e). To assess whether these systemic responses were extendable to metastases, animals bearing orthotopic 4T1 breast tumors were treated on day 16 by injection of their primary tumors, and the effect on lung metastases was tested on day 30. At the time of treatment, when tumor spread into the draining lymph node and lung micrometastases are readily observed, all mice had palpable tumor-draining lymph nodes indicative of tumor spread. Only treatment with allogG+TNFα+CD40L led to almost complete resolution of visible metastases as well as primary tumors (FIG. 4f-g). Histologic analysis of the lungs indicated complete tumor regression in 40% of the mice, and the few remaining micrometastases were heavily infiltrated with leukocytes (FIG. 4g and FIG. 8g). In sum, these results demonstrate that activation of DC via tumor-binding antibodies initiates potent and systemic anti-tumor immune responses.

To assess the clinical relevance of these findings, whether alloIgG, TNFα and CD40L could induce tumor uptake and maturation of human TADC was tested. CD11c⁺/MHCII⁺ cells from the tumors of two human patients with stage I lung carcinoma were incubated with autologous tumor cells coated with selfIgG or with pooled alloIgG from ten healthy donors. Addition of TNFα+CD40L enabled these DC to internalize alloIgG-IC and concomitantly induced marked upregulation of CD40 and CD86, indicative of activation (FIG. 4h and FIG. 8h). These data suggest that the mechanism by which tumor-alloIgG IC activates DC is conserved between species. Whether DC loaded with alloIgG-IC were capable of activating a patient's own CD4⁺ T cells was then tested. BMDC from 2 human patients with malignant pleural mesothelioma were incubated with autologous tumor lysates alone, in combination with autologous IgG, or with pooled alloIgG from healthy donors. In both patients, only BMDC incubated with pooled alloIgG-IC, but not autologous IgG-IC, exhibited marked activation, upregulating HLA-DR expression and driving proliferation of CD4⁺ T cells collected from the corresponding patient (FIG. 4i).

Over the last two decades, the role of antibodies during tumor progression has been a source of controversy. The data presented herein demonstrate that while TADC are not naturally responsive to IgG-IC, addition of specific stimuli enables them to drive tumor-eradicating immunity. The data presented herein demonstrate that presentation of tumor antigens following antibody-mediated uptake by DC is sufficient to initiate potent, systemic T cell-mediated immune responses against tumors. Furthermore, this work suggests that this fundamental mechanism of immunological recognition and targeting, which prevents tumor transmission even between MHC-matched individuals, can be exploited as a powerful therapeutic strategy for cancer.

### REFERENCES

1 Coussens, L M., Zitvogel, L. & Palucka, A. K. Neutralizing tumor-promoting chronic inflammation: a magic bullet? Science 339, 286-291, doi:10.1126/science.1232227. 339/6117/286 [pii] (2013).
2 Gabrilovich, D. I., Ostrand-Rosenberg, S. & Bronte, V. Coordinated regulation of myeloid cells by tumours. Nat Rev Immunol 12, 253-268, doi:10.1038nri3175 (2012).
3 Grivennikov, S. I., Greten, F. R. & Karin, M. Immunity, inflammation, and cancer. Cell 140, 883-899, doi:10.1016j.cell.2010.01.025 (2010).
4 Hanahan, D. & Coussens, L. M. Accessories to the crime: functions of cells recruited to the tumor microenvironment. Cancer cell 21, 309-322, doi:10.1016/j.ccr.2012.02.022. S1535-6108(12)00082-7 [pii] (2012).
5 Mantovani, A. & Sica, A. Macrophages, innate immunity and cancer: balance, tolerance, and diversity. Current opinion in immunology 22, 231-237, doi:10.1016/j.coi.2010.01.009(2010).
6 Schreiber, R. D., Old, L J. & Smyth, M. J. Cancer immunoediting: integrating immunity's roles in cancer suppression and promotion. Science 331, 1565-1570, doi:10.1126/science.1203486(2011).
7 Vesely, M. D., Kershaw, M. H., Schreiber, R. D. & Smyth, M. J. Natural innate and adaptive immunity to cancer. Annual review of immunology 29, 235-271, doi:10.1146/annurev-immunol-031210-101324 (2011).
8 Manning, T. C. et al. Antigen recognition and allogeneic tumor rejection in CD8+TCR transgenic/RAG (−/−) mice. Journal of immunology 159, 4665-4675 (1997).
9 Ferrara, J., Guillen, F. J., Sleckman, B., Burakoff, S. J. & Murphy, G. F. Cutaneous acute graft-versus-host disease to minor histocompatibility antigens in a murine model: histologic analysis and correlation to clinical disease. J Invest Dermatol 86, 371-375 (1986).
10 Appelbaum, F. R. Haematopoietic cell transplantation as immunotherapy. Nature 411, 385-389, doi:10.1038/35077251 (2001).
11 Bishop, M. R. et al. Allogeneic lymphocytes induce tumor regression of advanced metastatic breast cancer. J Clin Oncol 22, 3886-3892, doi:10.1200/JCO.2004.01.127 (2004).
12 Goulmy, E. Minor histocompatibility antigens: allo target molecules for tumor-specific immunotherapy. Cancer J 10, 1-7 (2004).
13 Tseng, W. W. et al. Development of an orthotopic model of invasive pancreatic cancer in an immunocompetent murine host. Clinical cancer research: an official journal of the American Association for Cancer Research 16, 3684-3695, doi:10.1158/1078-0432.CCR-09-2384 (2010).
14 Baker, K. et al. Neonatal Fc receptor expression in dendritic cells mediates protective immunity against colorectal cancer. Immunity 39, 1095-1107, doi:10.1016/j.immuni.2013.11.003(2013).
15 Dhodapkar, K. M., Krasovsky, J., Williamson, B. & Dhodapkar, M. V. Antitumor monoclonal antibodies enhance cross-presentation of Cellular antigens and the generation of myeloma-specific killer T cells by dendritic cells. The Journal of experimental medicine 195, 125-133 (2002).
16 Rafiq, K., Bergtold, A.& Clynes, R. Immune complex-mediated antigen presentation induces tumor immunity. The Journal of clinical investigation 110, 71-79, doi:10.1172/JC15640 (2002).
17 Regnault, A. et al. Fcgamma receptor-mediated induction of dendritic cell maturation and major histocompatibility complex class I-restricted antigen presentation after immune complex internalization. The Journal of experimental medicine 189, 371-380 (1999).
18 Dankort, D. et al. Braf (V600E) cooperates with Pten loss to induce metastatic melanoma. Nat Genet 41, 544-552, doi:10.1038/ng.356 (2009).
19 Pulaski, B. A. & Ostrand-Rosenberg, S. Reduction of established spontaneous mammary carcinoma metastases following immunotherapy with major histocompatibility complex class II and B7.1 cell-based tumor vaccines. Cancer research 58, 1486-1493 (1998).
20 Qin, Z. et al. B cells inhibit induction of T cell-dependent tumor immunity. Nature medicine 4, 627-630 (1998).
21 de Visser, K. E., Korets, L. V. & Coussens, L M. De novo carcinogenesis promoted by chronic inflammation is B lymphocyte dependent. Cancer cell 7, 411-423, doi:10.1016/j.ccr.2005.04.014(2005).
22 Andreu, P. et al. FcRgamma activation regulates inflammation-associated squamous carcinogenesis. Cancer cell 17, 121-134, doi:10.1016/j.ccr.2009.12.019. S1535-6108(09)00431-0 [pi] (2010).
23 Gerber, J. S. & Mosser, D. M. Reversing lipopolysaccharide toxicity by ligating the macrophage Fc gamma receptors. Journal of immunology 166, 6861-6868 (2001).
24 Soussi, T. p53 Antibodies in the sera of patients with various types of cancer a review. Cancer research 60, 1777-1788 (2000).
25 Gumus, E. et al. Association of positive serum anti-p53 antibodies with poor prognosis in bladder cancer patients. International journal of urology: official journal of the Japanese Urological Association 11, 1070-1077, doi:10.1111/j.1442-2042.2004.00948.x (2004).
26 Li, Q. et a. Adoptive transfer of tumor reactive B cells confers host T-cell immunity and tumor regression. Clinical cancer research: an official journal of the American Association for Cancer Research 17, 4987-4995, doi:10.1158/1078-0432.CCR-11-0207 (2011).
27 DiLillo, D. J., Yanaba, K. & Tedder, T. F. B cells are required for optimal CD4+ and CD8+ T cell tumor immunity: therapeutic B cell depletion enhances B16 melanoma growth in mice. Journal of immunology 184, 4006-4016, doi:10.4049jimmunol.0903009 (2010).
28 Clynes, R., Takechi, Y., Moroi, Y., Houghton, A. & Ravetch, J. V. Fc receptors are required in passive and active immunity to melanoma. Proceedings of the National Academy of Sciences of the United States of America 95, 652-656 (1998).
29 Nimmerjahn, F. & Ravetch, J. V. Divergent immunoglobulin g subclass activity through selective Fc receptor binding. Science 310, 1510-1512, doi:10.1126/science.1118948 (2005).
30 Hamanaka, Y. et al. Circulating anti-MUC1 IgG antibodies as a favorable prognostic factor for pancreatic cancer. International journal of cancer. Journal international du cancer 103, 97-100, doi:10.1002/ijc.10801 (2003).
31 Kurtenkov, O. et al. Humoral immune response to MUC1 and to the Thomsen-Friedenreich (TF) glycotope in patients with gastric cancer relation to survival. Acta Oncol 46, 316-323, doi:10.1080/02841860601055441 (2007).

### Example 2: AllolaG Antibodies can Recognize Antigens that are not Typically Recognized by Syngeneic IgG

Immunoprecipitation and mass spectroscopy was used to identify antigens in B16 melanoma (mouse tumor) recognized by allogeneic IgG (“alloIgG”)(from sera of 129 mice) vs. syngeneic IgG (“syngG”)(from sera of C57Bl/6 mice). SynIgG precipitated 11 proteins that were not also pulled down by alloIgG (Table 1). To the contrary, alloIgG precipitated many proteins not recognized by synIgG (Table 2). Protein precipitated by both synIgG and alloIgG are presented in Table 3. Thus antibodies that target any one of the proteins in Table 2 (e.g., or their orthologs, such as their human orthologs) can be used as a suitable allogeneic IgG antibody in the subject methods, compositions, and kits (e.g., to induce an anti-tumor effect when used in combination with DC stimulation).

TABLE 1
Proteins enriched by synIgG (Proteins precipitated by synIgG, but not by alloIgG)

| | | |
|---|---|---|
| Identified Proteins | Accession | syn/allo ratio |

Mitochondrial membrane

| | | | |
|---|---|---|---|
| 1 | Mitochondrial inner membrane | sp\|Q8CAQ8\|IMMT_MOUSE | 2.99 |
| | protein | | |
| 2 | Trifunctional enzyme subunit | sp\|Q99JY0\|ECHB_MOUSE | 2.60 |
| | beta, mitochondrial | | |
| 3 | CDGSH iron-sulfur domain- | sp\|Q91WS0\|CISD1_MOUSE | 2 |
| | containing protein 1 | | |
| 4 | Arginine and glutamate-rich | sp\|Q3UL36\|ARGL1_MOUSE | 2 |
| | protein 1 | | |

Endoplasmic Reticulum membrane

| | | | |
|---|---|---|---|
| 1 | Stromal cell-derived factor 2-like | sp\|Q9ESP1\|SDF2L_MOUSE | 3 |
| | protein 1 | | |
| 2 | Nicalin | sp\|Q8VCM8\|NCLN_MOUSE | 2 |
| 3 | Translocation protein SEC62 | sp\|Q8BU14\|SEC62_MOUSE | 4 |
| 4 | Disco-interacting protein 2 | sp\|Q3UH60\|DIP2B_MOUSE | 4 |
| | homolog B | | |

Melanosomes and Vesicles membranes

| | | | |
|---|---|---|---|
| 1 | Vacuolar protein sorting- | sp\|Q9EQH3\|VPS35_MOUSE | 2 |
| | associated protein 35 | | |
| 2 | Angiomotin-like protein 2 | sp\|Q8K371\|AMOL2_MOUSE | 6 |
| 3 | Fibrous sheath-interacting | sp\|A2ARZ3\|FSIP2_MOUSE | 9 |
| | protein 2 | | |

TABLE 2
Proteins enriched by alloIgG (Proteins precipitated by alloIgG, but not by synIgG)

| | | |
|---|---|---|
| Identified Proteins | Accession | allo/syn ratio |

Mitochondrial membrane

| | | | |
|---|---|---|---|
| 1 | ATP synthase subunit e, | sp\|Q06185\|ATP5I_MOUSE | 2 |
| | mitochondrial | | |
| 2 | Ornithine aminotransferase, | sp\|P29758\|OAT_MOUSE | 2 |
| | mitochondrial | | |
| 3 | Apoptosis-inducing factor 1, | sp\|Q9Z0X1\|AIFM1_MOUSE | 3 |
| | mitochondrial | | |
| 4 | Amine oxidase [flavin-containing] | sp\|Q64133\|AOFA_MOUSE | 4 |
| | A | | |
| 5 | Bifunctional | sp\|P18155\|MTDC_MOUSE | 1.67 |
| | methylenetetrahydrofolate | | |
| | dehydrogenase/cyclohydrolase, | | |
| | mitochondrial | | |
| 6 | Calcium-binding mitochondrial | sp\|Q8BH59\|CMCl_MOUSE | 5 |
| | carrier protein Aralar1 | | |
| 7 | Presequence protease, | sp\|Q8K411\|PREP_MOUSE | 5 |
| | mitochondrial | | |
| 8 | ATP-dependent zinc | sp\|O88967\|YMEL1_MOUSE | 1.67 |
| | metalloprotease YME1L1 | | |
| 9 | Leucine-rich PPR motif- | sp\|Q6PB66\|LPPRC_MOUSE | 1.67 |
| | containing protein, mitochondrial | | |
| 10 | Lon protease homolog, | sp\|Q8CGK3\|LONM_MOUSE | 6 |
| | mitochondrial | | |
| 11 | Aconitate hydratase, | sp\|Q99KI0\|ACON_MOUSE | 6 |
| | mitochondrial | | |
| 12 | 2-oxoglutarate dehydrogenase, | sp\|Q60597\|ODO1_MOUSE | 7 |
| | mitochondrial | | |
| 13 | Isocitrate dehydrogenase | sp\|P54071\|IDHP_MOUSE | 2.92 |
| | [NADP], mitochondrial | | |
| 14 | Aldehyde dehydrogenase, | sp\|P47738\|ALDH2_MOUSE | 3.34 |
| | mitochondrial | | |
| 15 | ATP synthase subunit beta, | sp\|P56480\|ATPB_MOUSE | 3.13 |
| | mitochondrial | | |
| 16 | Aspartate aminotransferase, | sp\|P05202\|AATM_MOUSE | 11 |
| | mitochondrial | | |

Endoplasmic Reticulum membrane

| | | | |
|---|---|---|---|
| 1 | Transmembrane protein 93 | sp\|Q9CQW0\|TMM93_MOUSE | 2 |
| 2 | Endoplasmic reticulum-Golgi | sp\|Q9CQE7\|ERGI3_MOUSE | 2 |
| | intermediate compartment | | |
| | protein 3 | | |
| 3 | Reticulon-4 | sp\|Q99P72\|RTN4_MOUSE | 2 |
| 4 | Uncharacterized protein | sp\|Q8BQR4\|CL041_MOUSE | 2 |
| | C12orf41 homolog | | |
| 5 | Erlin-2 | sp\|Q8BFZ9\|ERLN2_MOUSE | 2 |
| | | (+1) | |
| 6 | Transitional endoplasmic | sp\|Q01853\|TERA_MOUSE | 2 |
| | reticulum ATPase | | |
| 7 | Dolichyl- | sp\|P61804\|DAD1_MOUSE | 2 |
| | diphosphooligosaccharide— | | |
| | protein glycosyltransferase | | |
| | subunit DAD1 | | |
| 8 | Calnexin | sp\|P35564\|CALX_MOUSE | 2 |
| 9 | Calumenin | sp\|O35887\|CALU_MOUSE | 2 |
| 10 | Vesicle-associated membrane | sp\|Q9WV55\|VAPA_MOUSE | 3 |
| | protein-associated protein A | | |
| 11 | Mannosyl-oligosaccharide | sp\|Q80UM7\|MOGS_MOUSE | 3 |
| | glucosidase | | |
| 12 | Neutral alpha-glucosidase | sp\|Q8BHN3\|GANAB_MOUSE | 3 |
| 13 | ERO1-like protein alpha | sp\|Q8R180\|ERO1A_MOUSE | 5 |
| 14 | UDP-glucose: glycoprotein | sp\|Q6P5E4\|UGGG1_MOUSE | 5 |
| | glucosyltransferase 1 | | |
| 15 | Prolyl 4-hydroxylase subunit | sp\|Q60715\|P4HA1_MOUSE | 5 |
| | alpha-1 | | |
| 16 | Epoxide hydrolase 1 | sp\|Q9D379\|HYEP_MOUSE | 9 |
| 17 | Calreticulin | sp\|P14211\|CALR_MOUSE | 14 |
| 18 | Sarcoplasmic/endoplasmic | sp\|O55143\|AT2A2_MOUSE | 1.88 |
| | reticulum calcium ATPase | | |
| 19 | Protein disulfide-isomerase A4 | sp\|P08003\|PDIA4_MOUSE | 12 |
| 20 | Protein disulfide-isomerase | sp\|P09103\|PDIA1_MOUSE | 12 |
| 21 | Protein disulfide-isomerase A3 | sp\|P27773\|PDIA3_MOUSE | 9 |
| 22 | Protein disulfide-isomerase A6 | sp\|Q922R8\|PDIA6_MOUSE | 11 |

Melanosomes and Vesicles membranes

| | | | |
|---|---|---|---|
| 1 | Clathrin heavy chain 1 | sp\|Q68FD5\|CLH_MOUSE | 5 |
| 2 | Peptidyl-prolyl cis-trans | sp\|P24369\|PPIB_MOUSE | 7 |
| | isomerase B | | |

Cell membrane

| | | | |
|---|---|---|---|
| 1 | T-complex protein 1 subunit | sp\|P80318\|TCPG_MOUSE | 2 |
| | gamma | | |
| 2 | Monocarboxylate transporter 4 | sp\|P57787\|MOT4_MOUSE | 2 |
| 3 | Nicastrin | sp\|P57716\|NICA_MOUSE | 2 |
| 4 | Basigin | sp\|P18572\|BASI_MOUSE | 2 |
| 5 | Vesicle-associated membrane | sp\|Q9WV55\|VAPA_MOUSE | 3 |
| | protein-associated protein A | | |
| 6 | Retrovirus-related Env | sp\|P11370\|ENV2_MOUSE | 3 |
| | polyprotein from Fv-4 | | |
| 7 | Synaptic vesicle membrane | sp\|Q62465\|VAT1_MOUSE | 4 |
| | protein | | |
| 8 | 4F2 cell-surface antigen heavy | sp\|P10852\|4F2_MOUSE | 4 |
| | chain | | |
| 9 | Alpha-enolase | sp\|P17182\|ENOA_MOUSE | 5 |
| 10 | lntegrin-linked protein kinase | sp\|O55222\|ILK_MOUSE | 4 |
| 11 | Transmembrane glycoprotein | sp\|Q99P91\|GPNMB_MOUSE | 6.26 |
| | NMB | | |
| 12 | MLV-related proviral Env | sp\|P10404\|ENV1_MOUSE | 13 |
| | polyprotein | | |
| 13 | ERO1-like protein alpha | sp\|Q8R180\|ERO1A_MOUSE | 5 |
| 14 | Clathrin heavy chain 1 | sp\|Q68FD5\|CLH_MOUSE | 5 |
| 15 | Desmoglein-1-alpha | sp\|Q61495\|DSG1A_MOUSE | 2.09 |
| | | (+2) | |
| 16 | Sodium/potassium-transporting | sp\|Q8VDN2\|AT1A1_MOUSE | 2.50 |
| | ATPase subunit alpha-1 | | |

Heat shock and stress proteins

| | | | |
|---|---|---|---|
| 1 | Hypoxia up-regulated protein 1 | sp\|Q9JKR6\|HYOU1_MOUSE | 2 |
| 2 | Heat shock protein 75 kDa, | sp\|Q9CQN1\|TRAP1_MOUSE | 3 |
| | mitochondrial | | |
| 3 | Stress-70 protein, mitochondrial | sp\|P38647\|GRP75_MOUSE | 3.41 |
| 4 | Endoplasmin HSP90 | sp\|P08113\|ENPL_MOUSE | 2.73 |
| 5 | 60 kDa heat shock protein, | sp\|P63038\|CH60_MOUSE | 2.09 |
| | mitochondrial | | |
| 6 | 10 kDa heat shock protein, | sp\|Q64433\|CH1O_MOUSE | 2.34 |
| | mitochondrial | | |

TABLE 3
Proteins not enriched (Proteins equally precipitated by alloIgG and synIgG)

| | | |
|---|---|---|
| Identified Proteins | Accession | allo/syn ratio |

Mitochondrial membrane

| | | | |
|---|---|---|---|
| 1 | Phosphate carrier protein, | sp\|Q8VEM8\|MPCP_MOUSE | 0.60 |
| | mitochondrial | | |
| 2 | Succinate dehydrogenase | sp\|Q8K2B3\|DHSA_MOUSE | 1.34 |
| | [ubiquinone] flavoprotein | | |
| | subunit, mitochondrial | | |
| 3 | Calcium-binding mitochondrial | sp\|Q9QXX4\|CMC2_MOUSE | 1.11 |
| | carrier protein Aralar2 | | |
| 4 | ADP/ATP translocase 2 | sp\|P51881\|ADT2_MOUSE | 0.86 |
| 5 | ADP/ATP translocase 1 | sp\|P48962\|ADT1_MOUSE | 0.65 |
| 6 | ATP synthase subunit alpha, | sp\|Q03265\|ATPA_MOUSE | 0.96 |
| | mitochondrial | | |
| 7 | Dolichyl- | sp\|Q91YQ5\|RPN1_MOUSE | 1.25 |
| | diphosphooligosaccharide— | | |
| | protein glycosyltransferase | | |
| | subunit 1 | | |
| 8 | Elongation factor Tu, | sp\|Q8BFR5\|EFTU_MOUSE | 0.83 |
| | mitochondrial | | |
| 9 | Isocitrate dehydrogenase [NAD] | sp\|Q9D6R2\|IDH3A_MOUSE | 0.83 |
| | subunit alpha, mitochondrial | | |
| 10 | Monofunctional C1- | sp\|Q3V3R1\|C1TM_MOUSE | 1.19 |
| | tetrahydrofolate synthase, | | |
| | mitochondrial | | |
| 11 | Peroxiredoxin-1 | sp\|P35700\|PRDX1_MOUSE | 1.39 |

Endoplasmic Reticulum membrane

| | | | |
|---|---|---|---|
| 1 | DnaJ homolog subfamily B | sp\|Q99KV1\|DJB11_MOUSE | 0.63 |
| | member 11 | | |
| 2 | 78 kDa glucose-regulated | sp\|P20029\|GRP78_MOUSE | 0.81 |
| | protein | | |
| 3 | Serpin H1 | sp\|P19324\|SERPH_MOUSE | 1.29 |
| 4 | Protein transport protein Sec61 | sp\|Q9CQS8\|SC61B_MOUSE | 1.25 |
| | subunit beta | | |
| 5 | Leucine-rich repeat-containing | sp\|Q922Q8\|LRC59_MOUSE | 0.56 |
| | protein 59 | | |
| 6 | Protein transport protein Sec61 | sp\|P61620\|S61A1_MOUSE | 0.93 |
| | subunit alpha isoform 1 | | |
| 7 | Dolichyl- | sp\|O54734\|OST48_MOUSE | 1.39 |
| | diphosphooligosaccharide— | | |
| | protein glycosyltransferase 48 | | |
| | kDa subunit | | |
| 8 | Estradiol 17-beta- | sp\|O70503\|DHB12_MOUSE | 0.72 |
| | dehydrogenase 12 | | |

Melanosomes and Vesicles membranes

| | | | |
|---|---|---|---|
| 1 | Flotillin-2 | sp\|Q60634\|FLOT2_MOUSE | 0.56 |
| 2 | Cathepsin D | sp\|P18242\|CATD_MOUSE | 1.39 |
| 3 | AP-2 complex subunit beta | sp\|Q9DBG3\|AP2B1_MOUSE | 0.52 |
| 4 | AP-2 complex subunit mu | sp\|P84091\|AP2M1_MOUSE | 1.04 |
| 5 | Annexin A2 | sp\|P07356\|ANXA2_MOUSE | 1.25 |
| 6 | Melanocyte protein PMEL | sp\|Q60696\|PMEL_MOUSE | 0.67 |

Cell membrane

| | | | |
|---|---|---|---|
| 1 | Desmoplakin | sp\|E9Q557\|DESP_MOUSE | 0.80 |
| 2 | PDZ domain | sp\|Q9Z0G0\|GIPC1_MOUSE | 0.58 |
| 3 | Junction plakoglobin | sp\|Q02257\|PLAK_MOUSE | 1.11 |

### Example 3

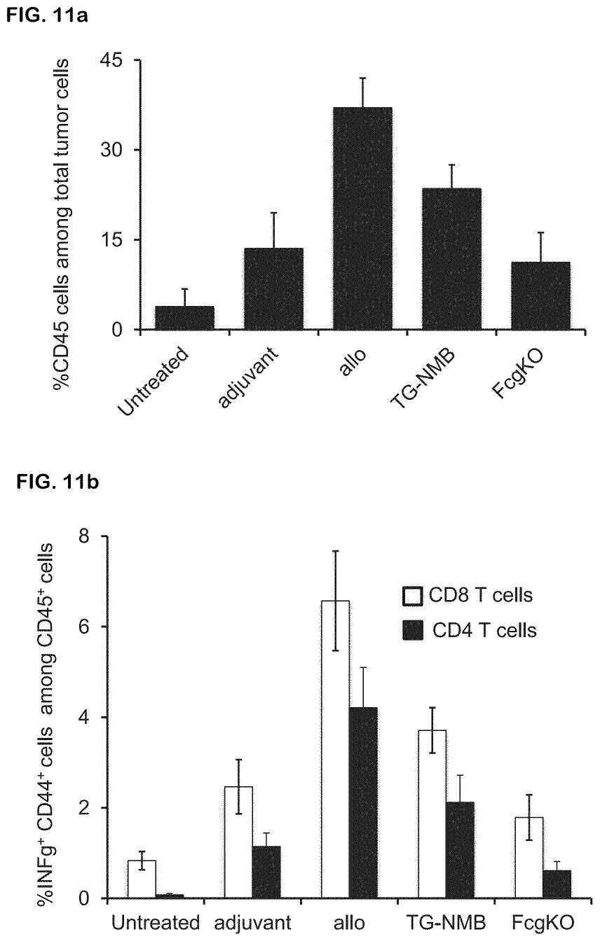

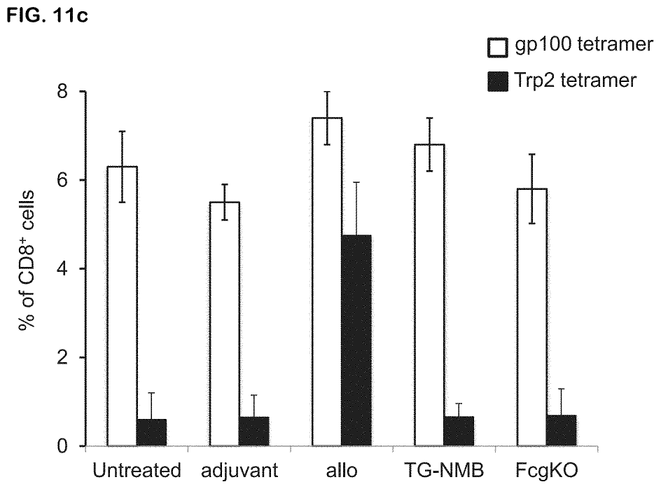

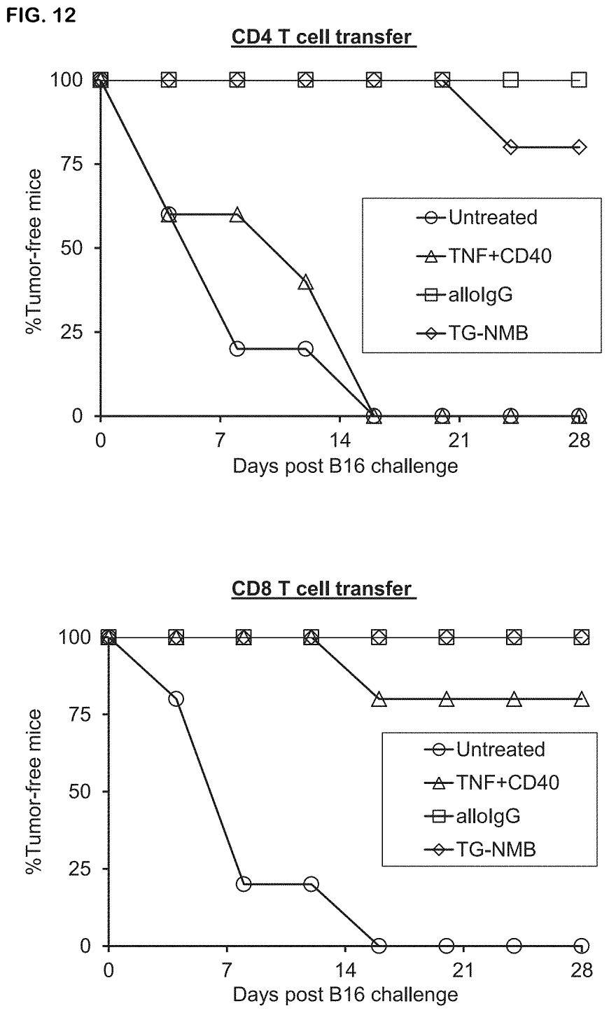

The following experimental methods and results provide evidence supporting the notion that the T cells recognize antigens (presented to them by loaded APC) that are different from those recognized by the alloantibodies that were used to load the APC. The results (FIGS. 11-14) show that the therapy induces a massive tumor infiltrate of CD45+cells (ie, mononuclear leukocytes) of which a large portion are activated CD4 and CD8 T cells. They also show that there is a significant immune response seen at sites distant from the tumor (eg., spleen) as indicated by the ability of CD4 or CD8 T cells from the spleen to protect naïve mice from tumor challenge. The results also show that the response is greater with allogG+DC stimuli than with a polyclonal antibody to a single tumor associated antigen (Transmembrane Glycoprotein-MMB) or with DC stimuli alone.

FIG. 10 illustrates that treatment of a colorectal cancer (CT26), which had been injected and grown subcutaneously, with a monoclonal mouse anti-mouse MHC class I antibody in combination with DC stimuli, resulted in complete tumor regression. Since MHC I is highly expressed on CT26 tumor cells, this result is consistent with the hypothesis that the overall amount of antibody bound to tumor cells is a determinant of the potency of the anti-tumor response. There was no systemic toxicity, although there was a significant inflammatory reaction in the vicinity of the tumor that healed completely within a few days. MHC class I is down regulated on many tumors, rendering them resistant to CD8 T cell mediated cytotoxicity. It is likely that the DC stimuli upregulated MHC I (and/or II) expression on tumors by activating T cells and perhaps other cells that infiltrated the tumor, which then secreted IFNg. In some cases, IFNg itself can be used as an APC (e.g., DC) stimulatory agent. In some cases, an anti-MHC-I antibody (e.g., combined with one or more APC stimuli, e.g., one or more DC stimuli) can have a powerful therapeutic effect on tumors that lack high level expression of MHC-I.

FIG. 10. Monoclonal allogeneic anti-MHC I antibody in combination with DC stimuli induces complete tumor regression. 4×10⁶ C.T26 colon cancer cells were injected s.c. into Balb/c mice above the right flank. Once tumors reached 25 mm², they were left untreated (open circles), injected intratumorally with TNFα+aCD40 agonist+allogeneic IgG (open squares), or with TNFα+aCD40 agonist+aH-2K^{d} IgG (an anti-MHC class I antibody)(solid squares).

FIGS. 11a-c. Immune cell infiltrate in tumors following therapy. Mice were injected s.c. with 2×10⁵ B16 melanoma cells which were allowed to grow until tumors reached 25 mm². Mice were then injected intratumorally with PBS (untreated), with TNFα+aCD40 alone, or with the combination of TNFα+aCD40+allogeneic IgG (from 129S1 mice), or TNFα+aCD40+antibody to Transmembrane Glycoprotein-NMB (TG-NMB, GPNMB). In some cases, mice lacking functional Fcg receptor signaling were injected with TNFα+aCD40+allogeneic IgG. After 6 days, tumors were excised and the entire cellular composition, including tumor cells, was tested by flow cytometry (n=8). a. Y axis is % CD45 cells among total tumor cells. b. Y axis is % INFg⁺ CD44⁺ cells among CD45⁺ cells (quantified for CD8 T cells and for CD4 T cells). c. Y axis is % of CD8⁺ cells expressing gp100 tetramer and % of CD8⁺ cells expressing Trp2 tetramer.

FIG. 12. Effect of adoptive transfer of T cells from treated mice on tumor development in naive mice. Splenic T cells were purified from B16-bearing mice, 6 days following their treatment with PBS (untreated), with TNFα+aCD40, or TNFα+aCD40 in combination with allogeneic IgG (alloIgG) or in combination with antibody to Transmembraine Glycoprotein-NMB (TG-NMB; GPNMB). 5×10⁸ CD4⁴ cells (Top) or CD8⁺ cells (Bottom) were injected i.v. into nave mice followed 1 hour later by s.c injection of 2.5×10⁵ B16 cells.

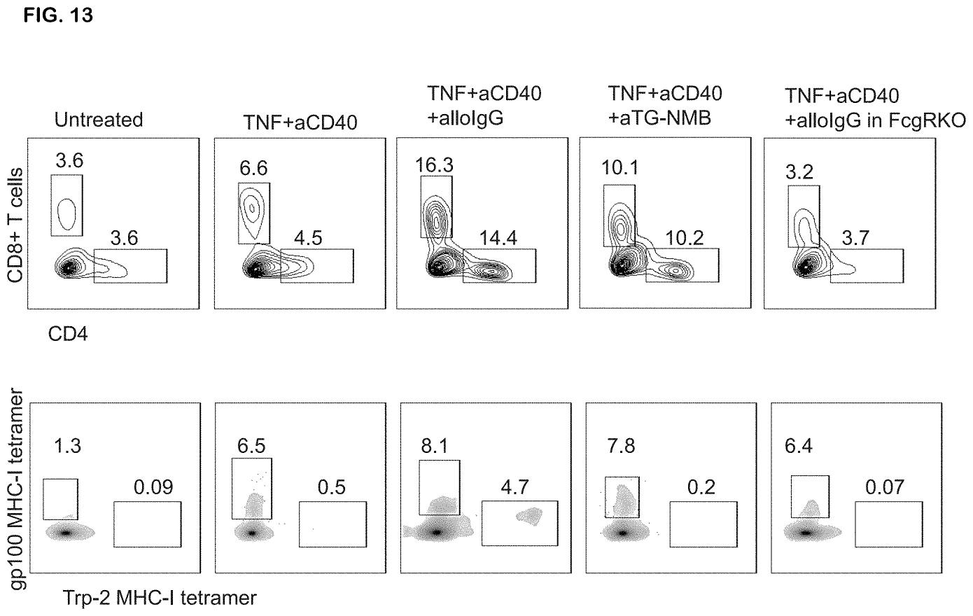

FIG. 13. Representative FACS plots from B16 tumors 6 days after treatment. Numbers represent % of positive cells.

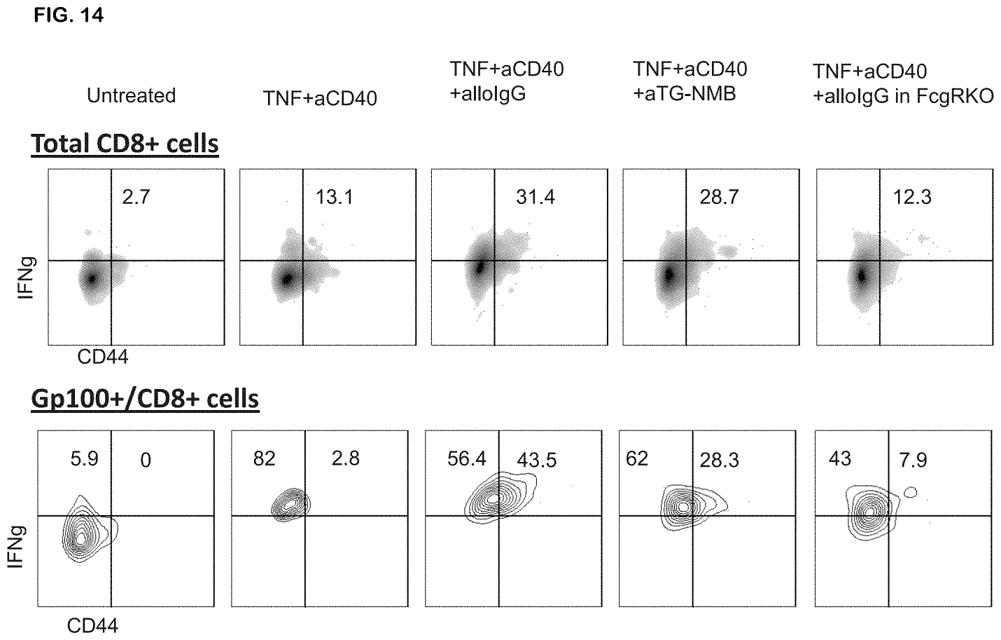

FIG. 14. Representative FACS plots from B16 tumors 6 days after treatment.

### Example 4: Analysis of Different Classes and Subclasses of Human Allo-Antibodies with Respect to their Tumor Binding Properties and Ability to Induce DC Priming and T Cell Activation

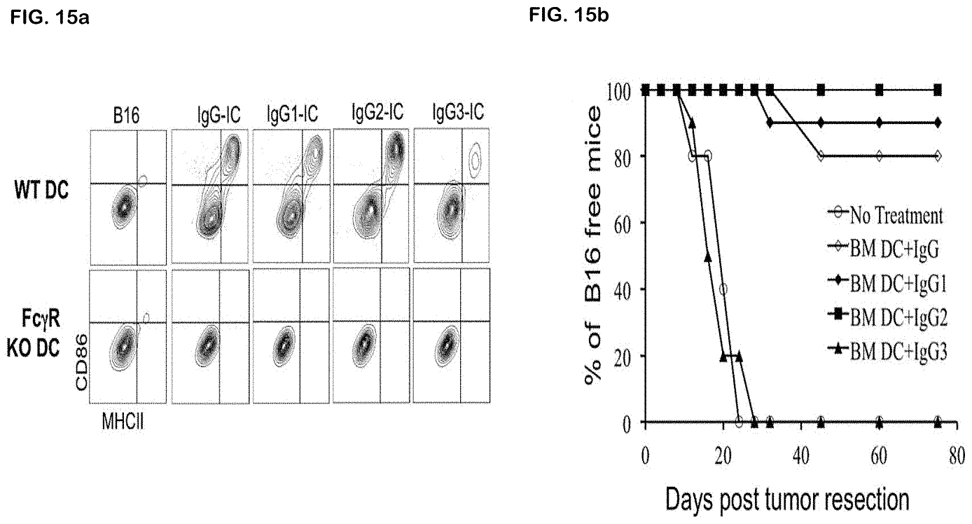

Data provided herein suggest that the anti-tumor T cell response that eradicates allogeneic tumors is mediated by the activation of APC (e.g., DC) via naturally occurring allo-antibodies, and that the effect is dependent on antibody isotype (FIG. 2G) and IgG subclass (FIG. 15). In vitro human data show that allogeneic IgG Abs bind to freshly isolated human tumor cells, that the alloIgG-tumor immune complexes (ICs) promote DC maturation, foster DC uptake of tumor-associated antigens (TAA), and facilitate autologous T cell activation by DC (FIG. 4). The differences among alloIg-IC preparations comprised of different Ig classes and subclasses, are compared in their activation of human APC (e.g., DC). To aid in the design of a clinical grade polyclonal allo-antibody preparation, the isotype (IgG, IgM, IgA, IgE) and subclass (IgG1, 2, 3, or 4) that possesses the most potent tumor binding and DC activating properties. For example, human mo-DC, TADC, and tumor cells are freshly obtained from patients with stage I and II NSCLC undergoing curative resection. The two most common NSCLC histologies, adenocarcinoma and squamous cell carcinoma, are studied. Allo-antibodies are obtained from the sera of 10 female and 10 male healthy donors, ages 20-40 years, negative for anti-HLA Abs. Access to fresh human NSCLC tissues and to healthy donor blood is readily available.

Immunofluorescence microscopy on fixed frozen human NSCLC tumor sections, and flow cytometry on FACS-purified human NSCLC tumor cells, is performed to determine whether there are differences in the degree of tumor binding between the four different subclasses of human IgG. Total human IgG, IgG subclasses (IgG1, IgG2, IgG3, IgG4), IgM, IgA, and IgE are isolated from the pooled sera of 20 healthy donors. Tumors from 8 patients undergoing resection for NSCLC are prepared for both immunofluorescence microscopy and flow cytometry. Matched “non-tumor” lung is obtained from lobectomy specimens at a site distant from the tumor and used as a control. For immunofluorescence microscopy experiments, fixed frozen human NSCLC sections are incubated with purified donor Ab fractions and then stained with fluorochrome-conjugated antibodies against human total IgG, IgG1, IgG2, IgG3, IgG4, IgM, IgA or IgE (in addition to DAPI), and the degree of allo-antibody staining is quantified using Zen software (Zeiss, Dublin, Calif.). This includes the percent of tumor area that stains positive, as well as the intensity of staining. The results are confirmed with flow cytometry. Freshly obtained human NSCLC specimens are digested for 30 min in HBSS containing DNAse I and collagenase to produce a single cell suspension, which is incubated with purified donor Ab fractions, washed, and then stained with fluorochrome-conjugated Abs against human total IgG, IgG1, IgG2, IgG3, IgG4, IgM, IgA or IgE. The median fluorescence index (MFI) of each of the different subclasses is determined on tumor cells (CD45^{neg}SSC^{high}). Autologous Abs (from the serum of the patient undergoing surgery) can serve as controls. At least two sources of commercially available intravenous immunoglobulin (IVIG) are additionally tested in these binding assays.

As Ab binding to tumor cells and APC (e.g., DC) activation can be two independent processes, subclasses of human alloIgG that possess the ability to activate human APC (e.g., DC) can be identified. Total IgG, individual IgG subclasses or IgM are incubated for 30 min with freshly isolated NSCLC human tumor cells to form alloIgG-IC. These antibody-tumor cell immune complexes are cultured overnight with autologous blood mo-DC from 8 patients undergoing resection for NSCLC in the presence of the adjuvants TNFα+CD40L The following data are obtained 1) amount or degree of DC maturation, 2) amount or degree of TAA uptake by DC, and 3) T cell stimulatory capacity of DC (as shown in FIG. 4). The ability of alloIgG-IC to activate human TADC is also examined, and identical experiments are performed in FACS-purified TADC (HLA-DR+CD3-CD19-CD56-CD14-) isolated from the tumor specimens of 5 patients. Sufficient TADC yield is obtainable with 1 cm3 tumor specimens (which yield approximately 1-5×105 TADC) and this size of tumor specimen is obtainable from most resection specimens. DC maturation is evaluated by the expression of HLA-DR (MHC-II) and the co-stimulatory molecules CD40, CD80, and CD86. DC uptake of TAA is evaluated by culturing DC with CFSE-labeled tumor cells, and using flow cytometry to detect the uptake of CFSE-labeled tumor proteins in DC. T cell activation by DC is assayed by culturing alloIgG-IC loaded DC with autologous patient blood CD4 T cells and measuring T cell proliferation by 3H-thymidine incorporation. Controls can include autologous patient Abs, and allogeneic IgA and IgE are tested to determine whether they are found to bind tumors. Additionally, the possibility that tumor-binding Abs are present in autologous serum (at lower titers) is investigated by testing IgG-ICs created by using a 10× concentration of IgG derived from the patient.

The preceding merely illustrates the principles of the invention. It will be appreciated that those skilled in the art will be able to devise various arrangements which, although not explicitly described or shown herein, embody the principles of the invention and are included within its spirit and scope. Furthermore, all examples and conditional language recited herein are principally intended to aid the reader in understanding the principles of the invention and the concepts contributed by the inventors to furthering the art, and are to be construed as being without limitation to such specifically recited examples and conditions. Moreover, all statements herein reciting principles, aspects, and embodiments of the invention as well as specific examples thereof, are intended to encompass both structural and functional equivalents thereof.

Additionally, it is intended that such equivalents include both currently known equivalents and equivalents developed in the future, i.e., any elements developed that perform the same function, regardless of structure. The scope of the present invention, therefore, is not intended to be limited to the exemplary embodiments shown and described herein. Rather, the scope and spirit of the present invention is embodied by the appended claims.

That which is claimed is:

1. A method of treating a human having cancer, the method comprising administering to the human a conjugate comprising (a) an allogeneic IgG antibody, wherein the antibody is trastuzumab, and (b) a tumor associated dendritic cell stimulatory agent, wherein the tumor associated dendritic cell stimulatory agent is a Toll-like receptor (TLR) agonist.

2. The method of claim 1, wherein the method reduces the size or number of malignant cells expressing HER2 in a tumor in the human.

3. The method of claim 1, wherein the conjugate is administered to the human intravenously.

4. The method of claim 1, wherein the conjugate is administered to the human by injection.

5. The method of claim 4, wherein the conjugate is administered to the human by subcutaneous injection.

6. The method of claim 1, wherein the conjugate is administered to the human in or near a tumor.

7. The method of claim 1, wherein the conjugate is administered to the human at a site of tumor resection.

8. The method of claim 1, further comprising administering to the human a proinflammatory cytokine.

9. The method of claim 8, wherein the proinflammatory cytokine is tumor necrosis factor alpha or interferon gamma.

10. The method of claim 1, wherein the TLR agonist is conjugated to trastuzumab with an ester linkage.

11. The method of claim 1, wherein the TLR agonist is conjugated to trastuzumab with a peptide linkage.

12. The method of claim 1, wherein the TLR agonist is a TLR-2 agonist, TLR-3 agonist, TLR-4 agonist, TLR-7 agonist, TLR-8 agonist, TLR-9 agonist, or any combination thereof.

13. The method of claim 1, wherein the TLR agonist is a TLR-3 agonist.

14. The method of claim 13, wherein the TLR-3 agonist is a poly 1:C.

15. The method of claim 1, wherein the TLR agonist is a TLR-7 agonist, TLR-8 agonist, or a combination thereof.

16. The method of claim 15, wherein the TLR agonist is an imidazoquinoline.

17. The method of claim 16, wherein the TLR agonist is imiquimod.

18. The method of claim 16, wherein the TLR agonist is resiquimod.

19. The method of claim 1, wherein the TLR agonist is a TLR-9 agonist.

20. The method of claim 19, wherein the TLR-9 agonist is a CpG ODN.


SEQUENCE LISTING (14 sequences)

SEQ ID NO: 1 - protein, 261 aa, Homo sapiens
MIETYNQTSPRSAATGLPISMKIFMYLLTVFLITQMIGSALFAVYLHRRLDKIEDERNLHEDFVFMKTIQRCNTGERSLSLLNCEEIKSQFEGFVKDIMLNKEETKKENSFEMQKGDQNPQIAAHVISEASSKTTSVLQWAEKGYYTMSNNLVTLENGKQLTVKRQGLYYIYAQVTFCSNREASSQAPFIASLCLKSPGRFERILLRAANTHSSAKPCGQQSIHLGGVFELQPGASVFVNVTDPSQVSHGTGFTSFGLLKL

SEQ ID NO: 2 - DNA, 1834 bp, Homo sapiens
actttgacagtcttctcatgctgcctctgccaccttctctgccagaagataccatttcaactttaacacagcatgatcgaaacatacaaccaaacttctccccgatctgcggccactggactgcccatcagcatgaaaatttttatgtatttacttactgtttttcttatcacccagatgattgggtcagcactttttgctgtgtatcttcatagaaggttggacaagatagaagatgaaaggaatcttcatgaagattttgtattcatgaaaacgatacagagatgcaacacaggagaaagatccttatccttactgaactgtgaggagattaaaagccagtttgaaggctttgtgaaggatataatgttaaacaaagaggagacgaagaaagaaaacagctttgaaatgcaaaaaggtgatcagaatcctcaaattgcggcacatgtcataagtgaggccagcagtaaaacaacatctgtgttacagtgggctgaaaaaggatactacaccatgagcaacaacttggtaaccctggaaaatgggaaacagctgaccgttaaaagacaaggactctattatatctatgcccaagtcaccttctgttccaatcgggaagcttcgagtcaagctccatttatagccagcctctgcctaaagtcccccggtagattcgagagaatcttactcagagctgcaaatacccacagttccgccaaaccttgcgggcaacaatccattcacttgggaggagtatttgaattgcaaccaggtgcttcggtgtttgtcaatgtgactgatccaagccaagtgagccatggcactggcttcacgtcctttggcttactcaaactctgaacagtgtcaccttgcaggctgtggtggagctgacgctgggagtcttcataatacagcacagcggttaagcccaccccctgttaactgcctatttataaccctaggatcctccttatggagaactatttattatacactccaaggcatgtagaactgtaataagtgaattacaggtcacatgaaaccaaaacgggccctgctccataagagcttatatatctgaagcagcaaccccactgatgcagacatccagagagtcctatgaaaagacaaggccattatgcacaggttgaattctgagtaaacagcagataacttgccaagttcagttttgtttctttgcgtgcagtgtctttccatggataatgcatttgatttatcagtgaagatgcagaagggaaatggggagcctcagctcacattcagttatggttgactctgggttcctatggccttgttggagggggccaggctctagaacgtctaacacagtggagaaccgaaaccccccccccccccccgccaccctctcggacagttattcattctctttcaatctctctctctccatctctctctttcagtctctctctctcaacctctttcttccaatctctctttctcaatctctctgtttccctttgtcagtctcttccctcccccagtctctcttctcaatccccctttctaacacacacacacacacacacacacacacacacacacacacacacacacacacagagtcaggccgttgctagtcagttctcttctttccaccctgtccctatctctaccactatagatgagggtgaggagtagggagtgcagccctgagcctgcccactcctcattacgaaatgactgtatttaaaggaaatctattgtatctacctgcagtctccattgtttccagagtgaacttgtaattatcttgttatttattttttgaataataaagacctcttaacattaa

SEQ ID NO: 3 - protein, 149 aa, Artificial sequence
  Synthetic sequence
MQRGDEDPQIAAHVVSEANSNAASVLQWAKKGYYTMKSNLVMLENGKQLTVKREGLYYVYTQVTFCSNREPSSQRPFIVGLWLKPSSGSERILLKAANTHSSSQLCEQQSVHLGGVFELQAGASVFVNVTEASQVIHRVGFSSFGLLKL

SEQ ID NO: 4 - protein, 149 aa, Artificial sequence
  Synthetic sequence
MQKGDQNPQIAAHVISEASSKTTSVLQWAEKGYYTMSNNLVTLENGKQLTVKRQGLYYIYAQVTFCSNREASSQAPFIASLWLKSPGRFERILLRAANTHSSAKPCGQQSIHLGGVFELQPGASVFVNVTDPSQVSHGTGFTSFGLLKL

SEQ ID NO: 5 - protein, 233 aa, Homo sapiens
MSTESMIRDVELAEEALPKKTGGPQGSRRCLFLSLFSFLIVAGATTLFCLLHFGVIGPQREEFPRDLSLISPLAQAVRSSSRTPSDKPVAHVVANPQAEGQLQWLNRRANALLANGVELRDNQLVVPSEGLYLIYSQVLFKGQGCPSTHVLLTHTISRIAVSYQTKVNLLSAIKSPCQRETPEGAEAKPWYEPIYLGGVFQLEKGDRLSAEINRPDYLDFAESGQVYFGIIAL

SEQ ID NO: 6 - DNA, 1686 bp, Homo sapiens
cagacgctccctcagcaaggacagcagaggaccagctaagagggagagaagcaactacagaccccccctgaaaacaaccctcagacgccacatcccctgacaagctgccaggcaggttctcttcctctcacatactgacccacggctccaccctctctcccctggaaaggacaccatgagcactgaaagcatgatccgggacgtggagctggccgaggaggcgctccccaagaagacaggggggccccagggctccaggcggtgcttgttcctcagcctcttctccttcctgatcgtggcaggcgccaccacgctcttctgcctgctgcactttggagtgatcggcccccagagggaagagttccccagggacctctctctaatcagccctctggcccaggcagtcagatcatcttctcgaaccccgagtgacaagcctgtagcccatgttgtagcaaaccctcaagctgaggggcagctccagtggctgaaccgccgggccaatgccctcctggccaatggcgtggagctgagagataaccagctggtggtgccatcagagggcctgtacctcatctactcccaggtcctcttcaagggccaaggctgcccctccacccatgtgctcctcacccacaccatcagccgcatcgccgtctcctaccagaccaaggtcaacctcctctctgccatcaagagcccctgccagagggagaccccagagggggctgaggccaagccctggtatgagcccatctatctgggaggggtcttccagctggagaagggtgaccgactcagcgctgagatcaatcggcccgactatctcgactttgccgagtctgggcaggtctactttgggatcattgccctgtgaggaggacgaacatccaaccttcccaaacgcctcccctgccccaatccctttattaccccctccttcagacaccctcaacctcttctggctcaaaaagagaattgggggcttagggtcggaacccaagcttagaactttaagcaacaagaccaccacttcgaaacctgggattcaggaatgtgtggcctgcacagtgaagtgctggcaaccactaagaattcaaactggggcctccagaactcactggggcctacagctttgatccctgacatctggaatctggagaccagggagcctttggttctggccagaatgctgcaggacttgagaagacctcacctagaaattgacacaagtggaccttaggccttcctctctccagatgtttccagacttccttgagacacggagcccagccctccccatggagccagctccctctatttatgtttgcacttgtgattatttattatttatttattatttatttatttacagatgaatgtatttatttgggagaccggggtatcctgggggacccaatgtaggagctgccttggctcagacatgttttccgtgaaaacggagctgaacaataggctgttcccatgtagccccctggcctctgtgccttcttttgattatgttttttaaaatatttatctgattaagttgtctaaacaatgctgatttggtgaccaactgtcactcattgctgagcctctgctccccaggggagttgtgtctgtaatcgccctactattcagtggcgagaaataaagtttgcttagaaaagaaaaaaaaaaaaa

SEQ ID NO: 7 - protein, 271 aa, Homo sapiens
MAKVPDMFEDLKNCYSENEEDSSSIDHLSLNQKSFYHVSYGPLHEGCMDQSVSLSISETSKTSKLTFKESMVVVATNGKVLKKRRLSLSQSITDDDLEAIANDSEEEIIKPRSAPFSFLSNVKYNFMRIIKYEFILNDALNQSIIRANDQYLTAAALHNLDEAVKFDMGAYKSSKDDAKITVILRISKTQLYVTAQDEDQPVLLKEMPEIPKTITGSETNLLFFWETHGTKNYFTSVAHPNLFIATKQDYWVCLAGGPPSITDFQILENQA

SEQ ID NO: 8 - DNA, 2943 bp, Homo sapiens
accaggcaacaccattgaaggctcatatgtaaaaatccatgccttcctttctcccaatctccattcccaaacttagccactggcttctggctgaggccttacgcatacctcccggggcttgcacacaccttcttctacagaagacacaccttgggcatatcctacagaagaccaggcttctctctggtccttggtagagggctactttactgtaacagggccagggtggagagttctctcctgaagctccatcccctctataggaaatgtgttgacaatattcagaagagtaagaggatcaagacttctttgtgctcaaataccactgttctcttctctaccctgccctaaccaggagcttgtcaccccaaactctgaggtgatttatgccttaatcaagcaaacttccctcttcagaaaagatggctcattttccctcaaaagttgccaggagctgccaagtattctgccaattcaccctggagcacaatcaacaaattcagccagaacacaactacagctactattagaactattattattaataaattcctctccaaatctagccccttgacttcggatttcacgatttctcccttcctcctagaaacttgataagtttcccgcgcttccctttttctaagactacatgtttgtcatcttataaagcaaaggggtgaataaatgaaccaaatcaataacttctggaatatctgcaaacaacaataatatcagctatgccatctttcactattttagccagtatcgagttgaatgaacatagaaaaatacaaaactgaattcttccctgtaaattccccgttttgacgacgcacttgtagccacgtagccacgcctacttaagacaattacaaaaggcgaagaagactgactcaggcttaagctgccagccagagagggagtcatttcattggcgtttgagtcagcaaagaagtcaagatggccaaagttccagacatgtttgaagacctgaagaactgttacagtgaaaatgaagaagacagttcctccattgatcatctgtctctgaatcagaaatccttctatcatgtaagctatggcccactccatgaaggctgcatggatcaatctgtgtctctgagtatctctgaaacctctaaaacatccaagcttaccttcaaggagagcatggtggtagtagcaaccaacgggaaggttctgaagaagagacggttgagtttaagccaatccatcactgatgatgacctggaggccatcgccaatgactcagaggaagaaatcatcaagcctaggtcagcaccttttagcttcctgagcaatgtgaaatacaactttatgaggatcatcaaatacgaattcatcctgaatgacgccctcaatcaaagtataattcgagccaatgatcagtacctcacggctgctgcattacataatctggatgaagcagtgaaatttgacatgggtgcttataagtcatcaaaggatgatgctaaaattaccgtgattctaagaatctcaaaaactcaattgtatgtgactgcccaagatgaagaccaaccagtgctgctgaaggagatgcctgagatacccaaaaccatcacaggtagtgagaccaacctcctcttcttctgggaaactcacggcactaagaactatttcacatcagttgcccatccaaacttgtttattgccacaaagcaagactactgggtgtgcttggcaggggggccaccctctatcactgactttcagatactggaaaaccaggcgtaggtctggagtctcacttgtctcacttgtgcagtgttgacagttcatatgtaccatgtacatgaagaagctaaatcctttactgttagtcatttgctgagcatgtactgagccttgtaattctaaatgaatgtttacactctttgtaagagtggaaccaacactaacatataatgttgttatttaaagaacaccctatattttgcatagtaccaatcattttaattattattcttcataacaattttaggaggaccagagctactgactatggctaccaaaaagactctacccatattacagatgggcaaattaaggcataagaaaactaagaaatatgcacaatagcagttgaaacaagaagccacagacctaggatttcatgatttcatttcaactgtttgccttctacttttaagttgctgatgaactcttaatcaaatagcataagtttctgggacctcagttttatcattttcaaaatggagggaataatacctaagccttcctgccgcaacagttttttatgctaatcagggaggtcattttggtaaaatacttcttgaagccgagcctcaagatgaaggcaaagcacgaaatgttattttttaattattatttatatatgtatttataaatatatttaagataattataatatactatatttatgggaaccccttcatcctctgagtgtgaccaggcatcctccacaatagcagacagtgttttctgggataagtaagtttgatttcattaatacagggcattttggtccaagttgtgcttatcccatagccaggaaactctgcattctagtacttgggagacctgtaatcatataataaatgtacattaattaccttgagccagtaattggtccgatctttgactcttttgccattaaacttacctgggcattcttgtttcaattccacctgcaatcaagtcctacaagctaaaattagatgaactcaactttgacaaccatgagaccactgttatcaaaactttcttttctggaatgtaatcaatgtttcttctaggttctaaaaattgtgatcagaccataatgttacattattatcaacaatagtgattgatagagtgttatcagtcataactaaataaagcttgcaacaaaattctctgacaaaaaaaaaaaaaaaa

SEQ ID NO: 9 - protein, 269 aa, Homo sapiens
MAEVPELASEMMAYYSGNEDDLFFEADGPKQMKCSFQDLDLCPLDGGIQLRISDHHYSKGFRQAASVVVAMDKLRKMLVPCPQTFQENDLSTFFPFIFEEEPIFFDTWDNEAYVHDAPVRSLNCTLRDSQQKSLVMSGPYELKALHLQGQDMEQQVVFSMSFVQGEESNDKIPVALGLKEKNLYLSCVLKDDKPTLQLESVDPKNYPKKKMEKRFVFNKIEINNKLEFESAQFPNWYISTSQAENMPVFLGGTKGGQDITDFTMQFVSS

SEQ ID NO: 10 - DNA, 1498 bp, Homo sapiens
accaaacctcttcgaggcacaaggcacaacaggctgctctgggattctcttcagccaatcttcattgctcaagtgtctgaagcagccatggcagaagtacctgagctcgccagtgaaatgatggcttattacagtggcaatgaggatgacttgttctttgaagctgatggccctaaacagatgaagtgctccttccaggacctggacctctgccctctggatggcggcatccagctacgaatctccgaccaccactacagcaagggcttcaggcaggccgcgtcagttgttgtggccatggacaagctgaggaagatgctggttccctgcccacagaccttccaggagaatgacctgagcaccttctttcccttcatctttgaagaagaacctatcttcttcgacacatgggataacgaggcttatgtgcacgatgcacctgtacgatcactgaactgcacgctccgggactcacagcaaaaaagcttggtgatgtctggtccatatgaactgaaagctctccacctccagggacaggatatggagcaacaagtggtgttctccatgtcctttgtacaaggagaagaaagtaatgacaaaatacctgtggccttgggcctcaaggaaaagaatctgtacctgtcctgcgtgttgaaagatgataagcccactctacagctggagagtgtagatcccaaaaattacccaaagaagaagatggaaaagcgatttgtcttcaacaagatagaaatcaataacaagctggaatttgagtctgcccagttccccaactggtacatcagcacctctcaagcagaaaacatgcccgtcttcctgggagggaccaaaggcggccaggatataactgacttcaccatgcaatttgtgtcttcctaaagagagctgtacccagagagtcctgtgctgaatgtggactcaatccctagggctggcagaaagggaacagaaaggtttttgagtacggctatagcctggactttcctgttgtctacaccaatgcccaactgcctgccttagggtagtgctaagaggatctcctgtccatcagccaggacagtcagctctctcctttcagggccaatccccagcccttttgttgagccaggcctctctcacctctcctactcacttaaagcccgcctgacagaaaccacggccacatttggttctaagaaaccctctgtcattcgctcccacattctgatgagcaaccgcttccctatttatttatttatttgtttgtttgttttattcattggtctaatttattcaaagggggcaagaagtagcagtgtctgtaaaagagcctagtttttaatagctatggaatcaattcaatttggactggtgtgctctctttaaatcaagtcctttaattaagactgaaaatatataagctcagattatttaaatgggaatatttataaatgagcaaatatcatactgttcaatggttctgaaataaacttcactgaag

SEQ ID NO: 11 - protein, 215 aa, Homo sapiens
MCTEGAFPHRSACSLPLTHVHTHIHVCVPVLWGSVPRGMKLQCVSLWLLGTILILCSVDNHGLRRCLISTDMHHIEESFQEIKRAIQAKDTFPNVTILSTLETLQIIKPLDVCCVTKNLLAFYVDRVFKDHQEPNPKILRKISSIANSFLYMQKTLRQCQEQRQCHCRQEATNATRVIHDNYDQLEVHAAAIKSLGELDVFLAWINKNHEVMFSA

SEQ ID NO: 12 - DNA, 1048 bp, Homo sapiens
tgcacacactgacaggagtccaagaatgtgcactgagggagcgtttccgcacagatctgcgtgttccttaccactcacacatgtgcacacacatatccatgtgtgtgtgccagtgctttggggctctgttccacggggcatgaagttacagtgtgtttccctttggctcctgggtacaatactgatattgtgctcagtagacaaccacggtctcaggagatgtctgatttccacagacatgcaccatatagaagagagtttccaagaaatcaaaagagccatccaagctaaggacaccttcccaaatgtcactatcctgtccacattggagactctgcagatcattaagcccttagatgtgtgctgcgtgaccaagaacctcctggcgttctacgtggacagggtgttcaaggatcatcaggagccaaaccccaaaatcttgagaaaaatcagcagcattgccaactctttcctctacatgcagaaaactctgcggcaatgtcaggaacagaggcagtgtcactgcaggcaggaagccaccaatgccaccagagtcatccatgacaactatgatcagctggaggtccacgctgctgccattaaatccctgggagagctcgacgtctttctagcctggattaataagaatcatgaagtaatgttctcagcttgatgacaaggaacctgtatagtgatccagggatgaacaccccctgtgcggtttactgtgggagacagcccaccttgaaggggaaggagatggggaaggccccttgcagctgaaagtcccactggctggcctcaggctgtcttattccgcttgaaaatagccaaaaagtctactgtggtatttgtaataaactctatctgctgaaagggcctgcaggccatcctgggagtaaagggctgccttcccatctaatttattgtaaagtcatatagtccatgtctgtgatgtgagccaagtgatatcctgtagtacacattgtactgagtggtttttctgaataaattccatattttacctatgaaaaaaaaaaaaaaaaaa

SEQ ID NO: 13 - protein, 177 aa, Homo sapiens
MKLQCVSLWLLGTILILCSVDNHGLRRCLISTDMHHIEESFQEIKRAIQAKDTFPNVTILSTLETLQIIKPLDVCCVTKNLLAFYVDRVFKDHQEPNPKILRKISSIANSFLYMQKTLRQCQEQRQCHCRQEATNATRVIHDNYDQLEVHAAAIKSLGELDVFLAWINKNHEVMFSA

SEQ ID NO: 14 - DNA, 1848 bp, Homo sapiens
gctggagtgcaatggtgaaattatagcagactgcagtcttcaactcctgacctcaagcaattgtcctgcctcctcaacttcctgactacaggtgtgcatgaggactacaggcaggcatgtgccaacacatgcagctttttttttttttttttttcagagatgtggtctcgctttgttgcctacactggtctcaaactcttggcctcaagggatcctcccacctcggcttcccaaagtgcagagattacagtctcattttctctctctctgcattaatcaagaatgagagaaccctccaggggacaagatgaaggggaaatagatgatgtgcaaagaaatccttgctttatgaggggaaaaagtgttcctcatgaagttcaacaaaatgatgcaggtaaagcagttagctagcacctggcacatggcagacactcatagctgcctaaggcattggagaactggatcgtgctgcagccagaggcacctgcagagcctcatgggctggctgctgcagggtgtggctgattgagagtgcttttgtgagttggcctgcagggtacacttggtaacgtgccacagctctcaggaaagtgacctaagttggatttttctgcatggacatagaattgcaaaaaattctcatttgcatggagatggggagtttatttttcctagaagctgcatgtcaagacccagaagaaagaggcatttcataataatgattaatcagctatatcttaaagaagaaagaaaacaattaaggaaatacaatactaagaaaacaaggggaaaaaacaatctccccaaggtggatccacccagcaaaccttgacagcatttcctcttatccacctgaataaaaatgaccagccctttccaaatggcagagagcactgagaggagacacaaggagcagcccgcaagcaccaagtgagaggcatgaagttacagtgtgtttccctttggctcctgggtacaatactgatattgtgctcagtagacaaccacggtctcaggagatgtctgatttccacagacatgcaccatatagaagagagtttccaagaaatcaaaagagccatccaagctaaggacaccttcccaaatgtcactatcctgtccacattggagactctgcagatcattaagcccttagatgtgtgctgcgtgaccaagaacctcctggcgttctacgtggacagggtgttcaaggatcatcaggagccaaaccccaaaatcttgagaaaaatcagcagcattgccaactctttcctctacatgcagaaaactctgcggcaatgtcaggaacagaggcagtgtcactgcaggcaggaagccaccaatgccaccagagtcatccatgacaactatgatcagctggaggtccacgctgctgccattaaatccctgggagagctcgacgtctttctagcctggattaataagaatcatgaagtaatgttctcagcttgatgacaaggaacctgtatagtgatccagggatgaacaccccctgtgcggtttactgtgggagacagcccaccttgaaggggaaggagatggggaaggccccttgcagctgaaagtcccactggctggcctcaggctgtcttattccgcttgaaaatagccaaaaagtctactgtggtatttgtaataaactctatctgctgaaagggcctgcaggccatcctgggagtaaagggctgccttcccatctaatttattgtaaagtcatatagtccatgtctgtgatgtgagccaagtgatatcctgtagtacacattgtactgagtggtttttctgaataaattccatattttacctatgaaaaaaaaaaaaaaaaaa